(19) United States
(12) Patent Application Publication
(10) Pub. No.: US 2025/0154403 A1
(43) Pub. Date: May 15, 2025

(54) LUMINESCENT NANOPARTICLE AND LUMINESCENT LABELING MATERIAL FOR PATHOLOGICAL DIAGNOSIS USE

(71) Applicant: Konica Minolta, Inc., Tokyo (JP)

(72) Inventors: Yasuo MIYATA, Yokohama-shi, Kanagawa (JP); Kazuma ODA, Hachioji-shi, Tokyo (JP); Shigeru KOJIMA, Inagi-shi, Tokyo (JP); Shumpei HITOSUGI, Hachioji-shi, Tokyo (JP)

(21) Appl. No.: 18/682,815

(22) PCT Filed: Mar. 17, 2022
(86) PCT No.: PCT/JP2022/012359

§ 371 Date: Feb. 9, 2024

(30) Foreign Application Priority Data
Aug. 30, 2021 (JP) 2021-139818

(51) Int. Cl. C09K 11/06; C07D 471/06; G01N 33/531

(52) U.S. Cl. CPC C09K 11/06; C07D 471/06; G01N 33/531

(57) ABSTRACT

A luminescent nanoparticle contains a first luminescent compound and a second luminescent compound. The first luminescent compound is excited by light irradiation and has a function of transferring energy due to the excitation to the second luminescent compound. The second luminescent compound has a function of receiving the energy due to the excitation to emit light. A content of the first luminescent compound to a total amount of the luminescent nanoparticle is in the range of 4 to 90% by mass.

### TECHNICAL FIELD

The present invention relates to a luminescent nanoparticle and a luminescent labeling material for pathological diagnosis use, and more particularly, to a luminescent nanoparticle and a luminescent labeling material for pathological diagnosis use that realize a high-brightness particulate technique for bioimaging and enable high-sensitivity imaging.

### BACKGROUND ART

In “bioimaging” using a luminescent compound, it is a major challenge to separate luminesce of a luminescent compound from cellular autofluorescence in order to achieve high-sensitivity imaging available for positions and quantification of protein due to bright spots of nanoparticles in an imaging image. Note that “bioimaging” refers to observation of structures of targets such as cells or protein and states of positions and movements of the targets in a living body by using luminescence from a luminescent (e.g., fluorescent) probe such as a luminescent fine particle after specific adsorption of the luminescent (e.g., fluorescent) probe to the targets.

As means for avoiding the adverse effects of autofluorescence on the bioimaging, there exists means utilizing phenomena such as near-infrared luminescence, long Stokes shift luminescence, and delayed luminescence.

In long Stokes shift luminescence and delayed luminescence, a luminescent compound consisting of a structure including a donor-site and an acceptor-site is used to generate luminescence by intramolecular electron transfer due to excitation of either site. Therefore, the molecular designs of a luminescent compound are limited, and the control of the excitation wavelength and the luminescent wavelength is not free. In both cases, it is difficult in view of molecular design to extend the π-conjugated system of the donor site and the acceptor site in order to improve molar absorption coefficient of a luminescent compound for increasing brightness and to increase the wavelength of the absorbed wavelength, because it causes a decrease in luminescence due to promotion of electron transfer state or a decrease in the excited triplet level in the ground-state.

Therefore, in long Stokes shift luminescence material and delayed luminescence material, it is difficult to improve molar absorption coefficient and to design the excitation wavelength in a wavelength range (450 nm or more) in which the cells are not deteriorated. Moreover, because delayed luminescence cannot be detected on a general-purpose device, its use in hospitals and clinics is impractical.

On the other hand, near-infrared luminescence is biopermeable and can avoid cellular autofluorescence, so it is widely used in bioimaging. However, the luminesce in the near-infrared region is fundamentally problematic in that quantum yield is lower due to the energy-gap law. In addition, in common water bioimaging, solvent of water to a luminescent compound promotes the transition from an excited state to a lower-energy-level charge-separated state, causing a decrease in quantum yield.

In the process using luminescent nanoparticle which can relax solvation effect of water, there is a problem of a decrease in luminescence property due to a luminescent compound caused by aggregation quenching caused by packing a luminescent compound in the nanoparticle, and the effect of aggregation quenching is very large in quantum yield lower near-infrared luminescence due to the energy-gap law. On the other hand, when s structure of a luminescent compound is made rigid in order to maintain quantum yield, the maximum absorption wavelength and the maximum luminescence wavelength are close to each other, so that the excitation light becomes stray light and becomes noisy on imaging, which may hinder high-sensitivity imaging.

Patent Document 1 and Patent Document 2 disclose a technique in which two kinds of luminescent dyes (a luminescent compound) are introduced into a nanoparticle, light excitation is performed using the dye of the first component as an energy donor, and the dye of the second component emits light as an energy acceptor via energy transfer. In these conventional techniques, stray light of cellular autofluorescence and excitation light can be avoided, but since a luminescent compound content in the particles is designed to be very small in order to maintain quantum yield of a luminescent compound, the brightness effect due to low absorbance and a luminescent compound degradation becomes sensitive, and the particle brightness (=absorbance×quantum yield) is problematic in high-sensitivity imaging applications.

### CITATION LIST

### Patent Document

Patent Document 1: Japanese Patent No. 3773949
Patent Document 2: Japanese Patent No. 5306714

### SUMMARY OF INVENTION

### Technical Problem

The present invention has been made in view of the above-described problems and circumstances, and an object according to the present invention is to provide a luminescent nanoparticle and a luminescent labeling material for pathological diagnosis use that realize a high-brightness particulate technique for bioimaging and enable high-sensitivity imaging.

### Solution to Problem

In order to solve the above problems, the present inventors have studied the causes and the like of the above problems, and found, in order to reach the present invention, that a high-brightness particulate technique for bioimaging is realized and high-sensitivity imaging becomes enable, by causing a luminescent nanoparticle to include a first luminescent compound that is excited by light irradiation and has a function of transferring energy due to the excitation to a second luminescent compound, and the second luminescent compound that has a function of receiving the energy due to the excitation to emit light, and by causing a content of the first luminescent compound to a total amount of the luminescent nanoparticle to be in the range of 4 to 90% by mass. That is, the above-described problem according to the present invention is solved by the following means.

[1] A luminescent nanoparticle comprising a first luminescent compound and a second luminescent compound, wherein
the first luminescent compound is excited by light irradiation and has a function of transferring energy due to the excitation to the second luminescent compound,
the second luminescent compound has a function of receiving the energy due to the excitation to emit light, and
a content of the first luminescent compound to a total amount of the luminescent nanoparticle is in the range of 4 to 90% by mass.

[2] The luminescent nanoparticle according to [1], wherein the first luminescent compound has a structure represented by the following general formula (1), general formula (2), or general formula (3).

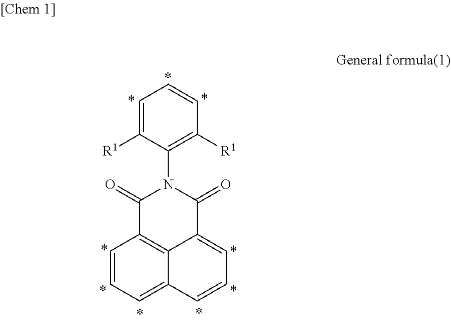

In the general formula (1), each of the plurality of R¹ independently represents a hydrogen atom or a substituent, and at least one of them represents a monovalent organic group having 3 to 30 carbon atoms. The benzene ring or the naphthalene ring may further have a substituent, and * represents a position of the substituent which may be included in the benzene ring or the naphthalene ring.

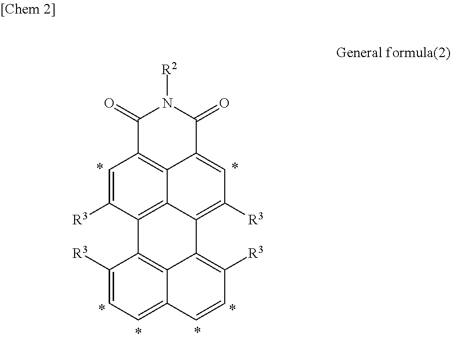

In the general formula (2), R² represents a substituted or unsubstituted alkyl group, an aryl group or a heteroaryl group. Each of the plurality of R³ independently represents a hydrogen-atom or a group having a structure represented by the following general formula (F1), and at least one of them represents a group having a structure represented by the following general formula (F1). The naphthalene ring may further have a substituent, and * represents a position of the substituent that may be included in the naphthalene ring.

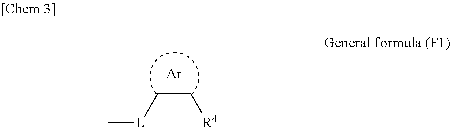

In the general formula (F1), Ar represents an aryl ring or a heteroaryl ring. R⁴ represents a substituent. When two or more groups represented by the general formula (F1) are included, the two R⁴ may be linked to each other. L represents a single bond, an oxygen-atom, a sulfur-atom or —NR′—. R′ represents a hydrogen atom, an alkyl group, an aryl group or a heteroaryl group.

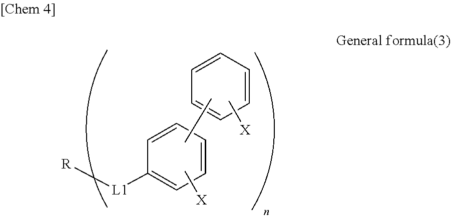

In the general formula (3), R represents a luminescent compound skeleton. Each X independently represents an ionic substituent. L1 represents a single bond, an oxygen-atom, a sulfur-atom, a selenium-atom or a NH group. n represents an integer of 1 or more.

[3] The luminescent nanoparticle according to [1], wherein the first luminescent compound comprises a compound having a structure represented by the following general formula (1c), the general formula (1d), or the general formula (1e):

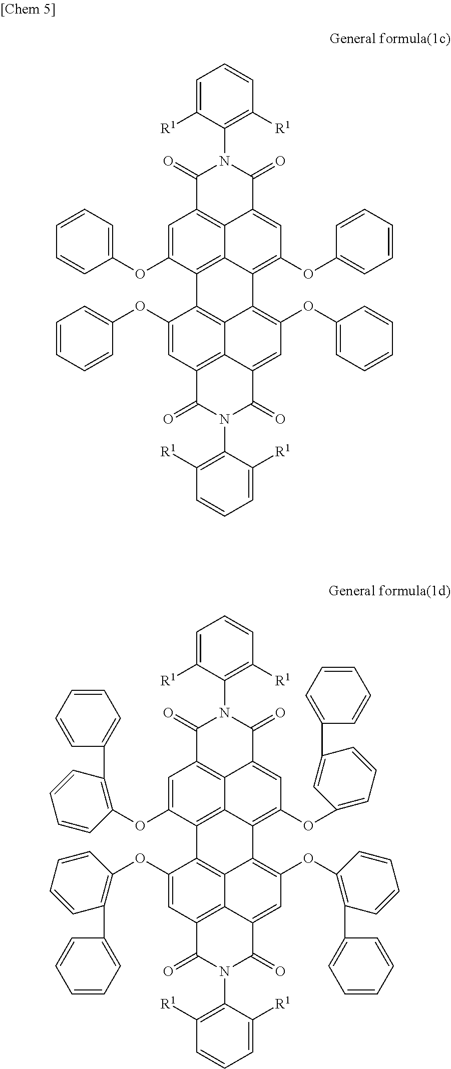

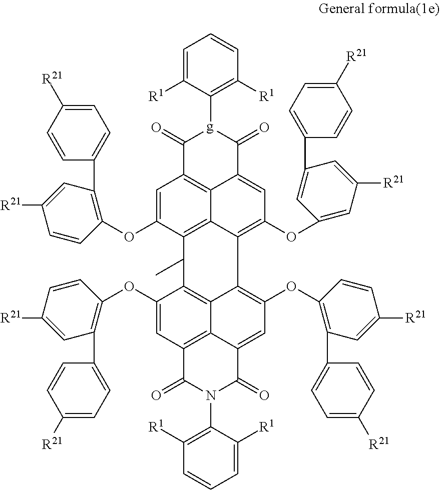

In general formulae (1c) to (1e), a plurality of R¹ each independently represents a hydrogen atom or a substituent, and at least one of them represents a monovalent organic group having 3 to 30 carbon atoms. In the general formula (1e), R²¹ independently represents a hydrogen-atom or an ionic substituent, and at least one of them represents an ionic substituent.

[4] The luminescent nanoparticle according to any one of [1] to [3], wherein a molar ratio of the second luminescent compound to the first luminescent compound is in the range of 1:2 to 1:200.

[5] The luminescent nanoparticle according to any one of [1] to [4], wherein the second luminescent compound is a xanthene dye.

[6] The luminescent nanoparticle according to any one of [1] to [5], further comprising a binder.

[7] The luminescent nanoparticle according to any one of [1] to [6], having a hydrophilic group on a surface thereof.

[8] A luminescent labeling material for pathological diagnosis use in which the luminescent nanoparticle according to any one of [1] to [7] is used.

### Advantageous Effects of Invention

The present invention provides a luminescent nanoparticle and a luminescent labeling material for pathological diagnosis use capable of realizing a high-brightness particulate technique for bioimaging and enabling high-sensitivity imaging. In particular, this technique is useful for biopermeable high-sensitivity imaging in the near-infrared range where the luminesce quantum yield is lower.

The realization mechanism of or the action mechanism of the effects according to the present invention has not been clarified, but it is presumed as follows.

In the luminescent nanoparticle according to the present invention, at least two types of luminescent compounds are contained, and the functions thereof are separated. That is, in order to avoid cellular autofluorescence due to large separations of the excitation wavelength and the luminescence wavelength, the control of the wavelength designs of the excitation and luminescence, which is a problem that has occurred in conventional single-molecule luminescence imaging techniques, has been enabled by means of using nanoparticles that contain two or more types of luminescent compounds.

Specifically, in the luminescent nanoparticle according to the present invention, the functions are separated into a first luminescent compound excited by light irradiation and a second luminescent compound that receives and emits the excited energy of the first luminescent compound. More specifically, a luminescent compound a content of which to a total amount of the luminescent nanoparticle is high such as in the range of 4 to 90% by mass and in which aggregation quenching is suppressed was used as the first luminescent compound.

This allows the first luminescent compound to be contained in the luminescent nanoparticle at high concentrations, maximizing absorbance and energy-transfer efficiency. The second luminescent compound can emit light with high brightness by receiving the maximized energy even if a content of the second luminescent compound is low, for example.

As will be described later, typically, in a relation between the maximum luminescence wavelength of the first luminescent compound and the maximum absorption wavelength of the second luminescent compound, the maximum absorption wavelength of the second luminescent compound is located at a longer wavelength than the maximum luminescence wavelength of the first luminescent compound. In the present invention, it is considered that the nanoparticle technology that satisfies high brightness and long Stokes shift luminesce is realized by such a mechanism.

In particular, when the second luminescent compound emits near-infrared light, a content of the second luminescent compound can be suppressed to be low, and thereby aggregation quenching is suppressed, so that the effect that the quantum yield is maintained is high.

In this way, it is believed that the present invention can provide a luminescent nanoparticle that realizes a high-brightness particulate technique for bioimaging and enables high-sensitivity imaging.

### DESCRIPTION OF EMBODIMENTS

The luminescent nanoparticle according to the present invention is characterized in a luminescent nanoparticle including a first luminescent compound and a second luminescent compound, wherein the first luminescent compound is excited by light irradiation and has a function of transferring energy due to the excitation to the second luminescent compound, the second luminescent compound has a function of receiving the energy due to the excitation to emit light, and a content of the first luminescent compound to a total amount of the luminescent nanoparticle is in the range of 4 to 90% by mass. This feature is the same as or corresponding to the technical feature of embodiment described below.

In the present invention, it is preferable that the first luminescent compound is a luminescent compound which has the maximal value of energy due to the excitation (hereinafter, also referred to as “excitation energy”) within a range of 4 to 90% by mass as a content of the first luminescent compound with respect to the total amount of the luminescent nanoparticle. In other words, it is preferable that the first luminescent compound is a compound in which within a range of 4 to 90% by mass as a content of the first luminescent compound with respect to the total amount of the luminescent nanoparticle, a relatively large number of molecules of the first luminescent compound absorb excitation light and efficiency for transferring the obtained excitation energy to the second luminescent compound becomes the maximum.

In the present invention, when a content of the second luminescent compound is set to a constant amount, the luminescence intensity of the second luminescent compound preferably has the maximum within a range of 4 to 90% by mass of a content of the first luminescent compound to the total amount of the luminescent nanoparticle.

As an embodiment according to the present invention, from the viewpoint of realization of the effectiveness according to the present invention, it is preferable that the first luminescent compound has a structure represented by general formula (1), general formula (2), or general formula (3). Further, it is preferable that the first luminescent compound includes a compound having a structure represented by the general formula (1c), the general formula (1d), or the general formula (1e). These compounds are preferred, for example, from the viewpoint of having an appropriate absorbance and the maximum absorption wavelength for excitation light and being efficient in transferring energy from the excited molecules, within a range of 4 to 90% by mass of a content of the first luminescent compound to the total amount of the luminescent nanoparticle.

In an embodiment according to the present invention, from the viewpoint of the effectiveness according to the present invention, it is preferable that a molar ratio of the second luminescent compound to the first luminescent compound is preferably in the range of 1:2 to 1:200.

In an embodiment according to the present invention, from the viewpoint of the effectiveness according to the present invention and biopermeablity of light in the near-infrared region, it is preferable that the second luminescent compound is a luminescent compound that emits near-infrared light. For the same reason, it is preferable that the second luminescent compound is a xanthene dye.

In an embodiment according to the present invention, from the viewpoint of the effectiveness according to the present invention, it is preferable that the luminescent nanoparticle further includes a binder.

In an embodiment according to the present invention, from the viewpoint of the effectiveness according to the present invention, it is preferable that the luminescent nanoparticle has a hydrophilic group on a surface thereof.

A luminescent labeling material for pathological diagnosis use according to the present invention is characterized in that the luminescent nanoparticle according to the present invention is used.

Hereinafter, the present invention, its constituent elements, and embodiments and aspects for carrying out the present invention will be described in detail. In the present application, “-” is used to include numerical values described before and after the numerical values as the lower limit value and the upper limit value.

### [Luminescent Nanoparticle]

The luminescent nanoparticle according to the present invention is characterized in a luminescent nanoparticle including a first luminescent compound and a second luminescent compound, wherein the first luminescent compound is excited by light irradiation and has a function of transferring energy due to the excitation to the second luminescent compound, the second luminescent compound has a function of receiving the energy due to the excitation to emit light, and a content of the first luminescent compound to a total amount of the luminescent nanoparticle is in the range of 4 to 90% by mass.

Here, the first luminescent compound is a compound which, when used alone, can absorb a predetermined excitation light corresponding to a purpose to be excited and emit light. As to the relation to the second luminescent compound, it is capable of transferring the excited energy to the second luminescent compound. In the luminescent nanoparticle according to the present invention, the first luminescent compound is excited to transfer the excited energy to the second luminescent compound and the second luminescent compound emits light, so that the first luminescent compound does not emit light.

The relation between the maximum luminescence wavelength of the first luminescent compound and the maximum absorption wavelength of the second luminescent compound will be described below. In the following explanation, the maximum luminescence wavelength of the first luminescent compound and the maximum absorption wavelength of the second luminescent compound are the maximum luminescence wavelength and the maximum absorption wavelength measured independently for the first luminescent compound and the second luminescent compound, respectively.

As described above, the relation between the maximum luminescence wavelength of the first luminescent compound and the maximum absorption wavelength of the second luminescent compound is preferably λₑₘ₁<λ_{ab2}, where λₑₘ₁ is the maximum luminescence wavelength of the first luminescent compound and λ_{ab2} is the maximum absorption wavelength of the second luminescent compound.

The energy transfer between the first luminescent compound and the second luminescent compound is typically a Forster-type energy transfer in which the luminescent spectrum of the first luminescent compound and the absorption spectrum of the second luminescent compound overlap. Here, a dexter-type energy transfer may occur at the same time.

In order to make the above-described Ferster-type energy transfer efficient, the difference between λ_{ab2} and λₑₘ₁ represented by λ_{ab2}−λₑₘ₁ is preferably equal to or less than 70 nm, and more preferably equal to or less than 50 nm.

The maximum luminescence wavelength of the second luminescent compound is shown in λₑₘ₂. It is preferable that λₑₘ₂ is in the near-infrared range from the viewpoint of the remarkable effectiveness according to the present invention and biopermeablity of light in the near-infrared region. In the present specification, the near-infrared region refers to a region of 650-1800 nm. More preferably, λₑₘ₂ is in 650-1000 nm.

As used herein, “luminescent nanoparticle” refers to a particle including a luminescent compound and having, for example, a average particle size ranging from 1 to 1000 nm. The average particle size is preferably in the range of 30-500 nm, more preferably in the range of 50-200 nm.

The average particle size of the luminescent nanoparticle can be determined by methods known in the art. Specifically, an electron micrograph can be taken using a scanning electron microscope (SEM) at an appropriate magnification, and the cross-sectional area of the luminescent nanoparticle can be measured, and the average particle size is measured as a diameter (area circle equivalent diameter) when the cross-sectional area is taken as the area of the corresponding circle.

The average (average particle size) and coefficient of variation of the particle size of the population of the luminescent nanoparticle are calculated as the arithmetic average of the particle size after measuring the particle size as described above for a sufficient number (e.g., 1,000) of the luminescent nanoparticles, the coefficient of variation is calculated by the equation: 100×standard deviation of particle size/average particle size.

In the present invention, the coefficient of variation indicating variation of the particle size is not particularly limited, but is usually 20% or less, preferably 5 to 15%.

The inventive luminescent nanoparticle according to the present invention contains a first luminescent compound and a second luminescent compound as essential components. The luminescent nanoparticle according to the present invention preferably further includes a binder as an optional component. Hereinafter, each constituting element of the luminescent nanoparticle according to the present invention will be described in order.

### <First Luminescent Compound>

The first luminescent compound is a luminescent compound a content of which to the total amount of the inventive luminescent nanoparticle is in the range from 4 to 90% by mass. The first luminescent compound has a property of absorbing light to be excited. The second luminescent compound receives the excited energy to emit light. The first luminescent compound has the maximum absorption wavelength exhibiting the maximum absorption degree for a predetermined excitation light, at which the excitation energy becomes the maximum, within a range of 4 to 90% by mass of a content of the first luminescent compound to the total amount of the luminescent nanoparticle according to the present invention. In other words, a maximum efficiency of energy transfer from excited molecules. it is preferable that the first luminescent compound is a compound in which efficiency for transferring energy from the excited molecule becomes the maximum.

It is preferable that a content of the first luminescent compound to a total amount of the luminescent nanoparticle according to the present invention is in the range of 4 to 90% by mass, and it is more preferable that it is in the range of 10 to 80% by mass.

λₑₘ₁ of the first luminescent compound is not particularly limited, but in view of the relations with the maximum absorption wavelength λ_{ab2} of the second luminescent compound and the maximum luminescence wavelength λₑₘ₂, for example, when λₑₘ₂ is in the near-infrared region, it is preferable that λₑₘ₂ is in the range of 500-900 nm, and it is more preferable that it is in the range of 600-800 nm. The maximum absorption wavelength of the first luminescent compound is denoted as λ_{ab1}. λ_{ab1} is not particularly limited, but for example, when λₑₘ₁ is within the above range, it is preferable that λ_{ab1} is in the range of 500-700 nm, and it is more preferable that it is in the range of 550-650 nm.

Preferred specific examples of the first luminescent compound according to the present invention will be described below, but these compounds may further have a substituent or may have a structural isomer or the like, and are not limited to the compounds exemplified below.

It is preferable that the first luminescent compound has a structure represented by general formula (1), general formula (2), or general formula (3) below. Hereinafter, a compound having a structure represented by the general formula (1) is also referred to as a compound (1). The same applies to other compounds.

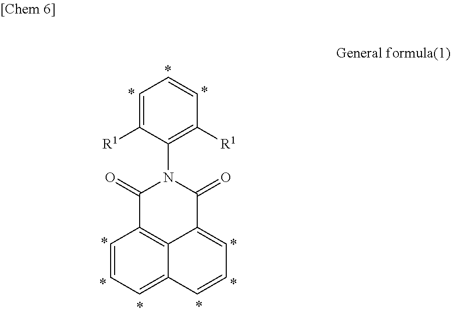

In the general formula (1), each of the plurality of R¹ independently represents a hydrogen atom or a substituent, and at least one of them represents a monovalent organic group having 3 to 30 carbon atoms. The benzene ring or the naphthalene ring may further have a substituent, and * represents a position of the substituent which may be included in the benzene ring or the naphthalene ring.

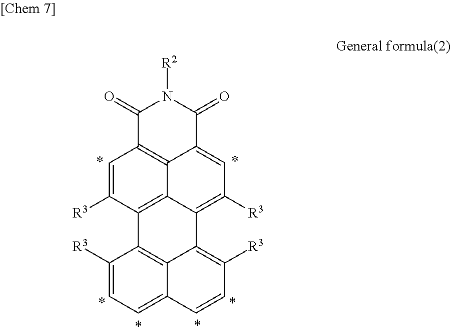

In the general formula (2), R² represents a substituted or unsubstituted alkyl group, an aryl group or a heteroaryl group. Each of the plurality of R³ independently represents a hydrogen-atom or a group having a structure represented by the following general formula (F1), and at least one of them represents a group having a structure represented by the following general formula (F1). The naphthalene ring may further have a substituent, and * represents a position of the substituent that may be included in the naphthalene ring.

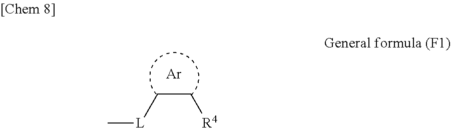

In the general formula (F1), Ar represents an aryl ring or a heteroaryl ring. R⁴ represents a substituent. When two or more groups represented by the general formula (F1) are included, the two R⁴ may be linked to each other. L represents a single bond, an oxygen-atom, a sulfur-atom or —NR′—. R′ represents a hydrogen atom, an alkyl group, an aryl group or a heteroaryl group.

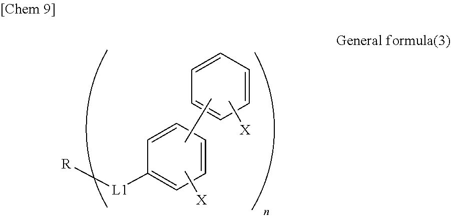

In the general formula (3), R represents a luminescent compound skeleton. Each X independently represents an ionic substituent. L1 represents a single bond, an oxygen-atom, a sulfur-atom, a selenium-atom or a NH group. n represents an integer of 1 or more.

### (Compound (1))

The compound (1) is an imide derivative having a structure represented by the following general formula (1).

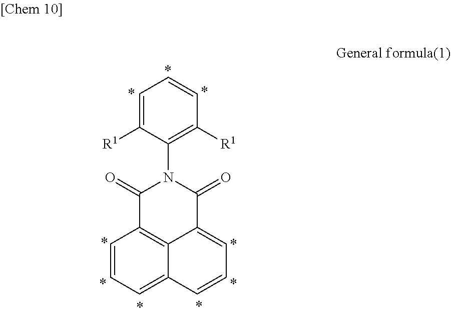

In the general formula (1), each of the plurality of R¹ independently represents a hydrogen atom or a substituent, and at least one of them represents a monovalent organic group having 3 to 30 carbon atoms. The benzene ring or the naphthalene ring may further have a substituent, and * represents a position of the substituent which may be included in the benzene ring or the naphthalene ring. In the compound (1), the substituent which may be in the position indicated by * is not particularly limited.

Specifically, it may be alkyl groups (e.g., methyl, ethyl, propyl, isopropyl, tert-butyl, pentyl, hexyl, octyl, dodecyl, tridecyl, tetradecyl, pentadecyl, etc.), cycloalkyl group (e.g., cyclopentyl group, cyclohexyl group, etc.), alkenyl group (e.g., vinyl group, allyl group, etc.), alkynyl group (e.g., ethynyl group, propargylic group, etc.), aryl group (e.g., phenyl group, p-chlorophenyl group, mesityl group, tolyl group, xylyl group, naphthyl group, anthryl group, azulenyl group, acenaphthenyl group, fluorenyl group, phenanthryl group, indenyl group, pyrenyl group, biphenylyl group, etc.), heteroaryl groups (e.g., pyridyl, pyrimidinyl, furyl, pyrrolyl, imidazolyl, benzoimidazolyl, pyrazolyl, pyrazinyl, triazolyl groups (such as 1,2,4-triazol-1 1,2,4-triazol-1-yl group, 1,2,3-triazol-1-yl group, etc.), pyrazolotriazolyl group, oxazolyl group, benzoxazolyl group, thiazolyl group, isoxazolyl group, isothiazolyl group, frazanyl group, thienyl group, quinolyl group, benzofuryl group, dibenzofuryl group, benzothienyl group, dibenzothienyl group, indolyl group, carbazolyl group, carbolynyl group, diazacarbazolyl group (one of the carbon atoms constituting the carboline ring of the above carbolynyl group is replaced by a nitrogen atom), quinoxalinyl group, pyridazinyl group, triazinyl group, quinazolinyl group, phthalazinyl group, etc.), heterocyclic groups (e.g., pyrrolidyl, imidazolidyl, morphoryl, oxazolidyl, etc.), alkoxy group (e.g., methoxy group, ethoxy group, propyloxy group, pentyloxy group, hexyloxy group, octyloxy group, dodecyloxy group, etc.), cycloalkoxy group (e.g., cyclopentyloxy group, cyclohexyloxy group, etc.), aryloxy group (e.g., phenoxy group, naphthyloxy group, etc.), alkylthio group (e.g., methylthio group, ethylthio group, propylthio group, pentylthio group, hexylthio group, octylthio group, dodecylthio group, etc.), cycloalkylthio group (e.g., cyclopentylthio group, cyclohexylthio group, etc.), arylthio group (e.g., phenylthio group, naphthylthio group, etc.), alkoxycarbonyl group (e.g., methyloxycarbonyl group, ethyloxycarbonyl group, butyloxycarbonyl group, octyloxycarbonyl group, dodecyloxycarbonyl group, etc.), aryloxycarbonyl group (e.g., phenyloxycarbonyl group, naphthyloxycarbonyl group, etc.), sulfamoyl group (e.g., aminosulfonyl group, methylaminosulfonyl group, dimethylaminosulfonyl group, butylaminosulfonyl group, hexylaminosulfonyl group, cyclohexylaminosulfonyl group, octylaminosulfonyl group, dodecylaminosulfonyl group, (phenylaminosulfonyl group, naphthylaminosulfonyl group, 2-pyridylaminosulfonyl group, etc.), acyl groups (e.g., acetyl group, ethyl carbonyl group, propyl carbonyl group, pentyl carbonyl group, cyclohexyl carbonyl group, octyl carbonyl group, 2-ethylhexyl carbonyl group, dodecyl carbonyl group, phenyl carbonyl group naphthylcarbonyl group, pyridylcarbonyl group, etc.), acyloxy group (e.g., acetyloxy group, ethylcarbonyloxy group, butylcarbonyloxy group, octylcarbonyloxy group, dodecylcarbonyloxy group, phenylcarbonyloxy group, etc.), amide group (e.g., methylcarbonylamino group, ethylcarbonylamino group, dimethylcarbonylamino group, propylcarbonylamino group, pentylcarbonylamino group, cyclohexylcarbonylamino group, 2-ethylhexylcarbonylamino group, octylcarbonylamino group, dodecylcarbonylamino group, phenylcarbonylamino group, naphthylcarbonylamino group, etc.), carbamoyl group (e.g., aminocarbonyl group, methylaminocarbonyl group, dimethylaminocarbonyl group, propylaminocarbonyl group, pentylaminocarbonyl group, cyclohexylaminocarbonyl group, octylaminocarbonyl group, 2-ethylhexylaminocarbonyl group, dodecylaminocarbonyl group, phenylaminocarbonyl group, naphthylaminocarbonyl group, 2-pyridylaminocarbonyl group, etc.), ureido group (e.g., methylureido group, ethylureido group, pentylureido group, cyclohexylureido group, octylureido group, dodecylureido group, phenylureido group naphthylureido group, 2-pyridylamino-ureido group, etc.), sulfinyl groups (e.g., methyl sulfinyl, ethyl sulfinyl, butyl sulfinyl, cyclohexyl sulfinyl, 2-ethyl hexyl sulfinyl, dodecyl sulfinyl, phenyl sulfinyl, naphthyl sulfinyl, 2-pyridylsulfinyl group, etc.), alkylsulfonyl group (e.g., methylsulfonyl group, ethylsulfonyl group, butylsulfonyl group, cyclohexylsulfonyl group, 2-ethylhexylsulfonyl group, dodecylsulfonyl group, etc.), arylsulfonyl or heteroarylsulfonyl group (e.g., phenylsulfonyl group, naphthylsulfonyl group, 2-pyridylsulfonyl group, etc.), amino group (e.g., amino group, ethylamino group, dimethylamino group, diphenylamino group, diisopropylamino group, ditertbutyl group, cyclohexylamino group, butylamino group, cyclopentylamino group, 2-ethylhexylamino group, dodecylamino group, anilino group, naphthylamino group, 2-pyridylamino group, etc.), halogen atom (e.g., fluorine atom, chlorine atom, bromine atom, etc.), fluorinated hydrocarbon group (e.g., fluoromethyl group, trifluoromethyl group, pentafluoroethyl group, pentafluorophenyl group, etc.), cyano group, nitro group, hydroxy group, mercapto group, silyl group (e.g., trimethylsilyl group, triisopropylsilyl group, triphenylsilyl group, phenyldiethylsilyl group, etc.), phosphono group, carboxy group, sulfo group.

These substituents may be further substituted by the above-described substituents. Furthermore, these substituents may be bonded to each other to form a ring. The cyclic structure formed by the adjacent substituents may be an aromatic ring or an aliphatic ring, or may contain a hetero atom, and the cyclic structure may be a fused ring of two or more rings.

Preferably, the position of * does not have a substituent, or the substituent is an alkyl group, a halogen atom, a cyano group, a carboxylic anhydride in which two carboxylic acids are condensed, or a condensed ring in which substituents are bonded to each other.

R¹ independently represents a hydrogen atom or a substituent, and at least one of them represents a group having 3 to 30 carbon atoms. The substituent represented by R¹ can be specifically selected from the above-mentioned substituents which may be included in *, and at least one of the substituents is a group having 3 to 30 carbon atoms. The group having 3 to 30 carbon atoms allows R¹ of the ortho-position substituent to effectively shield the x-plane because the steric hindrance between the carbonyl group of the imide and R¹ orients the nitrogen-substituted phenyl group perpendicular to the naphthalene ring.

It is also preferred that R¹ has oxygen- or sulfur-atoms in the carbon-chain. It is more preferred to have oxygen atoms in the carbon chain. The presence of oxygen- or sulfur-atoms in the carbon-chain provides a more flexible conformation and can increase shielding effect of the π-plane by R¹.

R¹ is preferably an alkyl group (e.g., n-propyl, isopropyl, n-butyl, sec-butyl, isobutyl, tert-butyl, pentyl, hexyl, octyl, dodecyl, tridecyl, tetradecyl, pentadecyl, 3-ethylpentyl, etc.), cycloalkyl group (e.g., cyclopentyl, cyclohexyl, cyclohexylethyl, etc.), alkenyl group (e.g., propenyl group, hexenyl group, etc.), alkynyl group (e.g., propynyl, hexynyl, phenylethynyl, etc.), aryl group (e.g., phenyl, p-chlorophenyl, mesityl, tolyl, xylyl, naphthyl, anthryl, azulenyl, acenaphthenyl, fluorenyl, phenanthryl, indenyl, pyrenyl, biphenylyl, etc.), heteroaryl group (e.g., pyridyl group, pyrimidinyl group, furyl group, pyrrolyl group, benzoimidazolyl group, pyrazolyl group, pyrazinyl group, benzoxazolyl group, thienyl group, quinolyl group, benzofuryl group, dibenzofuryl group, benzothienyl group, dibenzothienyl group, indolyl group carbazolyl group, carbolynyl group, diazacarbazolyl group (one of the carbon atoms constituting the carboline ring of the above carbolynyl group is replaced by a nitrogen atom), quinoxalinyl group, pyridazinyl group, triazinyl group, quinazolinyl group, phthalazinyl group, etc.), heterocyclic groups (e.g., pyrrolidyl, imidazolidyl, morphoryl, oxazolidyl, etc.), alkoxy group (e.g., pentyloxy group, hexyloxy group, octyloxy group, dodecyloxy group, 2-ethylbutyloxy group, etc.), cycloalkoxy group (e.g., cyclopentyloxy group, cyclohexyloxy group, etc.), aryloxy group (e.g., phenoxy group, naphthyloxy group, etc.), alkylthio group (e.g., pentylthio group, hexylthio group, octylthio group, dodecylthio group, etc.), cycloalkylthio group (e.g., cyclopentylthio group, cyclohexylthio group, etc.), arylthio group (e.g., phenylthio group, naphthylthio group, etc.), alkoxycarbonyl group (e.g., butyloxycarbonyl group, octyloxycarbonyl group, dodecyloxycarbonyl group, etc.), aryloxycarbonyl group (e.g., phenyloxycarbonyl group, naphthyloxycarbonyl group, etc.), sulfamoyl group (e.g., butylaminosulfonyl group, hexylaminosulfonyl group, cyclohexylaminosulfonyl group, octylaminosulfonyl group, dodecylaminosulfonyl group, phenylaminosulfonyl group, naphthylaminosulfonyl group, 2-pyridylaminosulfonyl group, etc.), acyl groups (e.g., butylcarbonyl group, pentylcarbonyl group, cyclohexylcarbonyl group, octylcarbonyl group, 2-ethylhexylcarbonyl group, dodecylcarbonyl group, phenylcarbonyl group, naphthylcarbonyl group, pyridylcarbonyl group, etc.), acyloxy group (e.g., butylcarbonyloxy group, octylcarbonyloxy group, dodecylcarbonyloxy group, phenylcarbonyloxy group, etc.), amide group (e.g., propylcarbonylamino group, pentylcarbonylamino group, cyclohexylcarbonylamino group, 2-ethylhexylcarbonylamino group, octylcarbonylamino group, dodecylcarbonylamino group, phenylcarbonylamino group, (naphthylcarbonylamino group, etc.), carbamoyl group (e.g., diethylaminocarbonyl group, propylaminocarbonyl group, pentylaminocarbonyl group, cyclohexylaminocarbonyl group, octylaminocarbonyl group, 2-ethylhexylaminocarbonyl group, dodecylaminocarbonyl group Phenylaminocarbonyl group, naphthylaminocarbonyl group, 2-pyridylaminocarbonyl group, etc.), ureido group (e.g., pentylureido group, cyclohexylureido group, octylureido group, dodecylureido group, phenylureido group naphthylureido group, 2-pyridylamino-ureido group, etc.), sulfinyl groups (e.g., butyl sulfinyl, cyclohexyl sulfinyl, 2-ethylhexyl sulfinyl, dodecyl sulfinyl, phenyl sulfinyl, naphthyl sulfinyl, 2-pyridyl sulfinyl, etc.), alkylsulfonyl group (e.g., butylsulfonyl group, cyclohexylsulfonyl group, 2-ethylhexylsulfonyl group, dodecylsulfonyl group, etc.), arylsulfonyl or heteroarylsulfonyl group (e.g., phenylsulfonyl group, naphthylsulfonyl group, 2-pyridylsulfonyl group, etc.), amino group (e.g., diphenylamino group, diisopropylamino group, cyclohexylamino group, butylamino group, cyclopentylamino group, 2-ethylhexylamino group, dodecylamino group, anilino group, naphthylamino group, 2-(pyridylamino group, etc.) pyridylamino group, etc.) fluorocarbon group (e.g., decafluorobutyl group, pentafluorophenyl group, etc.) silyl group (e.g., triethylsilyl group, triisopropylsilyl group, triphenylsilyl group, phenyldiethylsilyl group, etc.).

R¹ is more preferably a bulky group, and examples thereof include an alkyl group containing an aryl group, a heteroaryl group, and a secondary or higher carbon (for example, a secondary carbon: an isobutyl group, a cyclohexyl group, a cyclopentyl group, a cholesteryl group, a tertiary carbon: a tert-butyl group, an adamantyl group, a [2,2,2] bicyclooctyl group, and the like), a tertiary amino group (for example, a diethylamino group, a diphenylamino group, and the like), and a tertiary silyl group (for example, a triisopropylsilyl group, a triphenylsilyl group, a phenyldiethylsilyl group, and the like). The alkyl group, alkenyl group, alkynyl group, alkoxy group, acyl group, acyloxy group, and amide group may have such a bulky group at the end.

The compound (1) is preferably a compound having a structure represented by any one of the following general formulae (2-1) to (2-6).

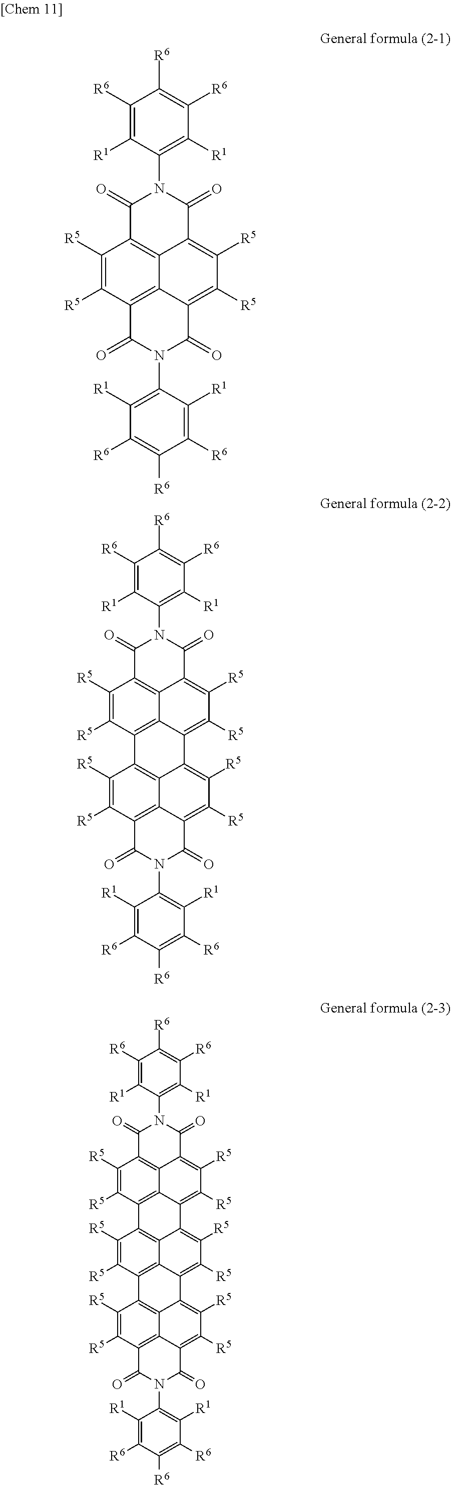

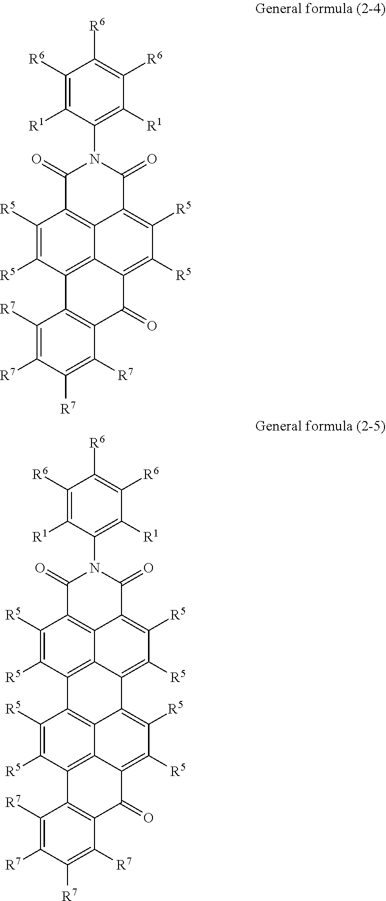

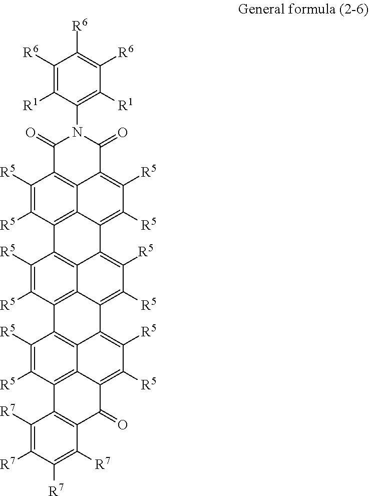

(In the formulas, a plurality of R¹ each independently represents a hydrogen atom or a substituent, and at least one of them represents a group having a carbon number of 3 to 30. R⁵, R⁶ and R⁷ each independently represents a hydrogen atom, an alkyl group, an aryl group, a heteroaryl group, an alkynyl group, an alkoxy group, or an aryloxy group.)

R¹ has the same meaning as R¹ in the general formula (1).

R⁵, R⁶ and R⁷ each independently represent a hydrogen-atom, an alkyl-group, an aryl-group, a heteroaryl-group, an alkenyl-group, an alkynyl-group, an alkoxy-group, or an aryloxy-group.

These groups have the same meaning as the alkyl group, aryl group, heteroaryl group, alkenyl group, alkynyl group, alkoxy group or aryloxy group as the substituent which * may have in the general formula (1).

According to the present invention, a perylene bisimide derivative having a structure represented by the general formula (2-2) is preferred, and the perylene bisimide derivative is preferably a compound (31) having a structure represented by the following general formula (31). The perylene bisimide derivative is desirable because it not only exhibits high luminesce quantum yield but also exhibits high light resistance.

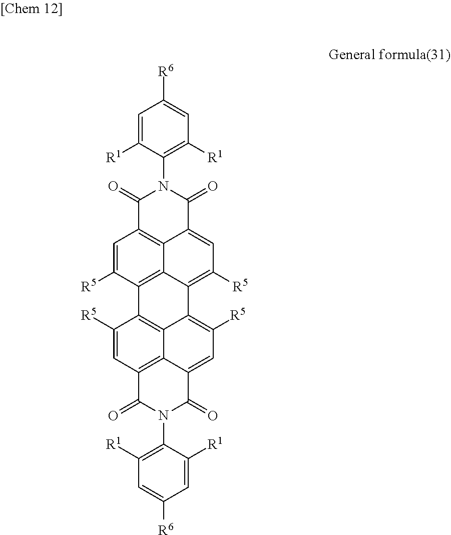

In the general formula (31), a plurality of R¹ each independently represents a hydrogen atom or a substituent, and at least one of them represents a group having 3 to 30 carbon atoms. Each R⁵ independently represents a hydrogen-atom, an alkyl-group, an aryl-group, a heteroaryl-group, an alkenyl-group, an alkynyl-group, an alkoxy-group, or an aryloxy-group. R⁶ independently represents a hydrogen-atom, an alkyl-group, an aryl-group, a heteroaryl-group, an alkenyl-group, an alkynyl-group, an alkoxy-group, or an aryloxy-group.

In the general formula (31), R⁵ is preferably a phenoxy group or a group represented by the following general formula (2-2-1) (hereinafter also referred to as a group (2-2-1)).

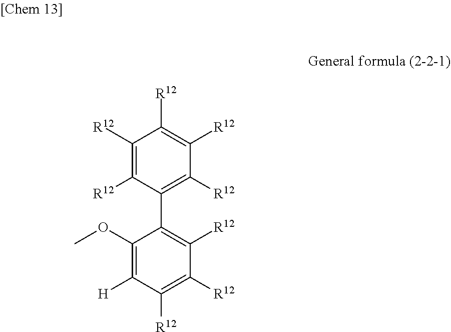

In the general formula (2-2-1). R¹² represents a hydrogen-atom or a substituent. The substituent has the same meaning as the substituent which may be contained in the position indicated by * in the general formula (1).

The compound (31) is more preferably a compound having a structure represented by the following general formula (1c), general formula (1d) or general formula (1e), or the like.

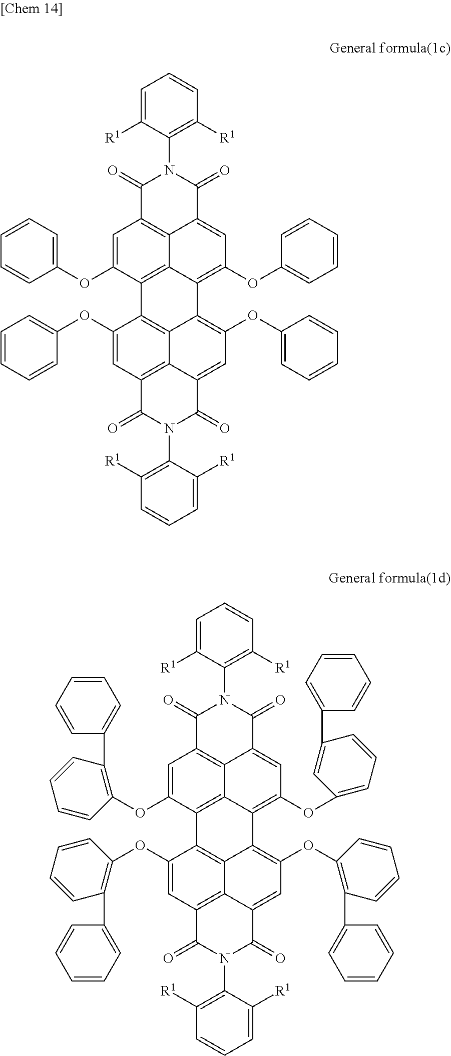

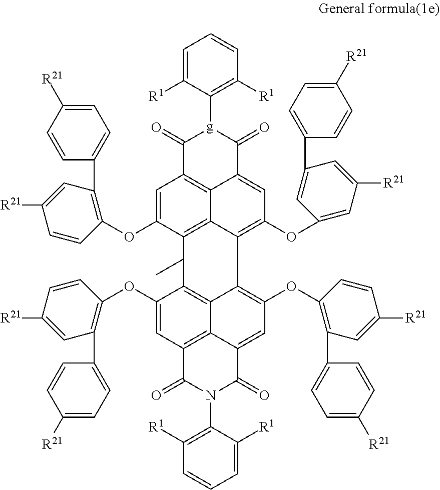

In general formulae (1c) to (1e), a plurality of R¹ each independently represents a hydrogen atom or a substituent, and at least one of them represents a monovalent organic group having 3 to 30 carbon atoms. In the general formula (1e), R²¹ independently represents a hydrogen-atom or an ionic substituent, and at least one of them represents an ionic substituent. R¹ has the same meaning as R¹ represented by the general formula (1), and specific examples thereof are as described above.

The compound (1c) is a compound in which R⁶ is a hydrogen-atom and R⁵ is a phenoxy group in the general formula (31). In the compound (1c), R⁵ serving as the bay area is a phenoxy group, and thereby, the solubility can be improved and the wavelength of λₑₘ₁ can be increased, which is preferable as the first luminescent compound.

The compound (1d) is a compound in which R⁶ is a hydrogen atom and R⁵ is a group (2-2-1) (with the proviso that all R¹² are hydrogen atoms) in the general formula (31). The compound (1d) is preferred as the first luminescent compound from the viewpoint of suppressing density quenching due to reduction of intermolecular interaction of the perylene site.

The compound (1e) is a compound in which, in the compound (1d), at least one of R¹² (R²¹ in the general formula (1e)) located at the 4-position of the benzene ring in R¹² in the group (2-2-1) is substituted with an ionic substituent. The compound (1e) is preferable as the first luminescent compound from the viewpoint of improving solubility by having an ionic substituent and suppressing density quenching due to repulsion of electrostatic force.

In the compound (1e), a compound in which all of R²¹ are substituted with an ionic substituent is also a compound (3) described later, more specifically, a compound classified as a compound (4). The ionic substituent in the compound (1e) has the same meaning as in the compound (3) described later. Specific examples of the compound (1e) are described as specific examples of the compound (3) described later.

### (Compound (2))

The compound (2) is an imide derivative having a structure represented by the following general formula (2).

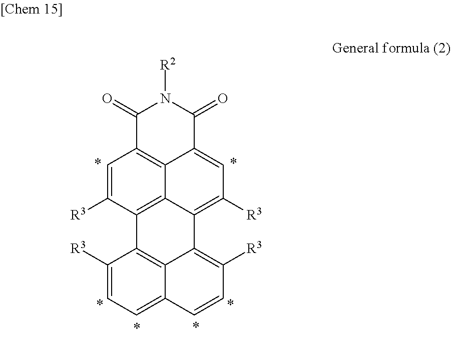

In the general formula (2), R² represents a substituted or unsubstituted alkyl group, an aryl group or a heteroaryl group. Each of the plurality of R³ independently represents a hydrogen-atom or a group having a structure represented by the following general formula (F1), and at least one of them represents a group having a structure represented by the following general formula (F1) (hereinafter also referred to as a substituent (F1)). The naphthalene ring may further have a substituent, and * represents the position of the substituent that may be included in the naphthalene ring.

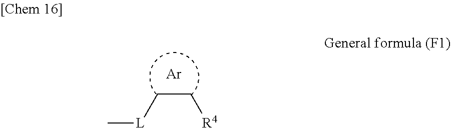

In the general formula (F1), Ar represents an aryl ring or a heteroaryl ring. R⁴ represents a substituent. When two or more groups represented by the general formula (F1) are included, the two R⁴ may be linked to each other. L represents a single bond, an oxygen-atom, a sulfur-atom or —NR′—. R′ represents a hydrogen atom, an alkyl group, an aryl group or a heteroaryl group.

In the compound (2), the ortho-substituent R⁴ of the aryl ring or the heteroaryl ring represented by Ar of the substituent (F1) is oriented toward the perylene ring and effectively shields the x-plane, so that a higher quantum yield can be exhibited.

Ar represents an aryl or heteroaryl ring that may have substituents, and examples of aryl rings include benzene, naphthalene, azulene, anthracene, phenanthrene, naphthacene, and pyrene rings.

Heteroaryl rings can include pyridine ring, pyrimidine ring, furan ring, pyrrole ring, imidazole ring, benzimidazole ring, pyrazole ring, pyrazine ring, triazole ring, pyrazolotriazole ring, oxazole ring, benzoxazole ring, thiazole ring, thiophene ring, quinoline ring, benzofuran ring, dibenzofuran ring, indole ring, quinoxaline ring, triazine ring, etc. Preferably, Ar represents an aryl ring.

R⁴ represents a substituent and may be selected from the substituents which * may have in the general formula (1).

The alkyl group, aryl group and heteroaryl group represented by R′ have the same meanings as the alkyl group, aryl group and heteroaryl group mentioned as the substituent which * may have in the general formula (1).

R⁴ is preferably an alkyl group (for example, a methyl group, an ethyl group, a propyl group, a butyl group, a pentyl group, a hexyl group, an isopropyl group, a tert-butyl group, an isobutyl group, a neopentyl group), a cycloalkyl group (for example, a cyclopentyl group, a cyclohexyl group), an aryl group (for example, a phenyl group, a naphthyl group, an anthryl group), a heteroaryl group (for example, a pyridyl group, a carbazolyl group), an alkenyl group (for example, a butenyl group, a pentenyl group, a hexenyl group), an alkynyl group (for example, a propynyl group, a hexynyl group, a phenylethynyl group, a trimethylsilylethynyl group), a silyl group (for example, a trimethylsilyl group, a triethylsilyl group, a triphenylsilyl group An alkoxy group (methoxy group, tert-butyloxy group) or an aryloxy group (phenoxy group, naphthoxy group).

R² represents a substituted or unsubstituted alkyl group, an aryl group or a heteroaryl group. R² has the same meaning as the alkyl group, the aryl group and the heteroaryl group mentioned as the substituent which * may have in the general formula (1). As R², a substituted or unsubstituted aryl group, particularly a substituted or unsubstituted phenyl group, is preferable. R² is preferably a group represented by the following general formula (F2):

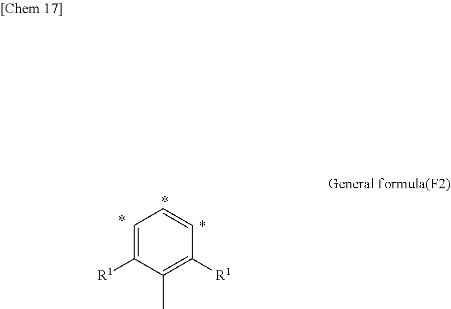

In the general formula (F2), a plurality of R¹ each independently represents a hydrogen atom or a substituent, and at least one of them represents a group having 3 to 30 carbon atoms. The benzene ring may further have a substituent, and * represents the position of the substituent which may be included in the benzene ring. The substituents which may be present at the positions indicated by R¹ and * in the general formula (F2) have the same meanings as the substituents which may be present in R¹ and * in the general formula (1), respectively.

The compound (2) preferably has a structure represented by the following general formulae (7-1) to (7-4).

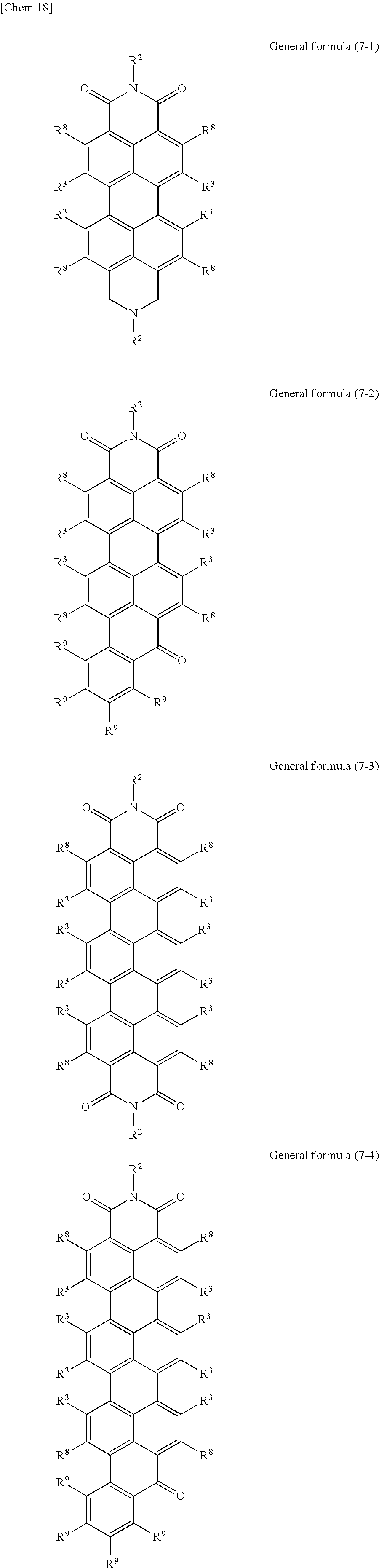

In the formulae, R² independently represents a substituted or unsubstituted alkyl group, an aryl group or a heteroaryl group. Each of the plurality of R³ independently represents a hydrogen-atom or a group having a structure represented by the general formula (6), and at least one of them represents a group having a structure represented by the general formula (6). R⁸ and R⁹ each independently represent a hydrogen-atom, an alkyl-group, an alkenyl-group, an alkynyl-group, an aryl-group, a heteroaryl-group, an alkoxy-group, or an aryloxy-group.

R² and R³ have the same meanings as R² and R³ in the general formula (2). Alkyl groups, alkynyl groups, alkynyl groups, aryls, heteroaryls, alkoxys and aryloxy groups represented by R⁸ and R⁹ are synonymous in the general formula (1) with an alkyl group, alkynyl groups, alkynyls, alkynyls, aryls, heteroaryls, alkoxys and aryloxy groups as a substituent which * may have.

According to the present invention, a perylene bisimide derivative having a structure represented by the general formula (7-1) is preferred, and the perylene bisimide derivative is preferably a compound (8) having a structure represented by the following general formula (8).

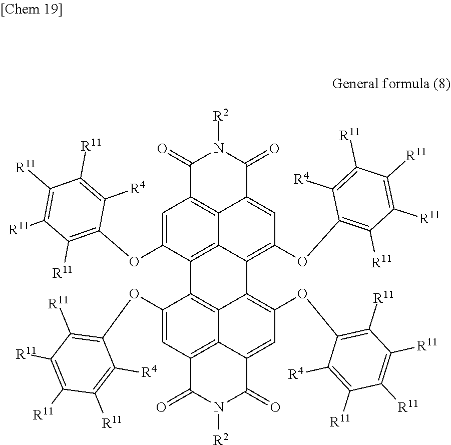

In the general formula (8), a plurality of R² each independently represents a substituted or unsubstituted alkyl group, an aryl group or a heteroaryl group. R⁴ represents a substituent. R⁴ may be connected to each other. R¹¹ represent independently hydrogen-atom, alkyl group, aryl group, heteroaryl group, alkenyl group, alkynyl group, alkoxy group, aryloxy group, amino group, acyl group, acyloxy group, amide group, carboxy group or sulfo group, respectively.)

R² and R⁴ have the same meanings as R² and R⁴ in the general formula (2), respectively. Further, the imide derivative having a structure represented by the general formula (8) is preferably an imide derivative having a structure represented by the general formula (8A).

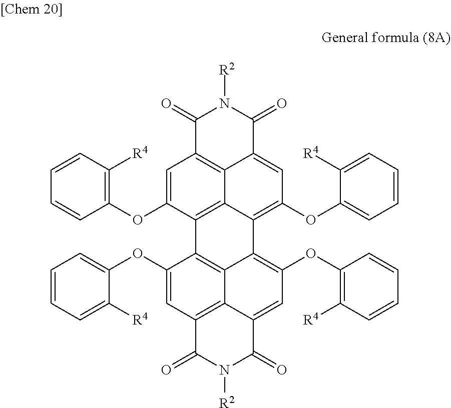

In the general formula (8A), a plurality of R² each independently represents a substituted or unsubstituted alkyl group, an aryl group, or a heteroaryl group. R⁴ represents a substituent. R⁴ may be connected to each other. R² and R⁴ have the same meanings as R² and R⁴ in the general formula (8).

The compound (8A) is a phenoxy group having a substituent R⁴ in all four bay areas, and the substituent R⁴ is preferably oriented above and below the perylene ring to enhance shielding effect.

Furthermore, in the above-mentioned general formula (8), it is preferable that any two of R⁴ are crossed and connected on the perylene. The linking effectively inhibits the interaction between the perylene rings and exhibits a higher luminesce quantum yield.

Examples of the imide derivative having a structure represented by the general formulae (1) to (8) according to the present invention are shown below, but the present invention is not limited thereto.

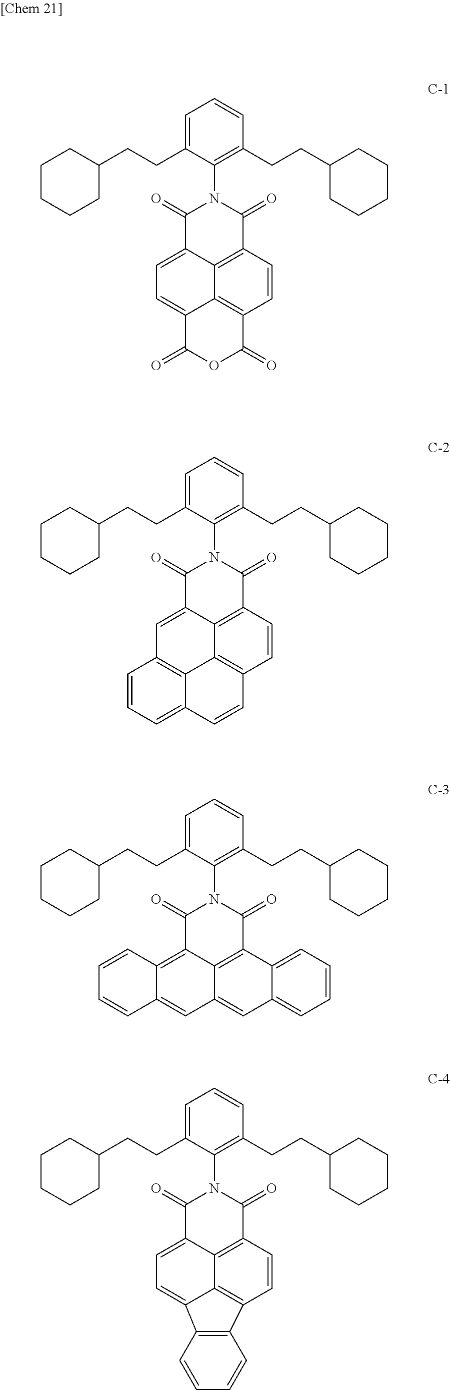

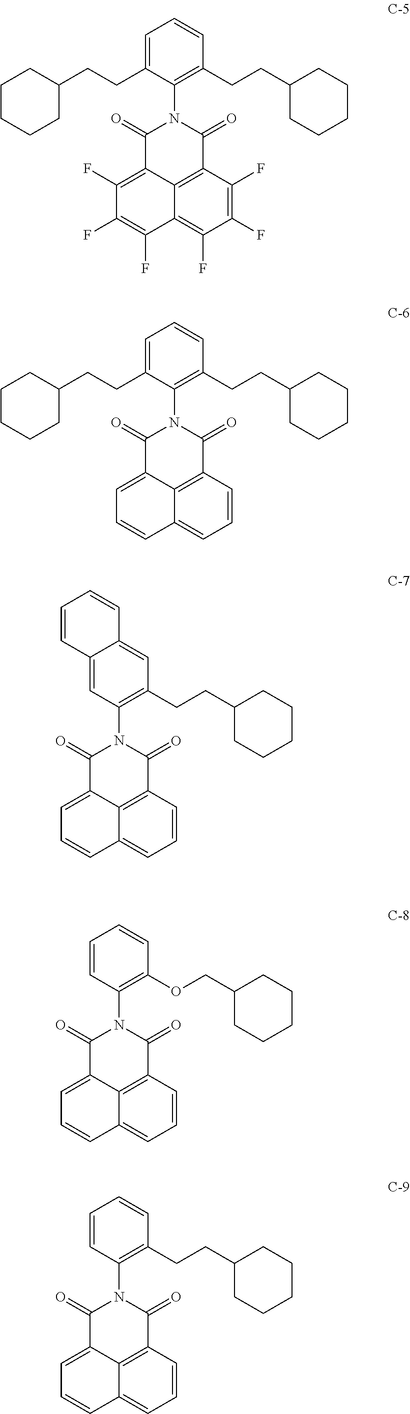

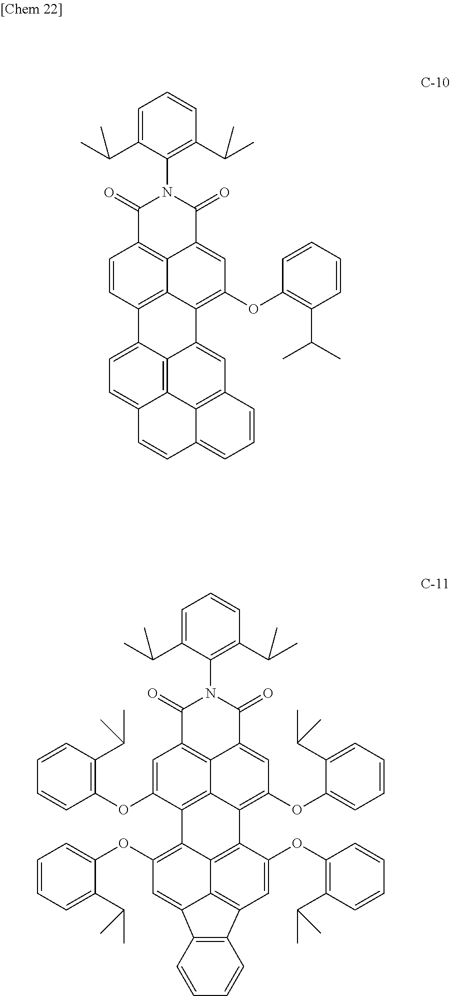

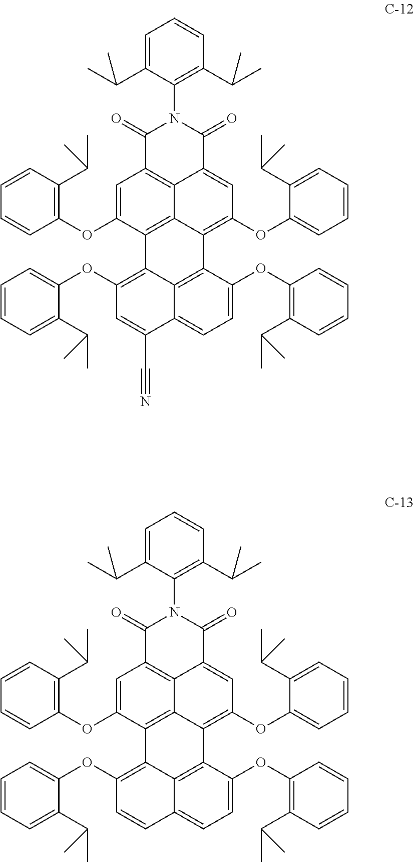

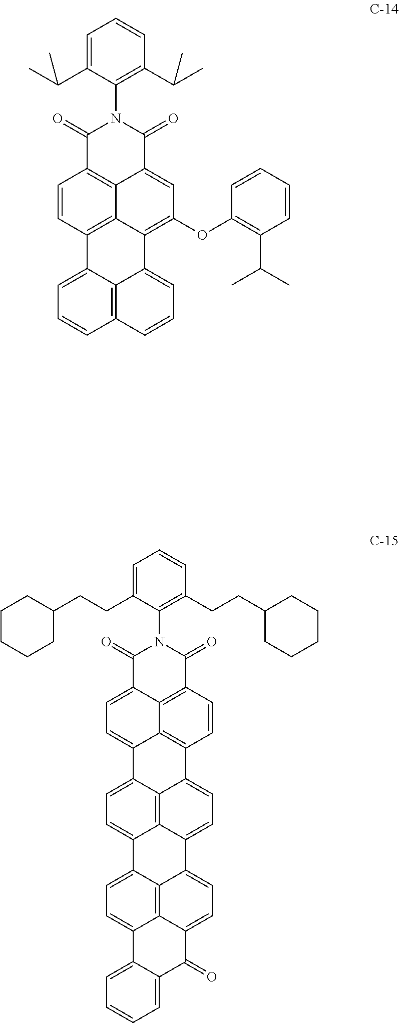

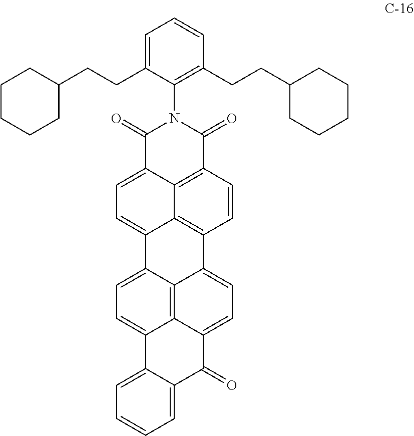

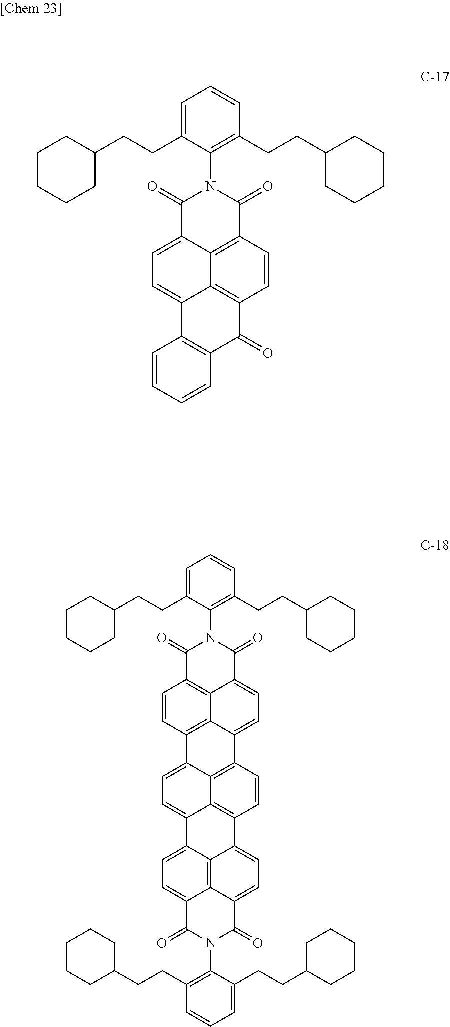

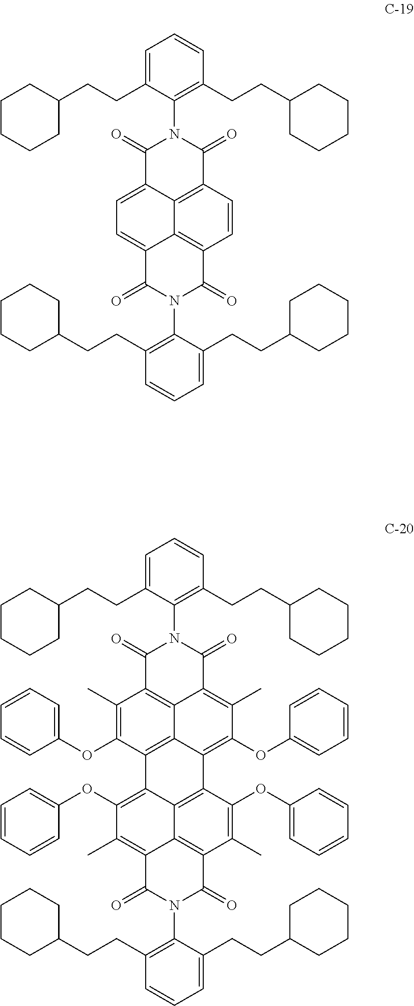

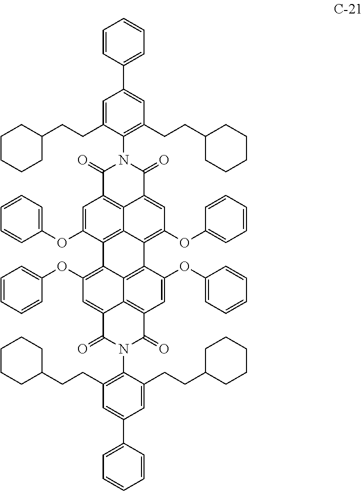

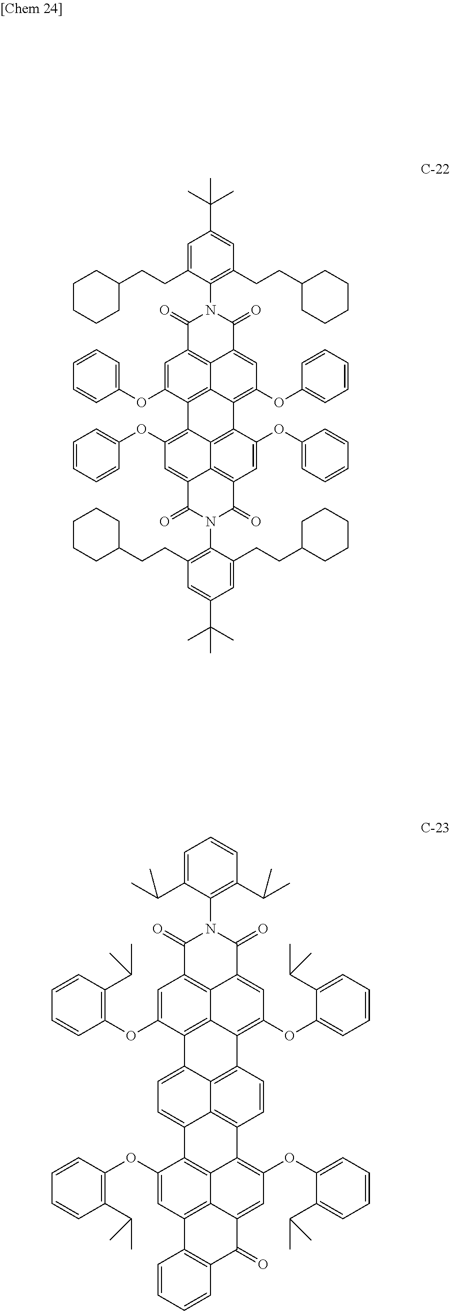

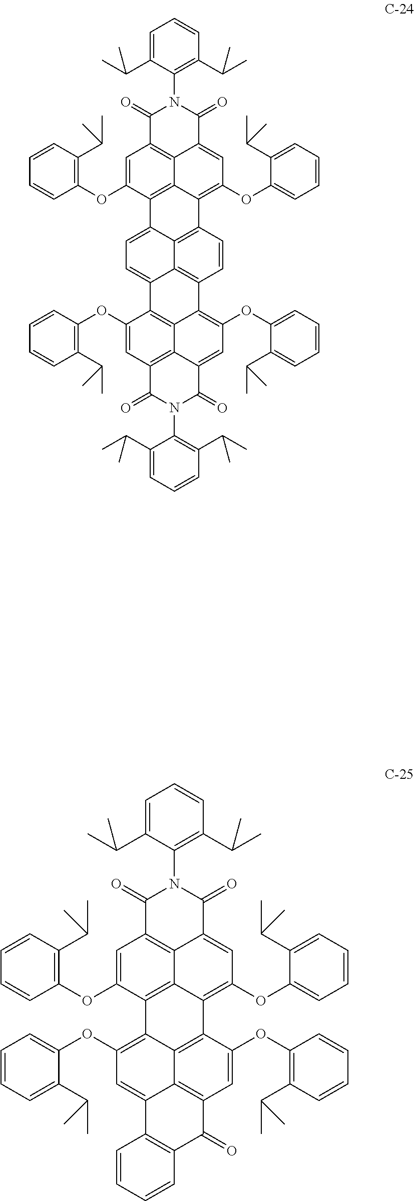

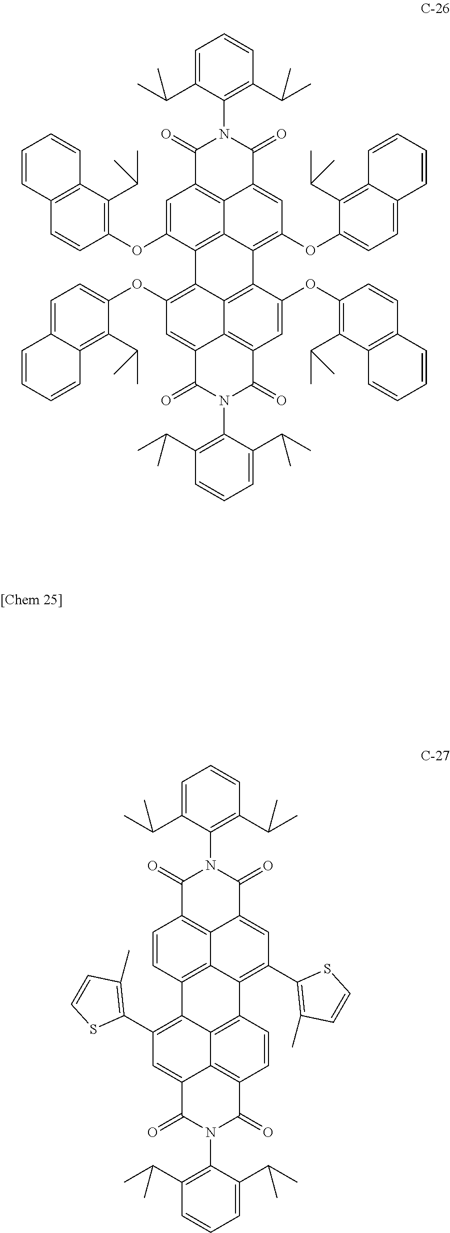

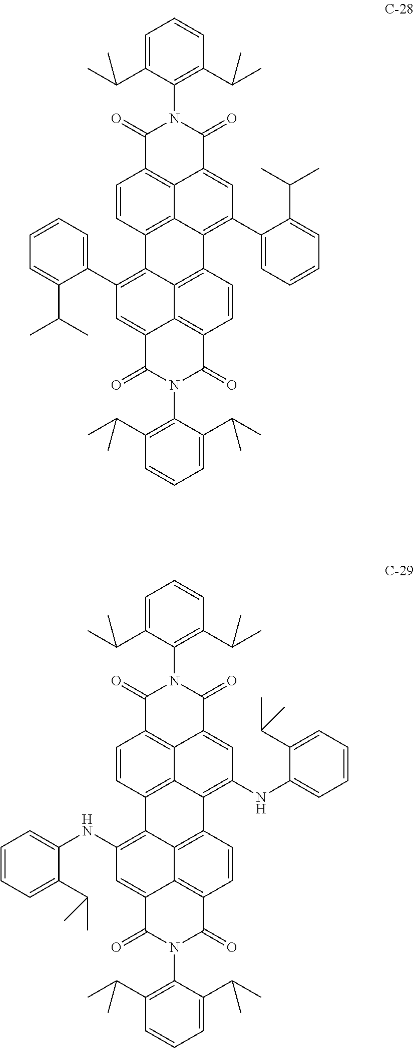

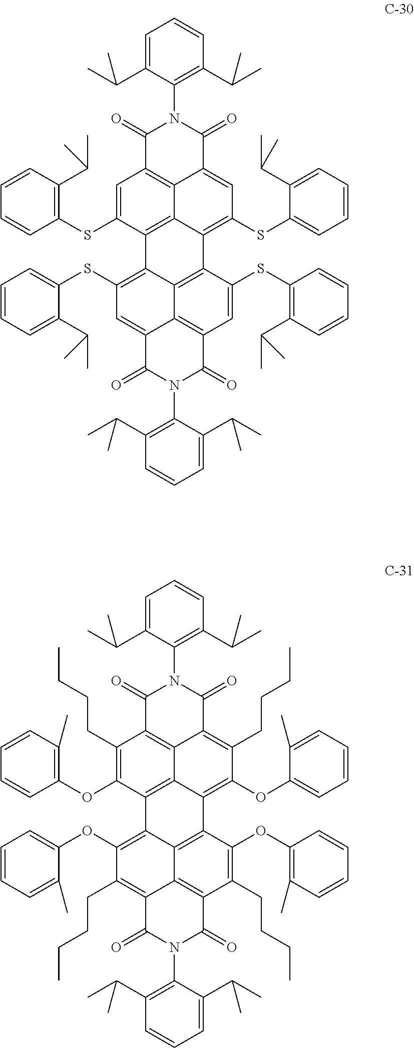

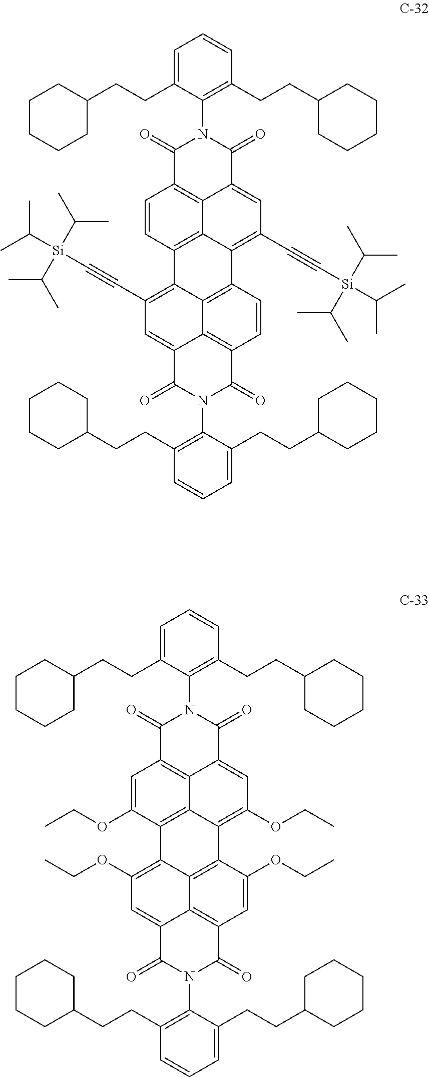

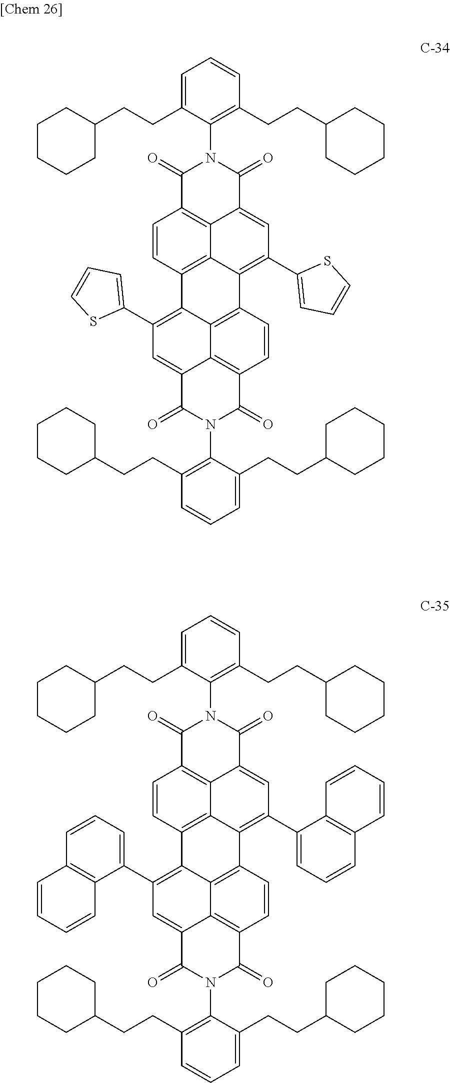

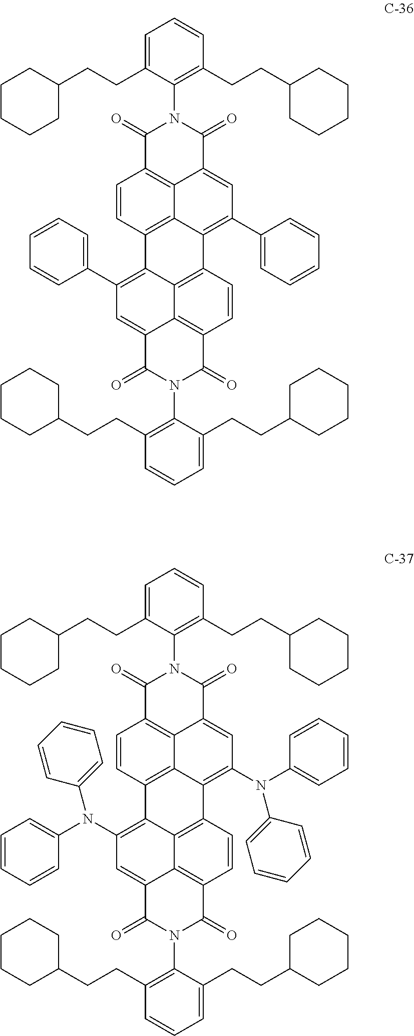

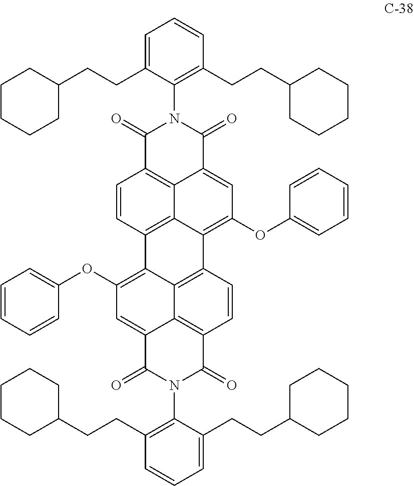

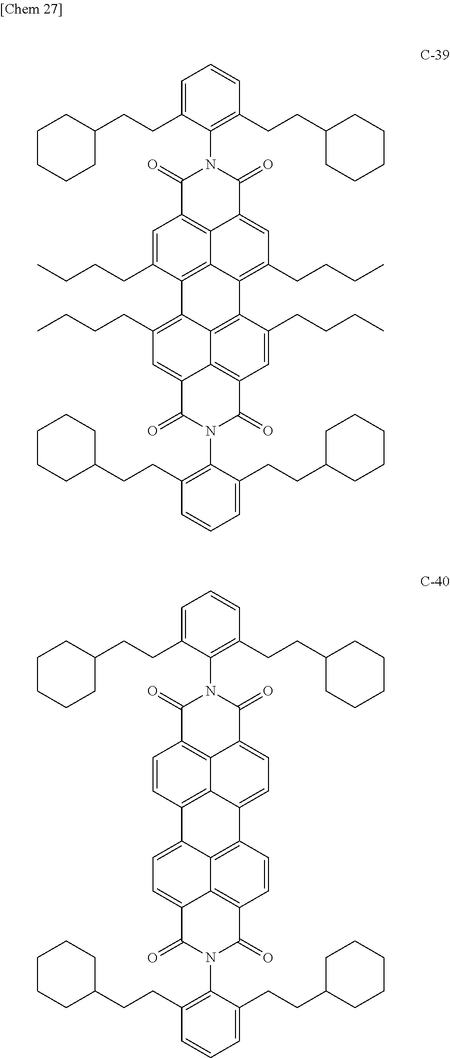

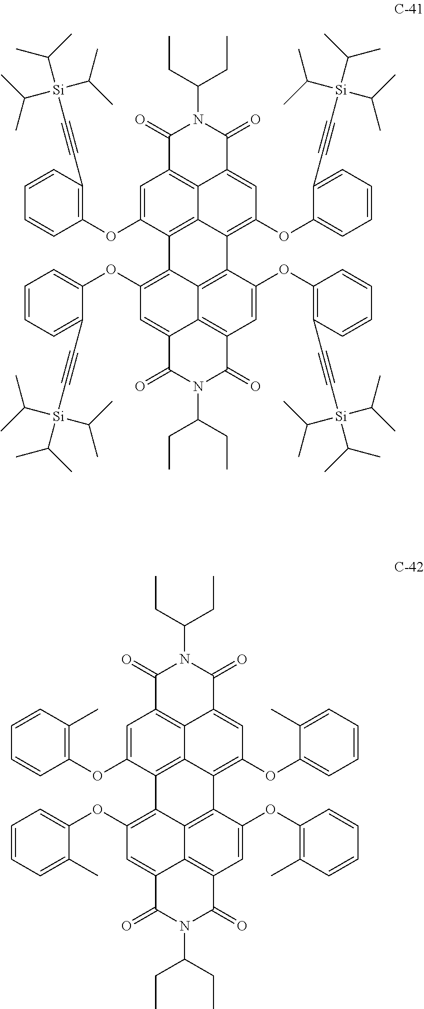

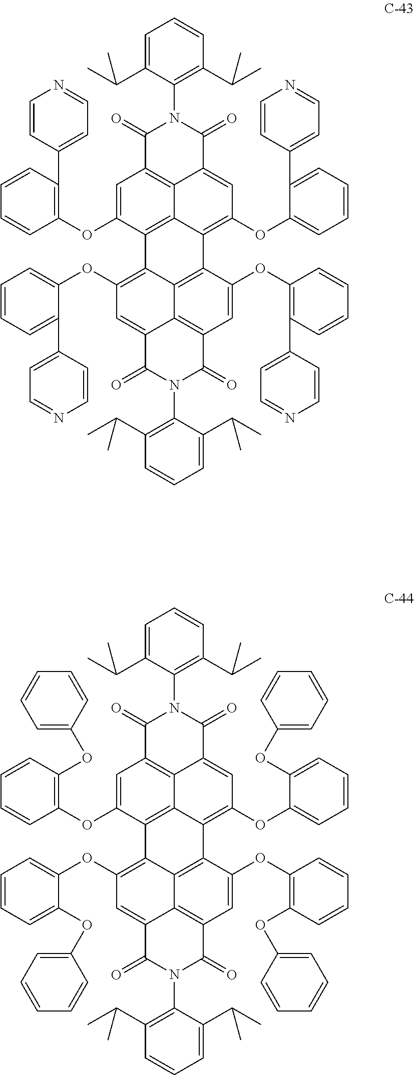

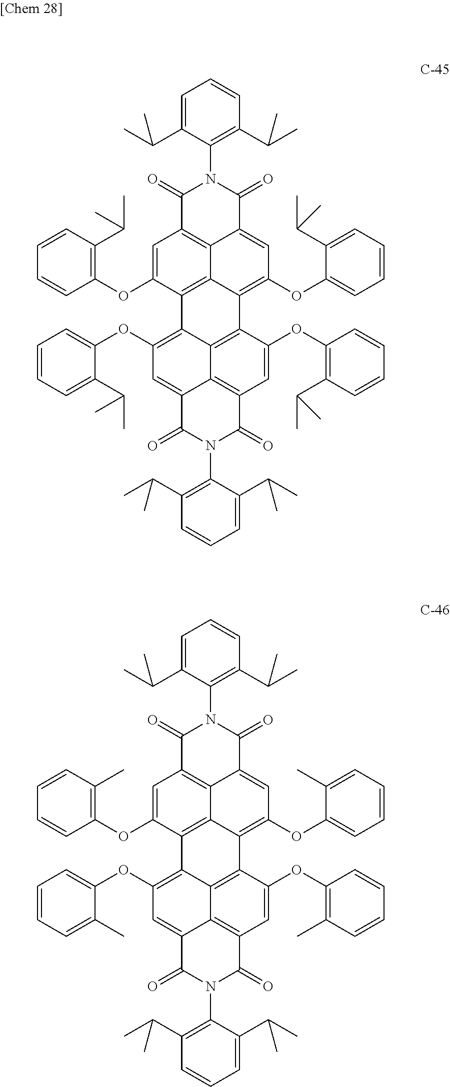

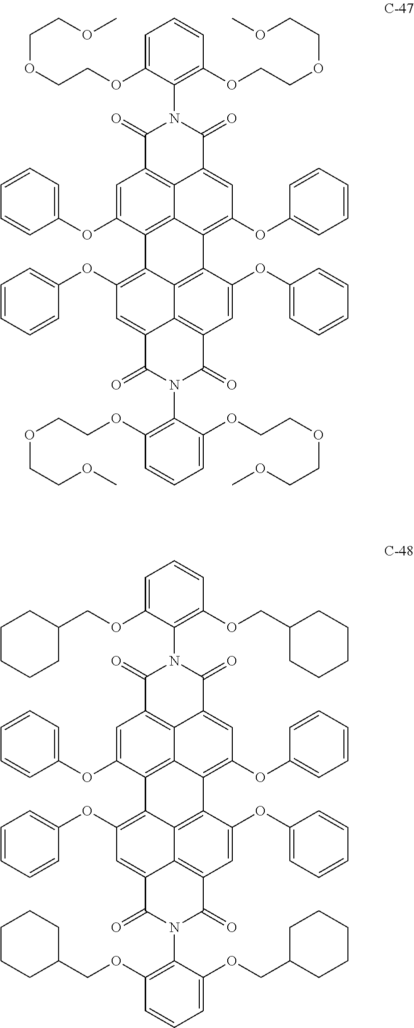

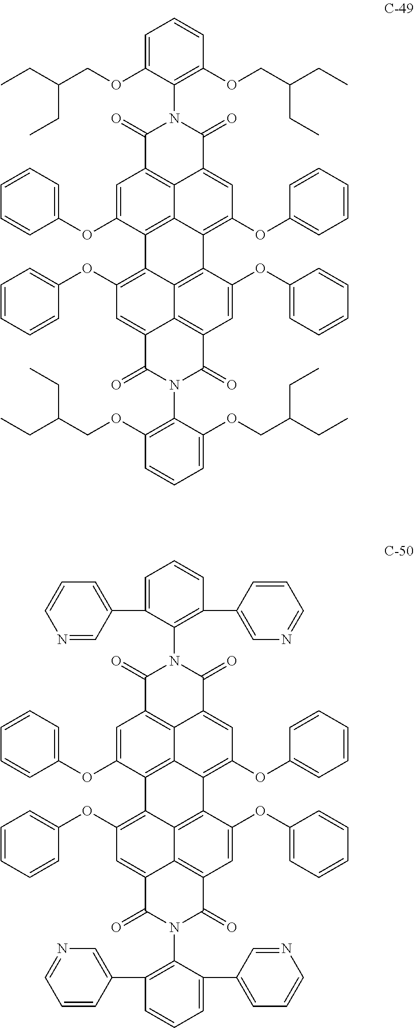

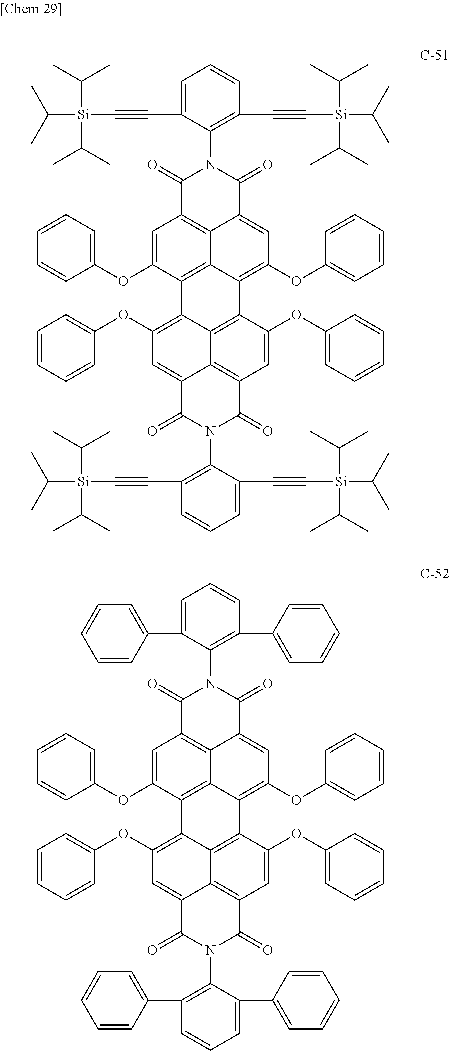

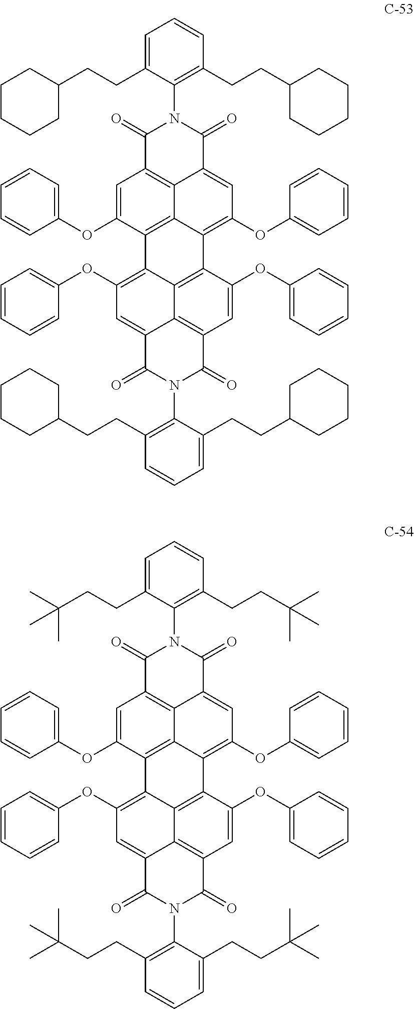

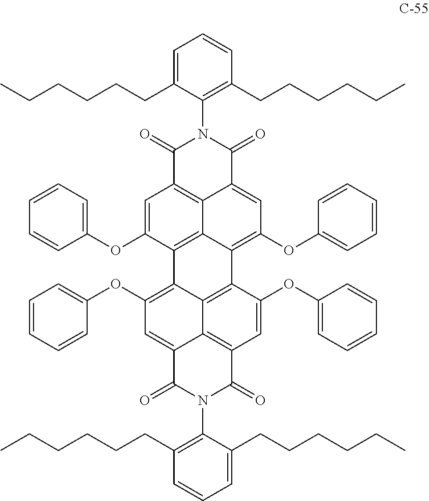

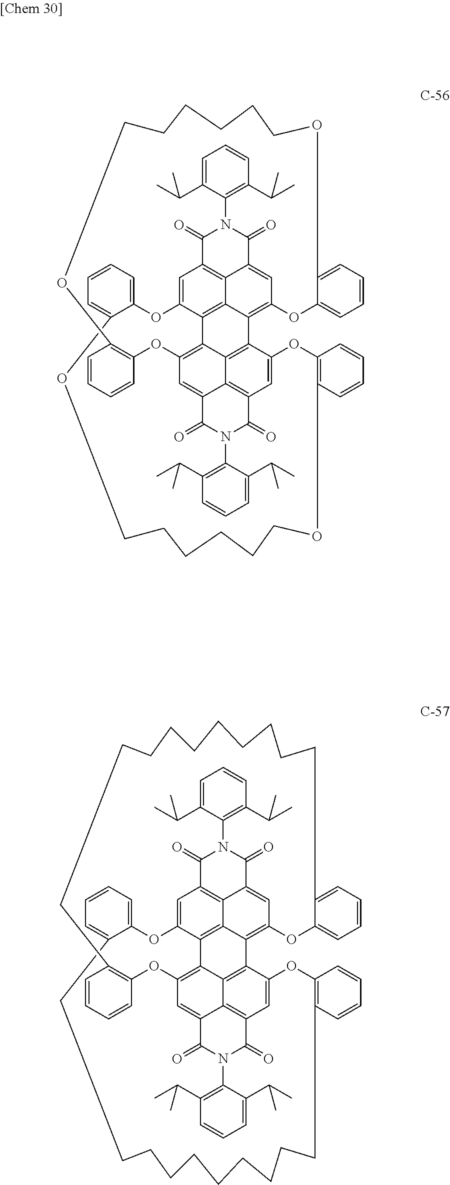

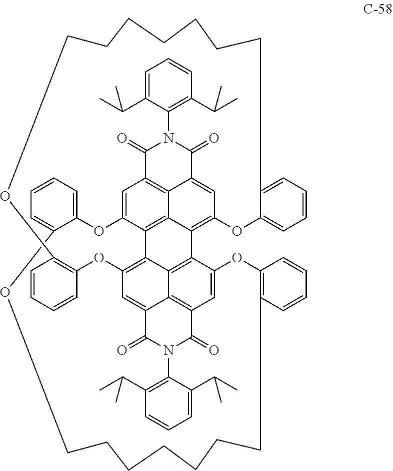

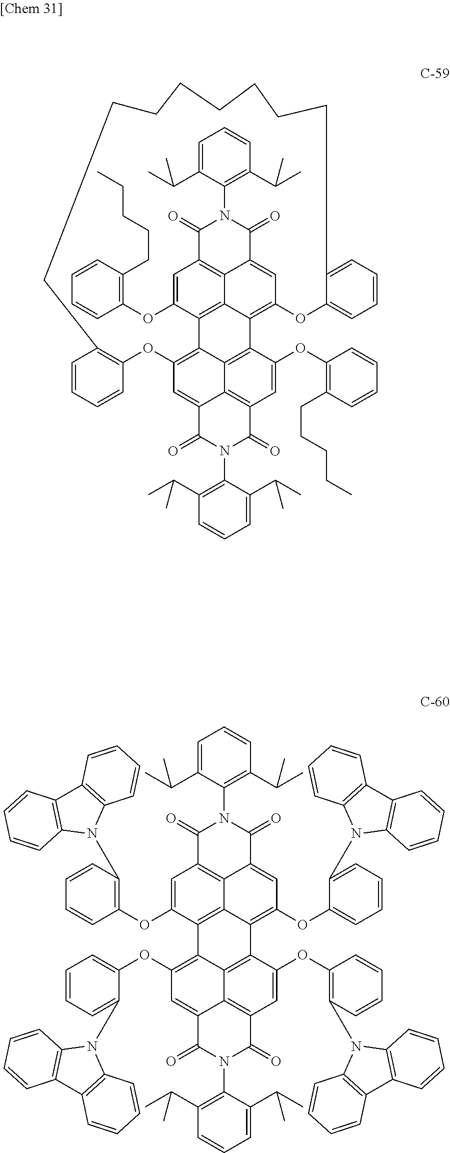

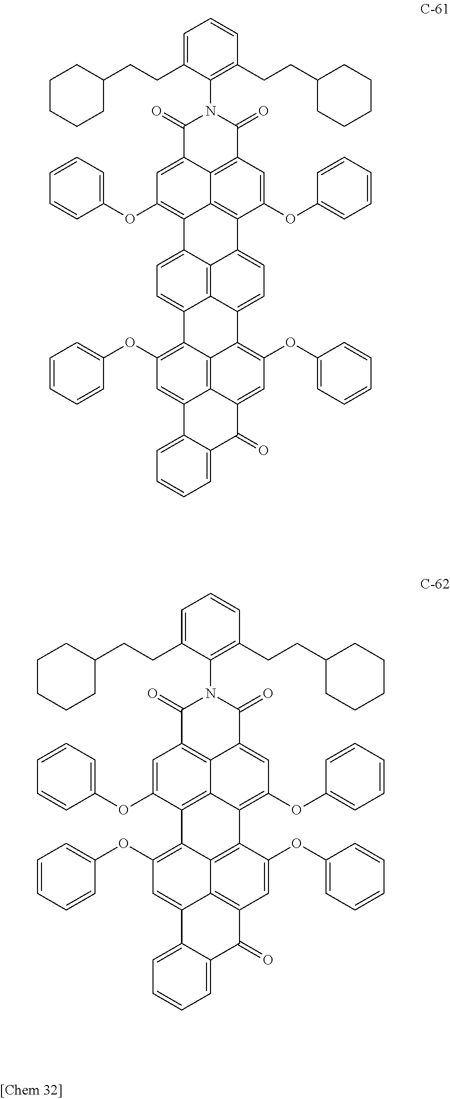

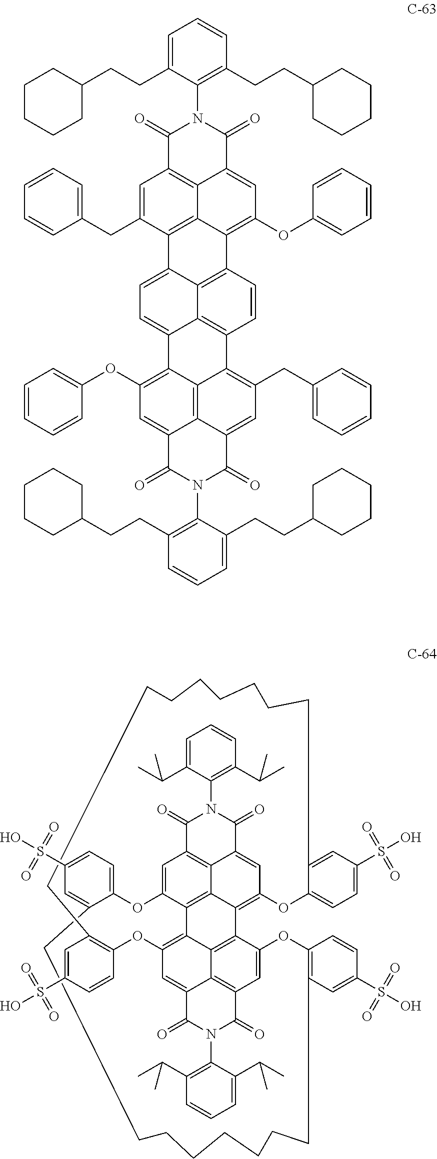

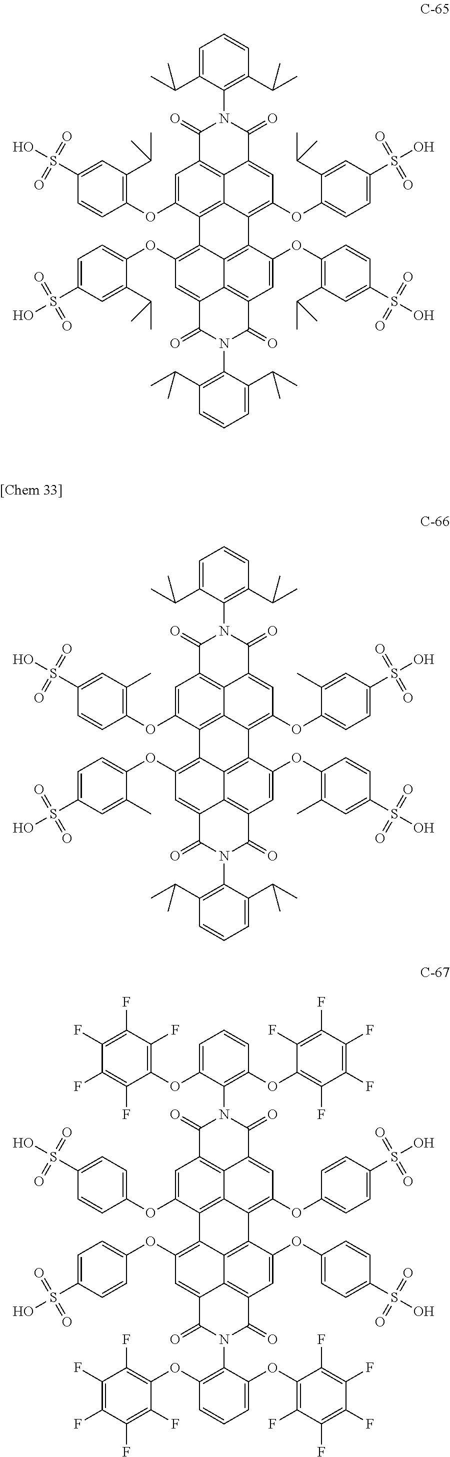

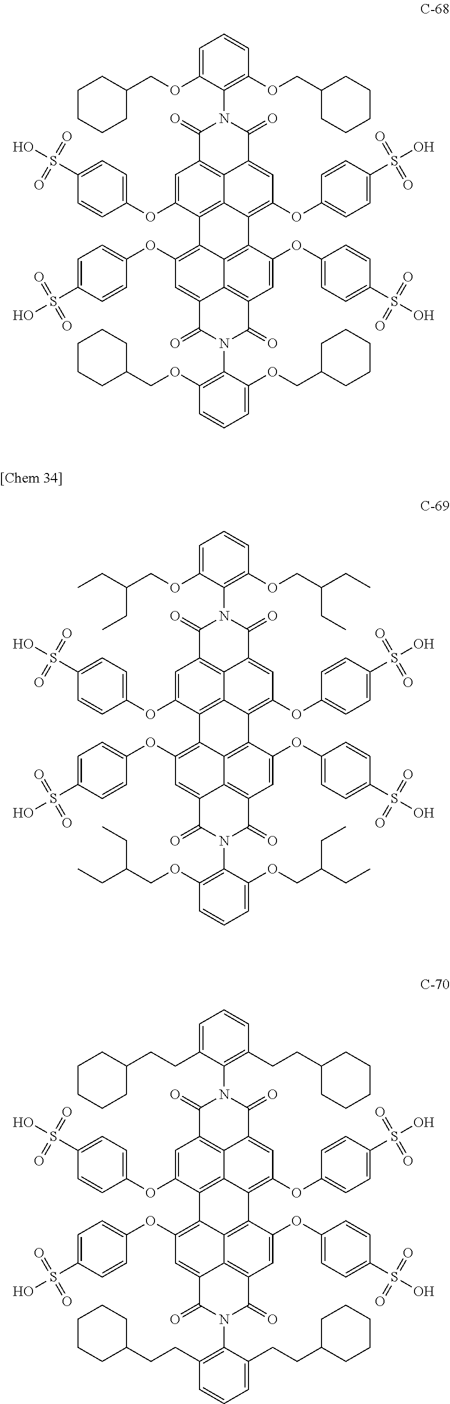

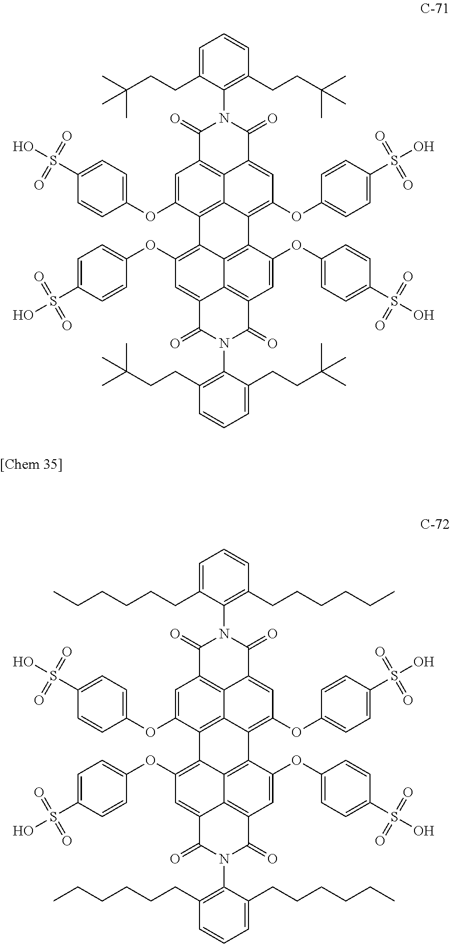

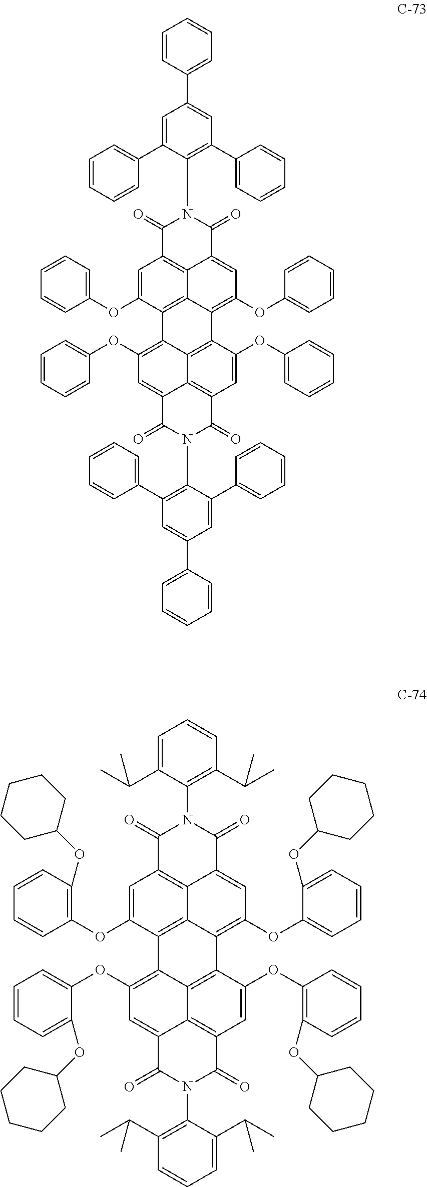

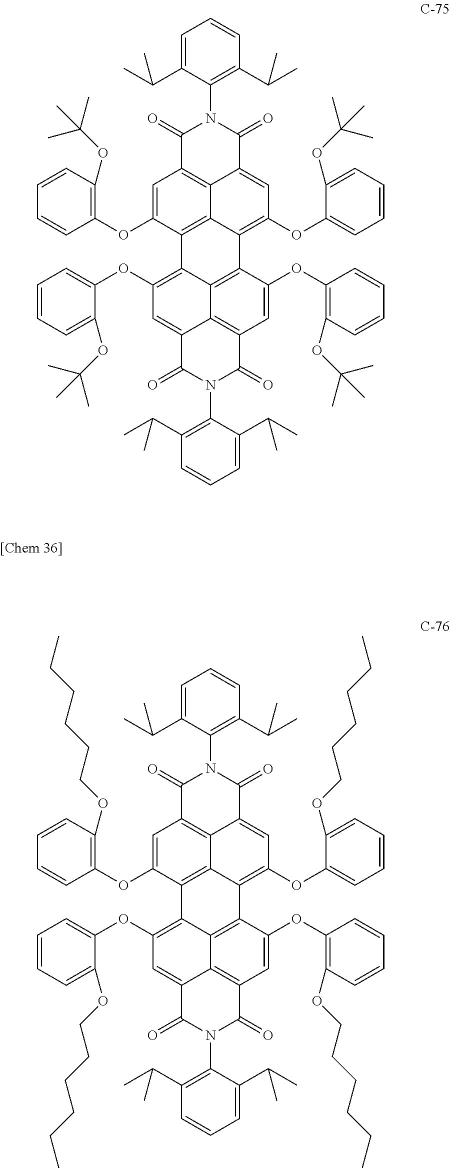

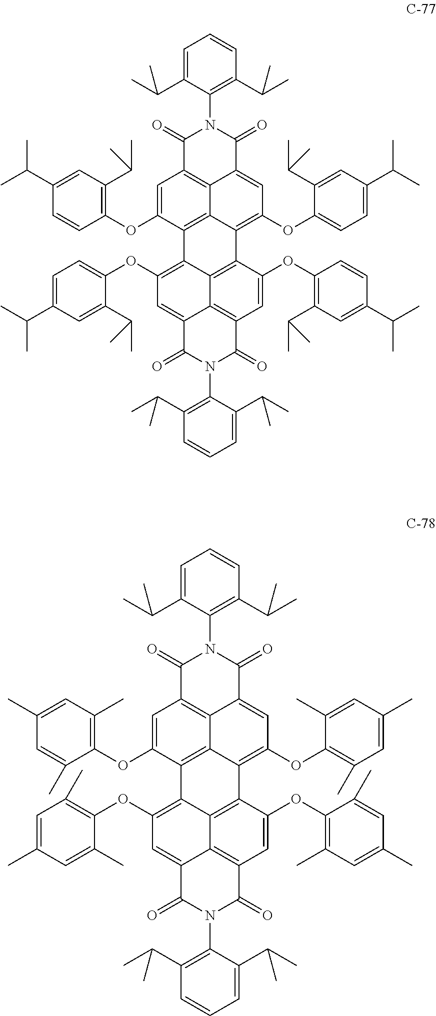

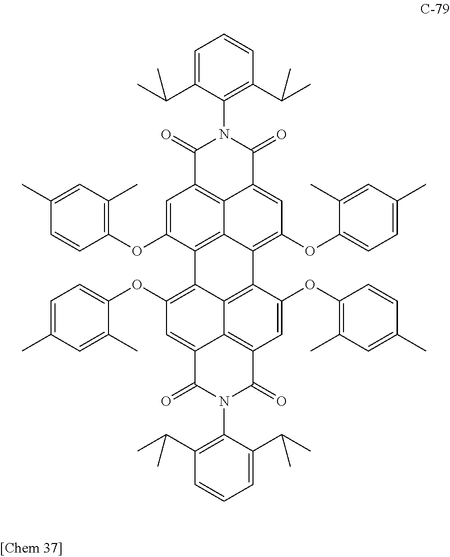

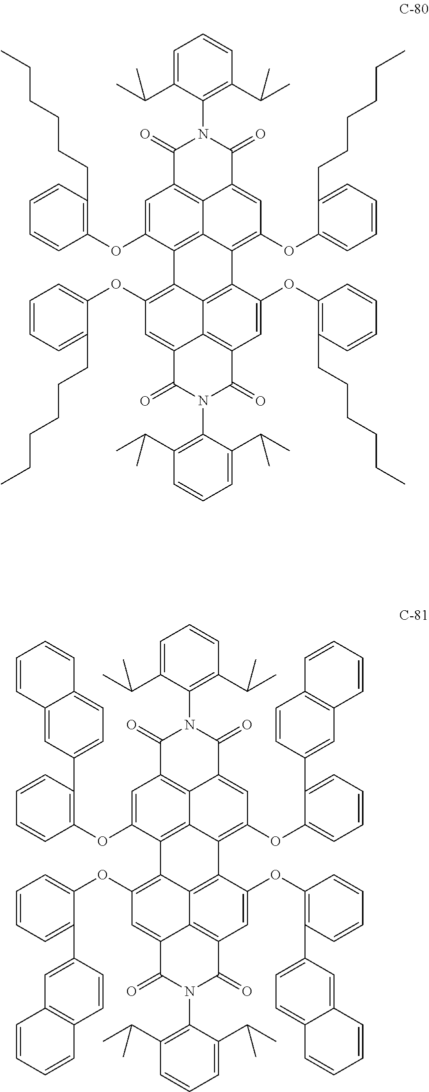

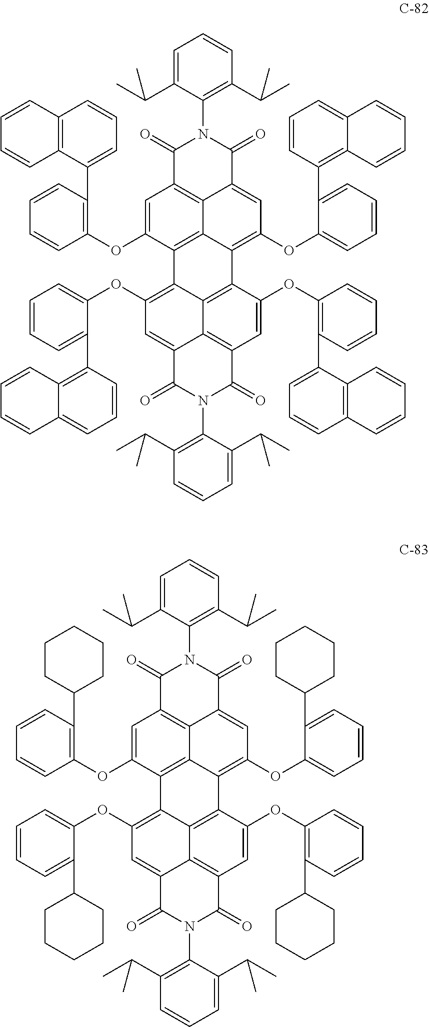

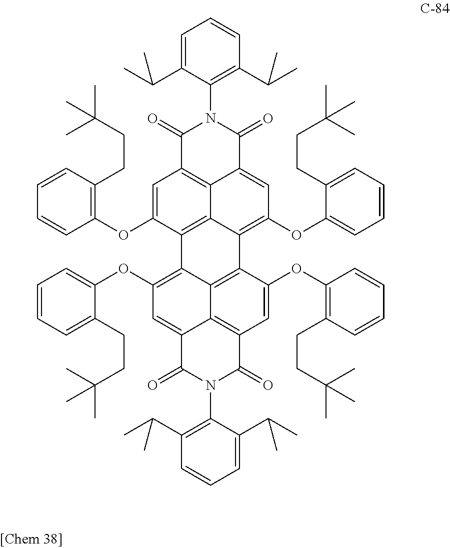

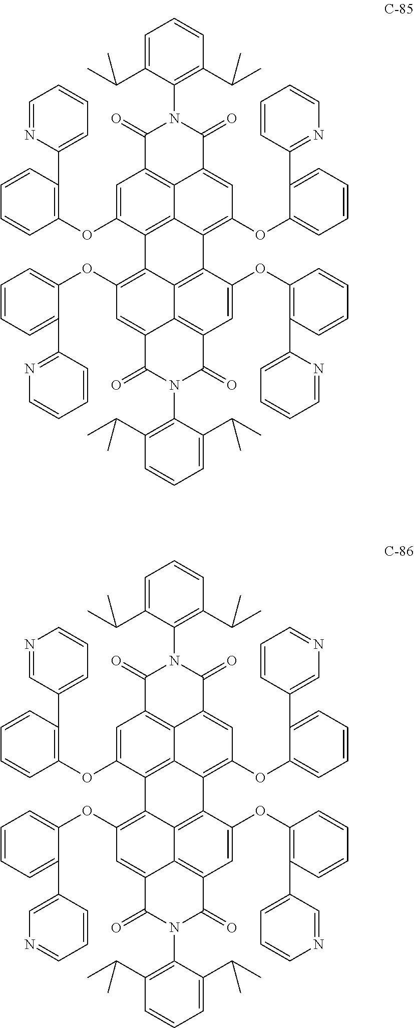

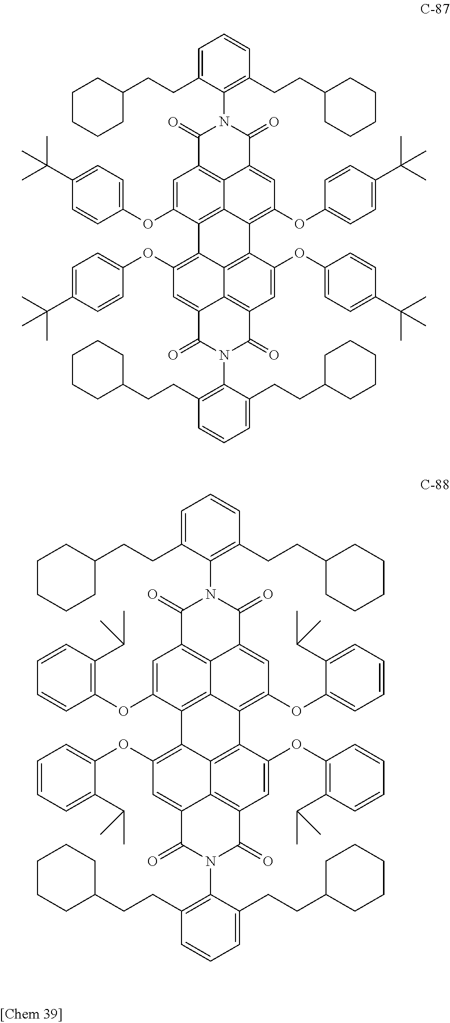

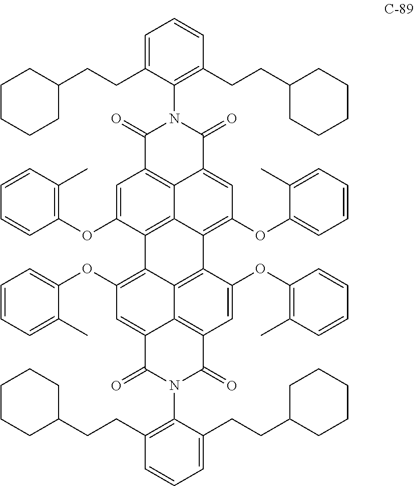

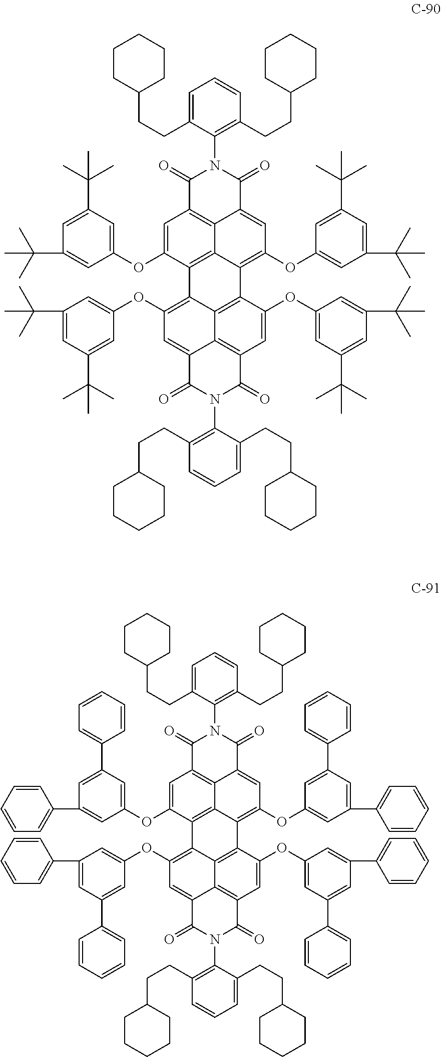

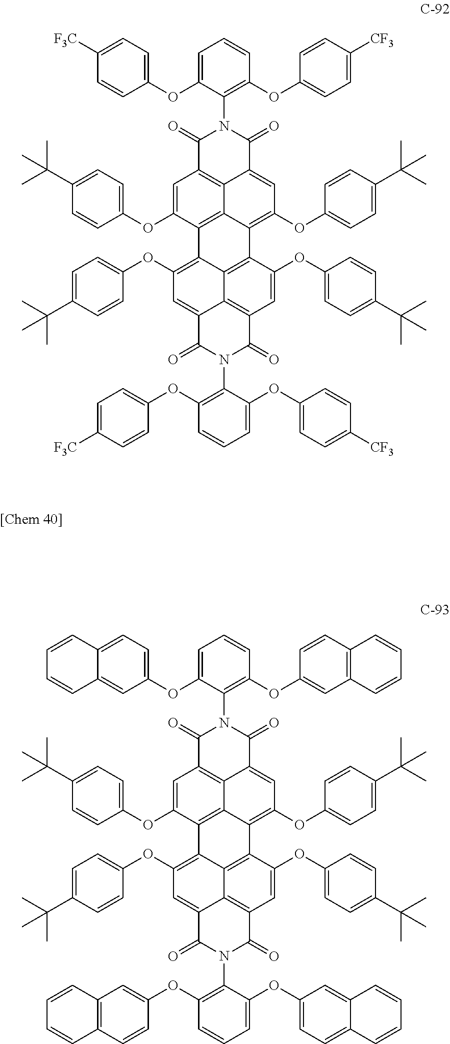

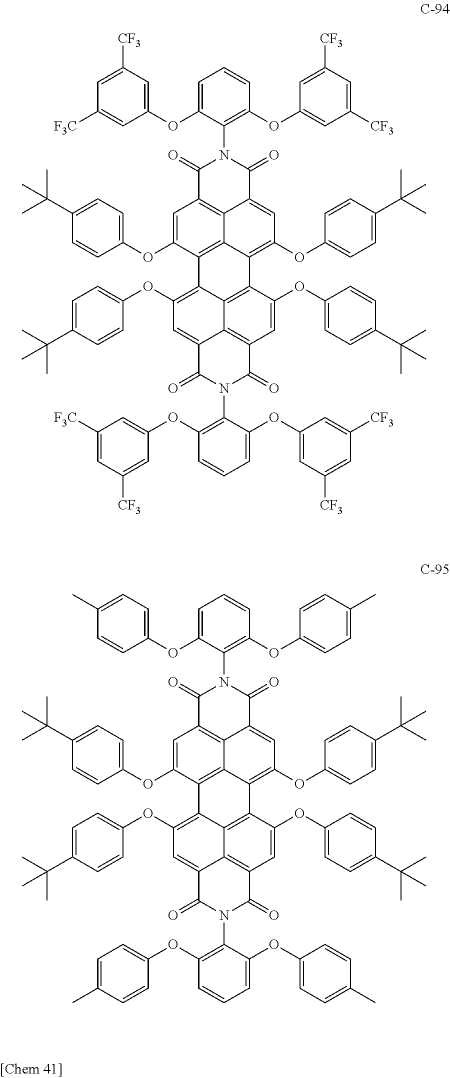

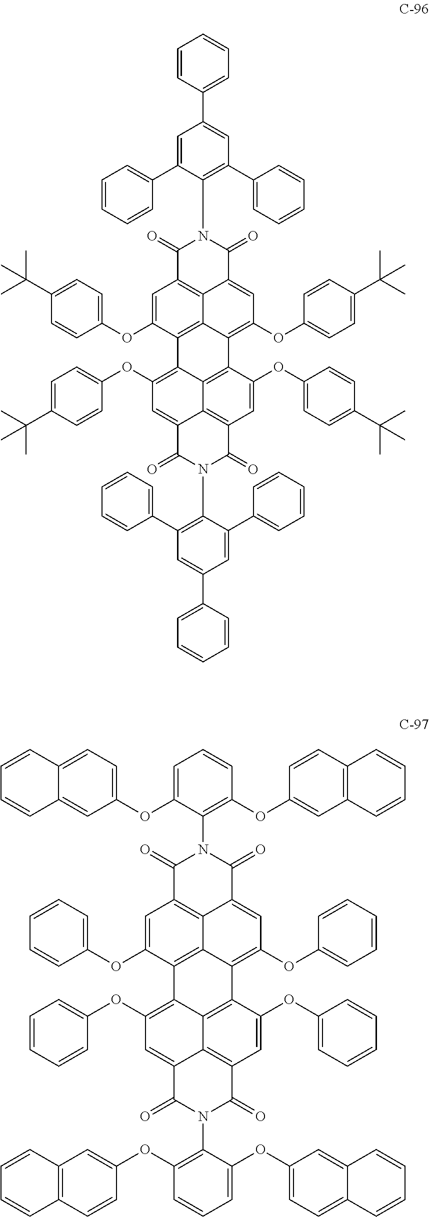

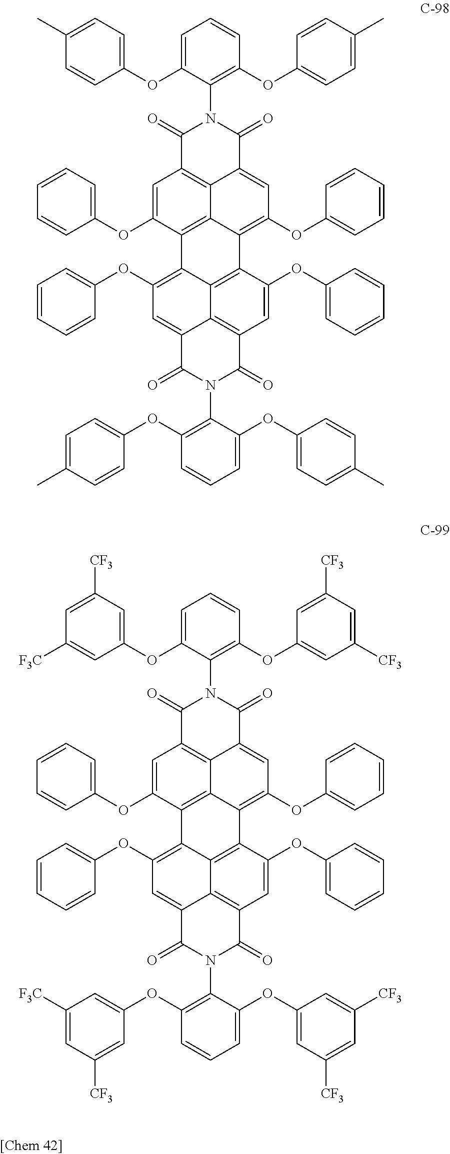

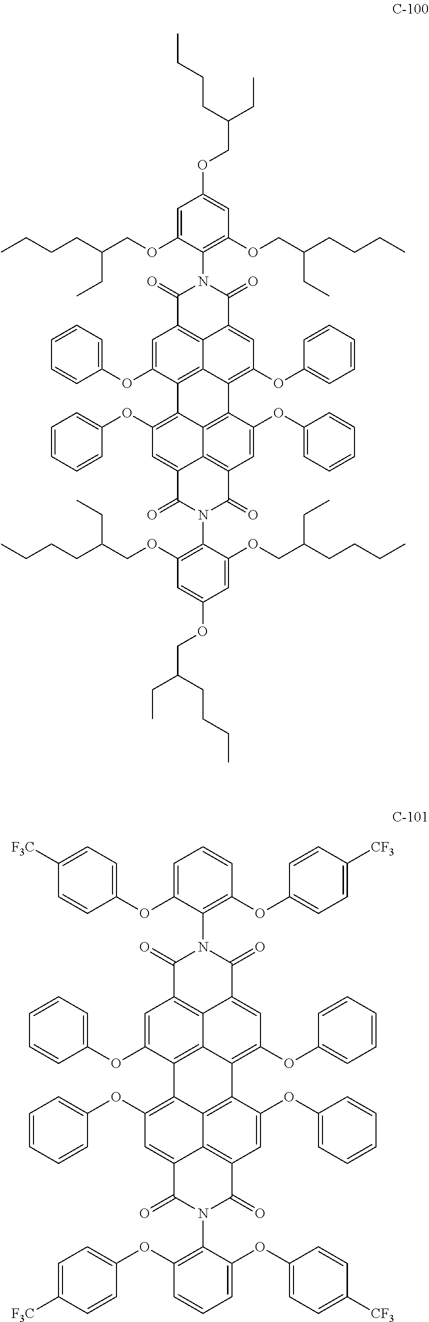

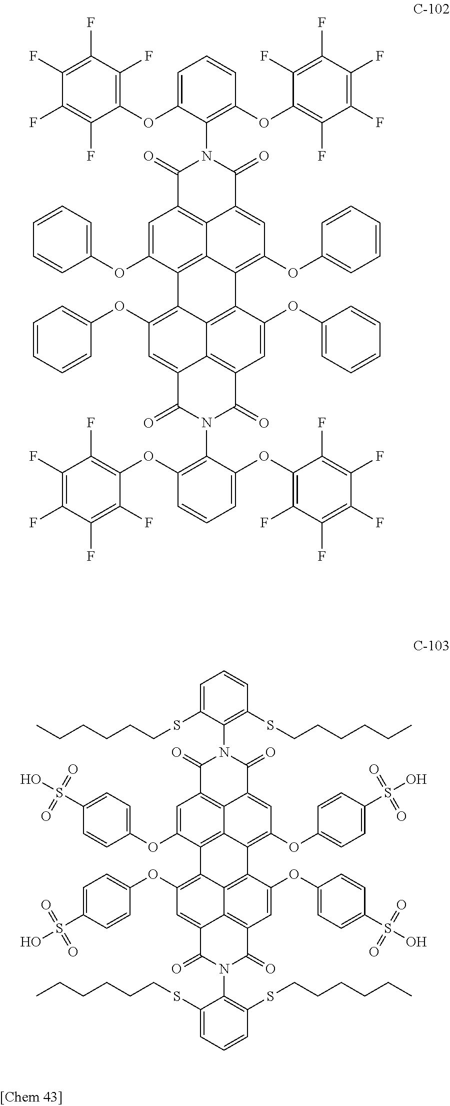

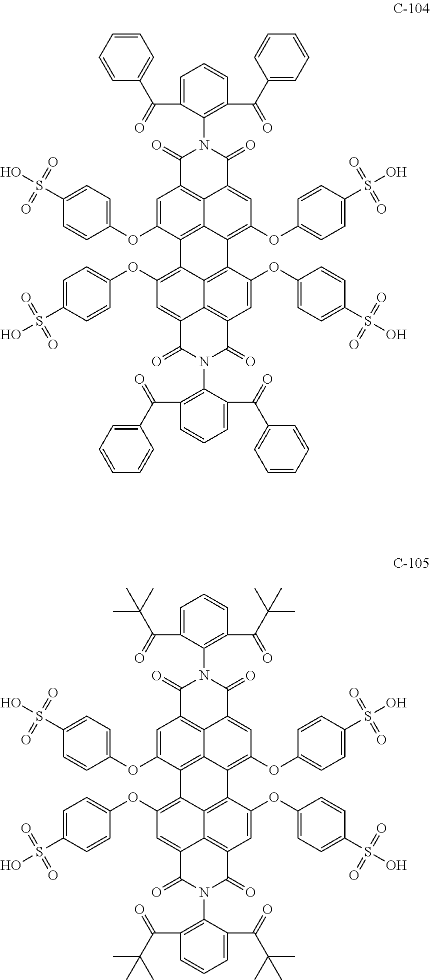

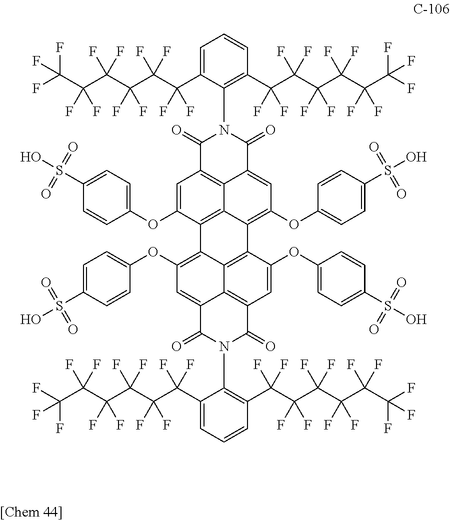

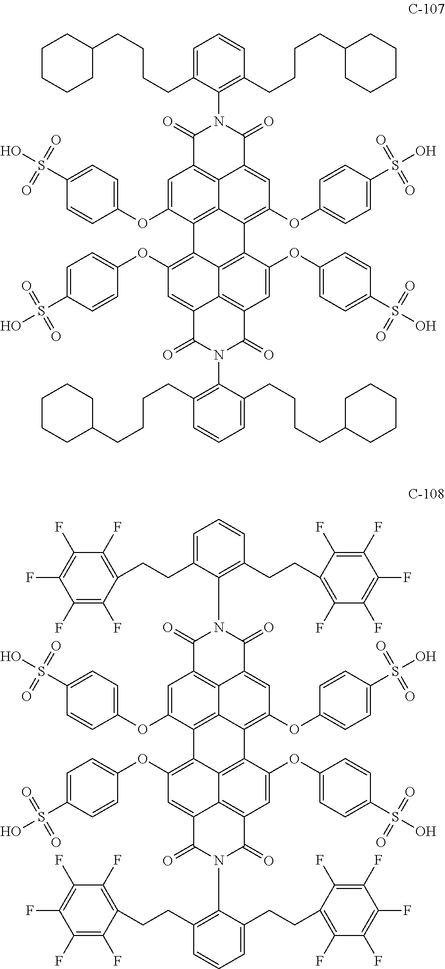

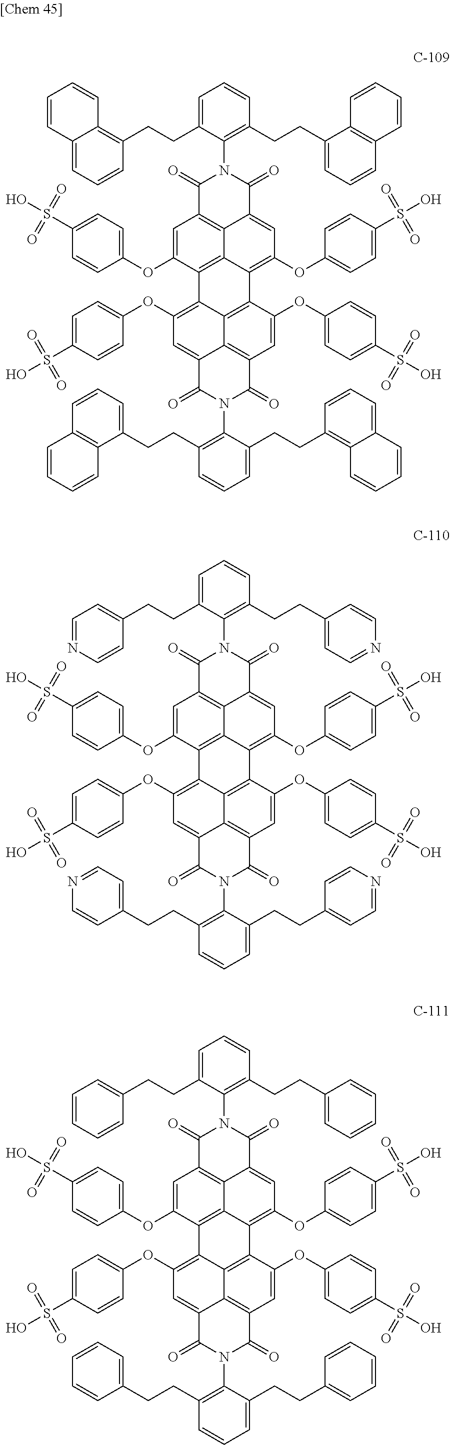

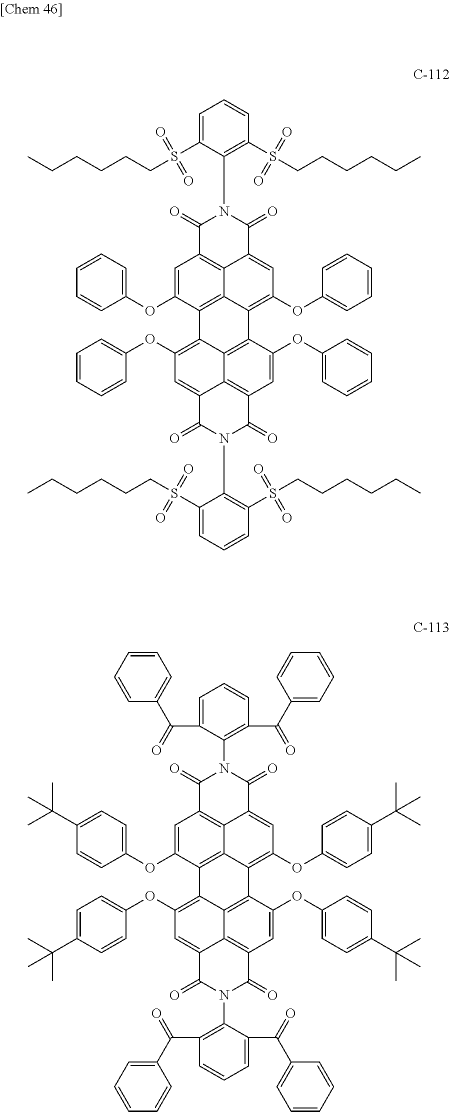

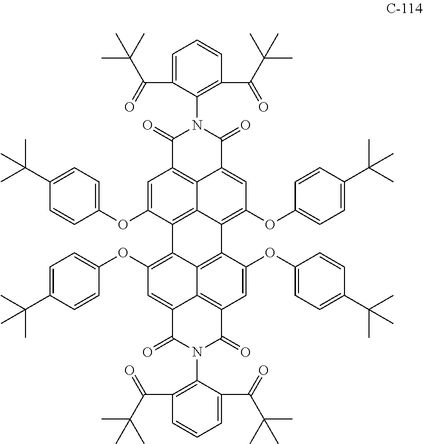

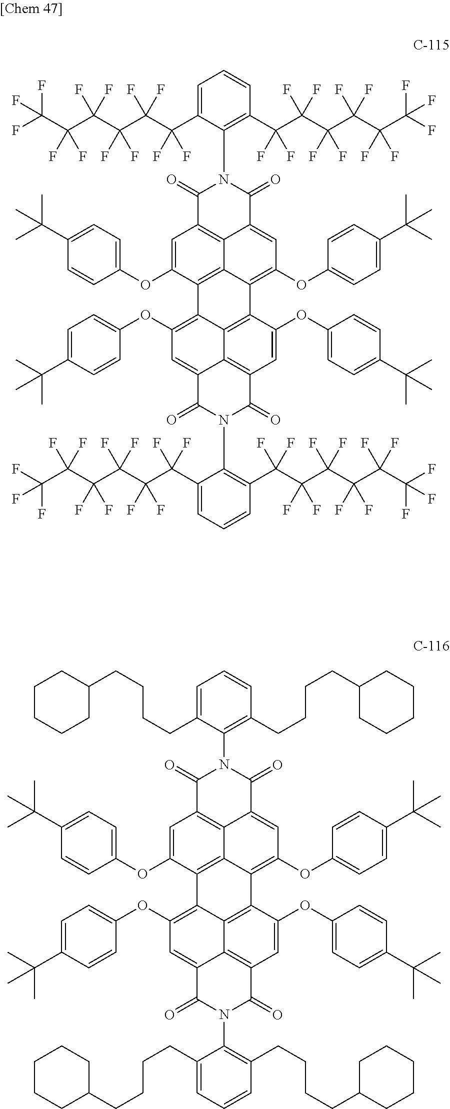

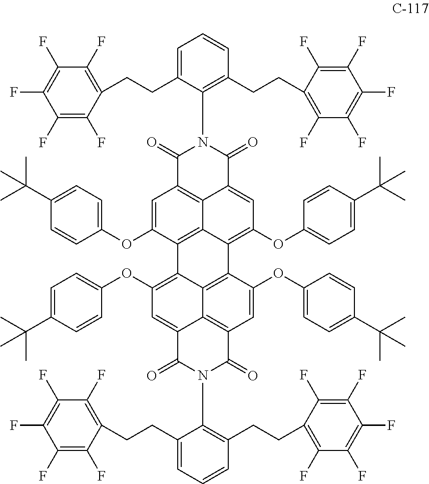

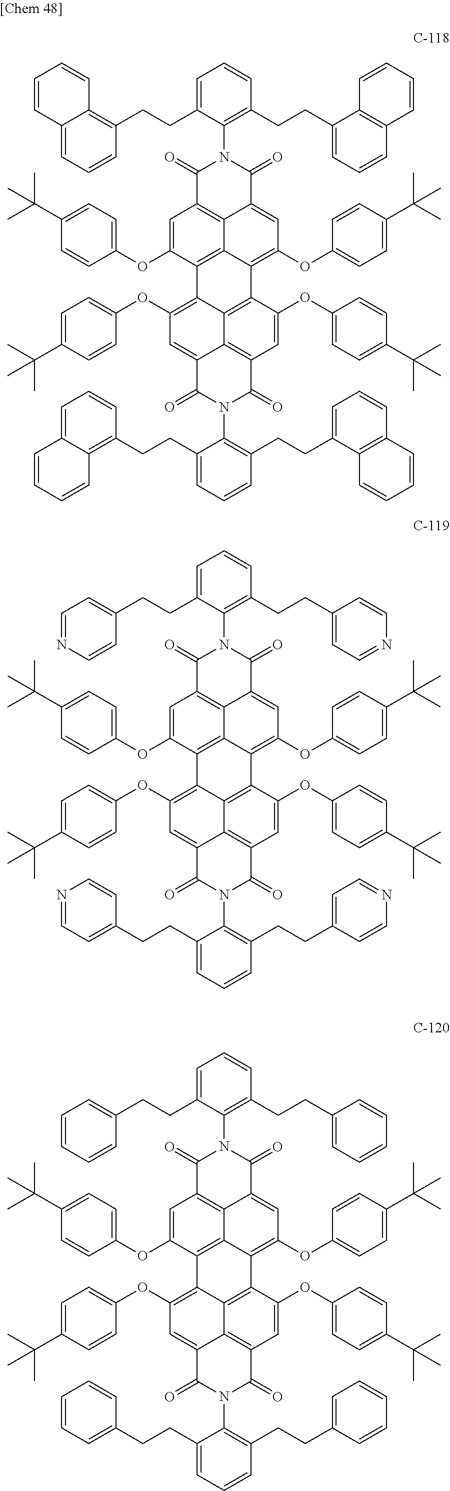

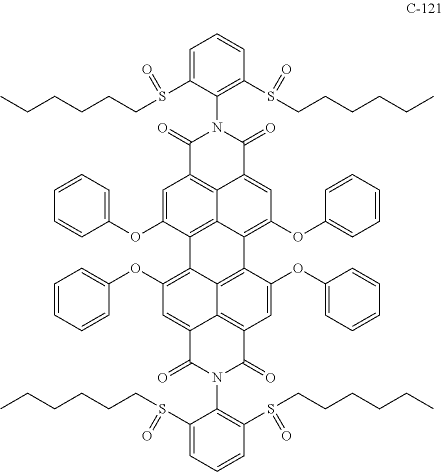

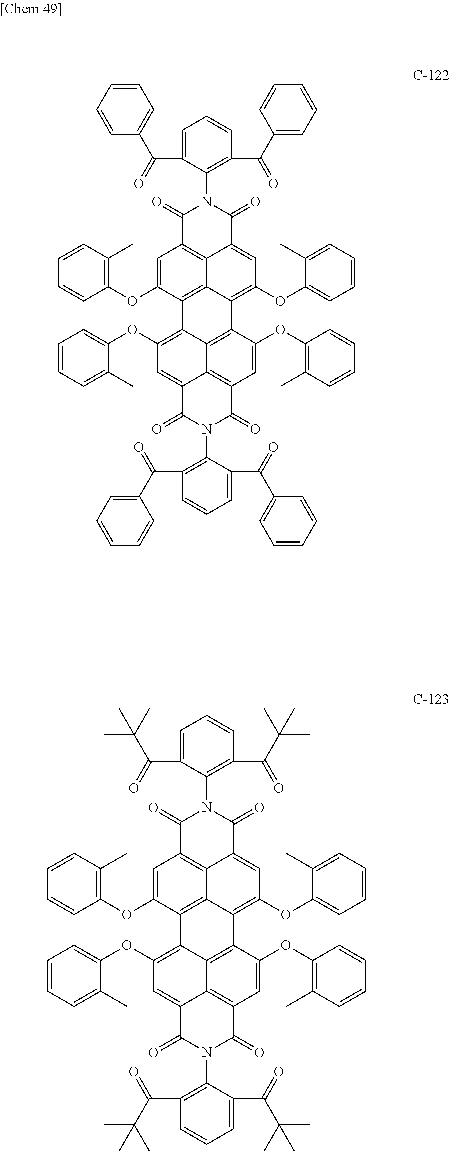

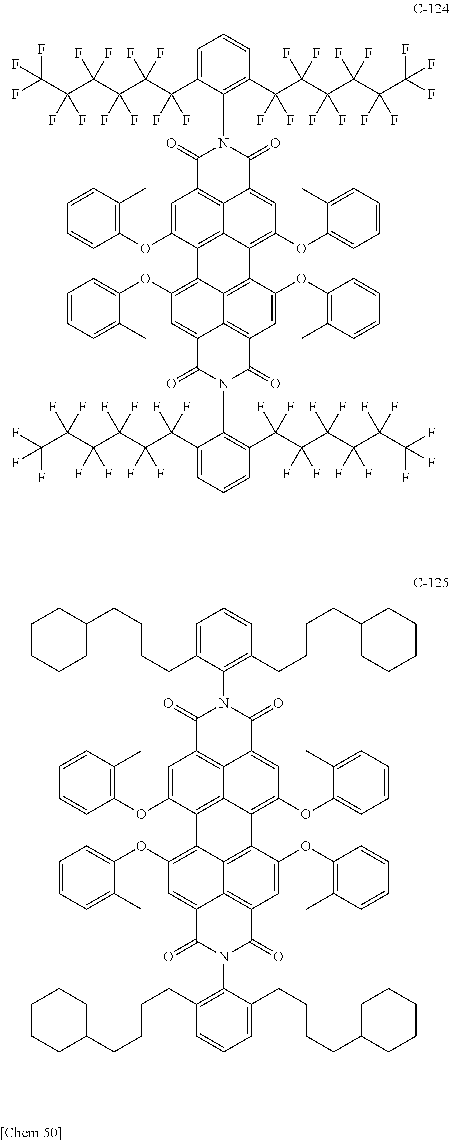

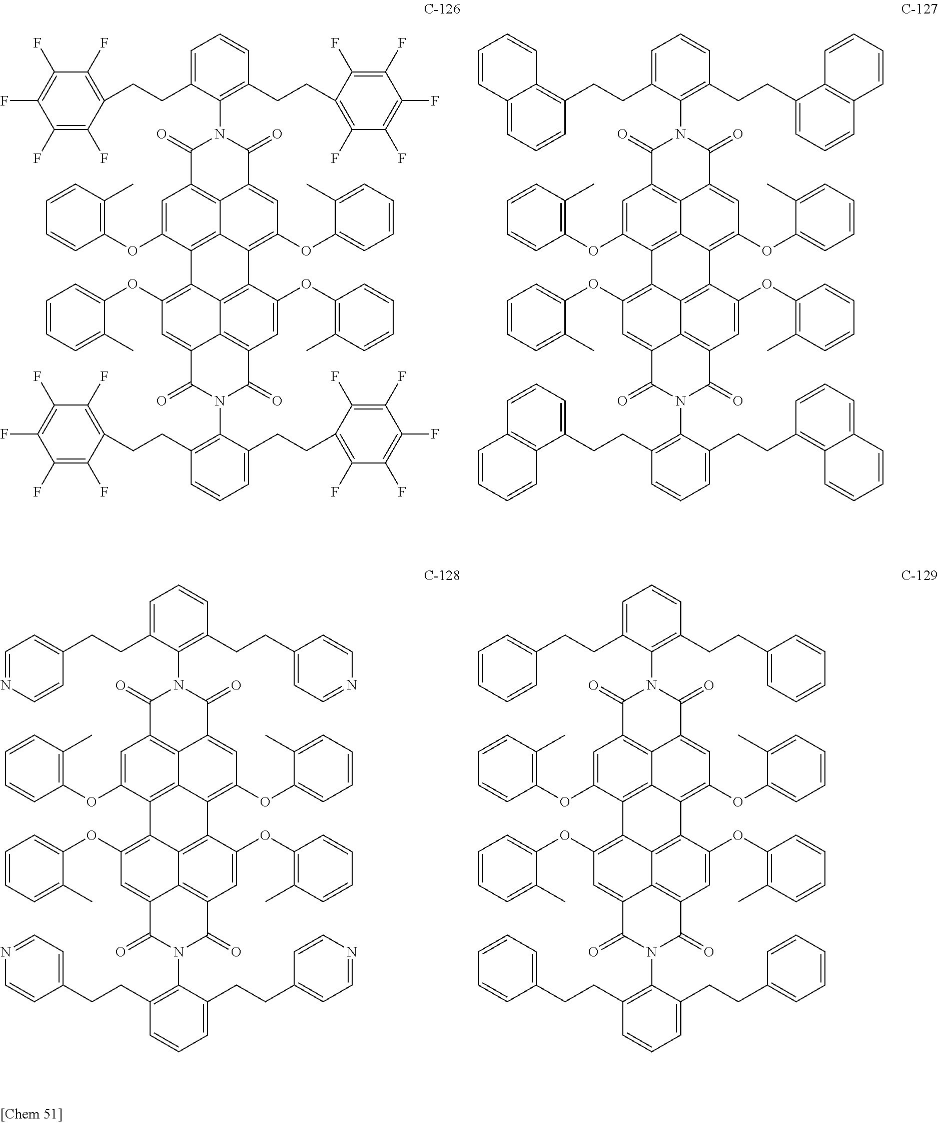

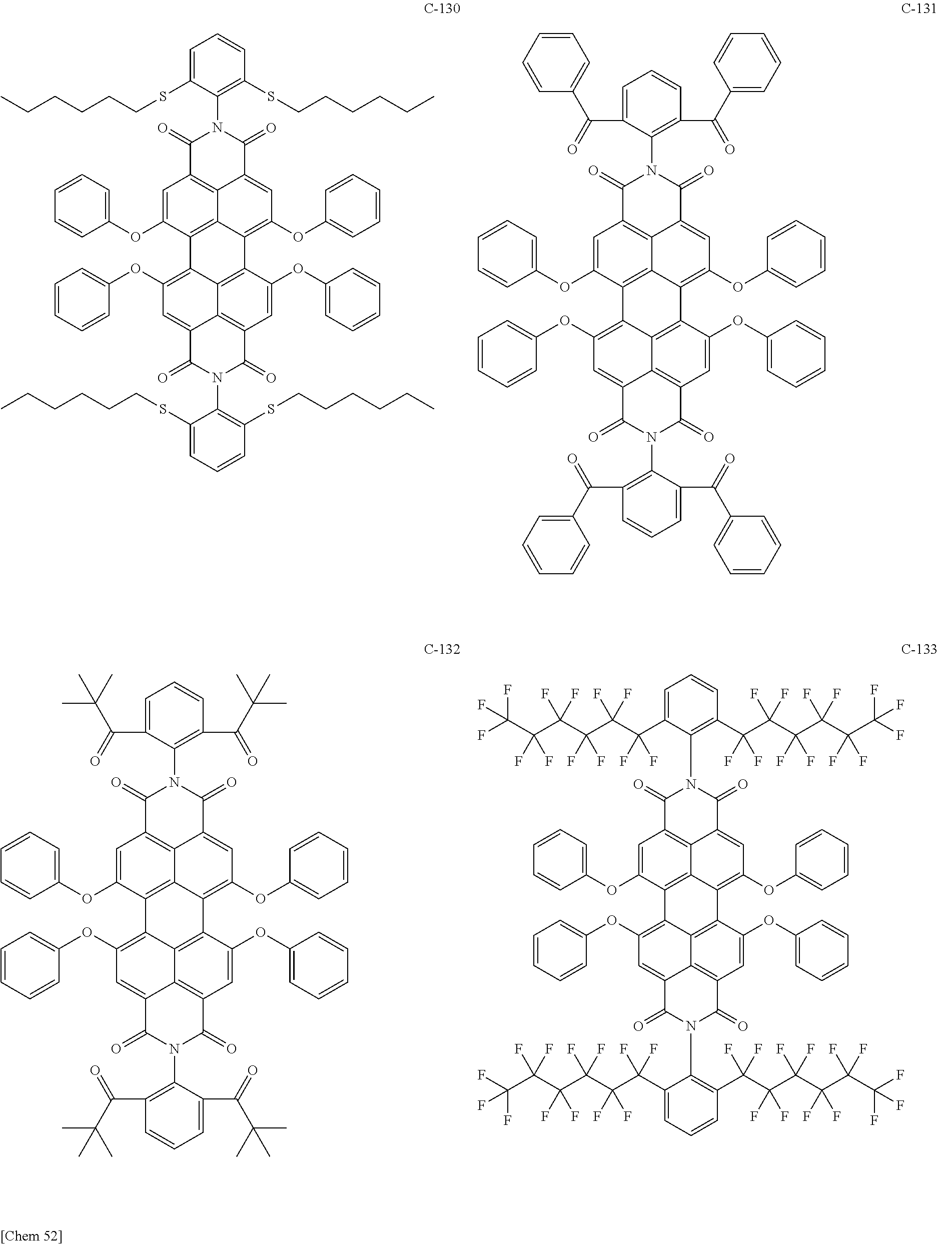

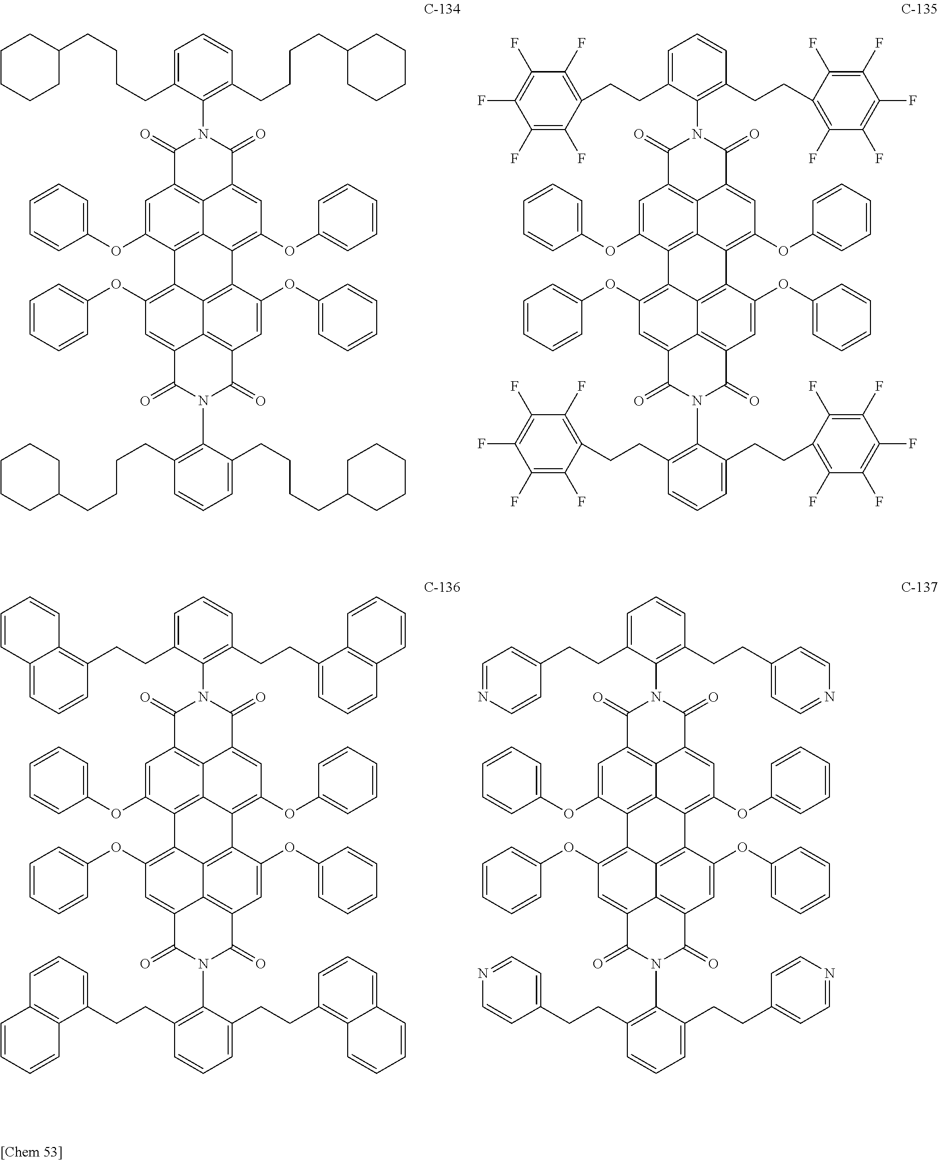

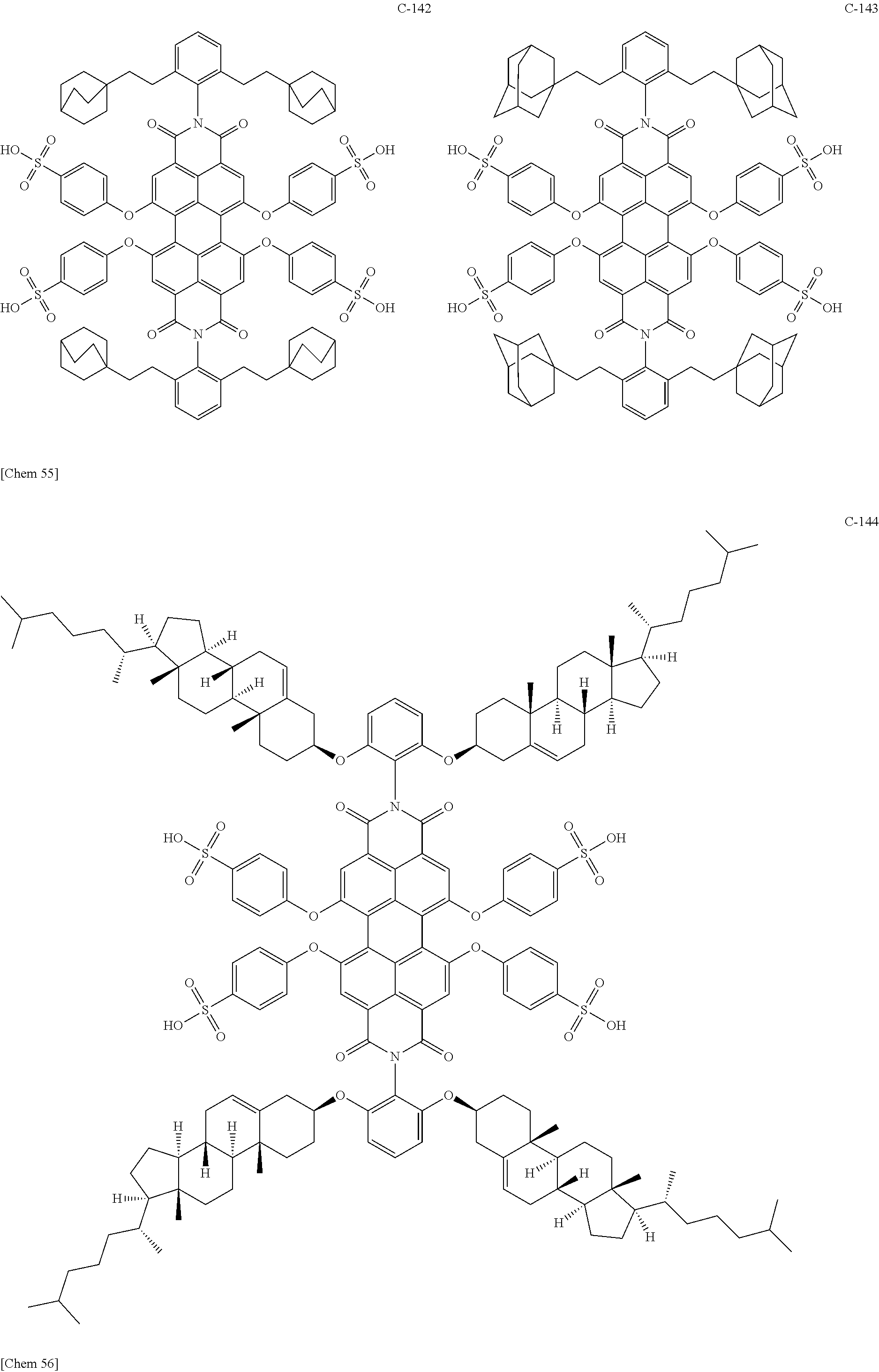

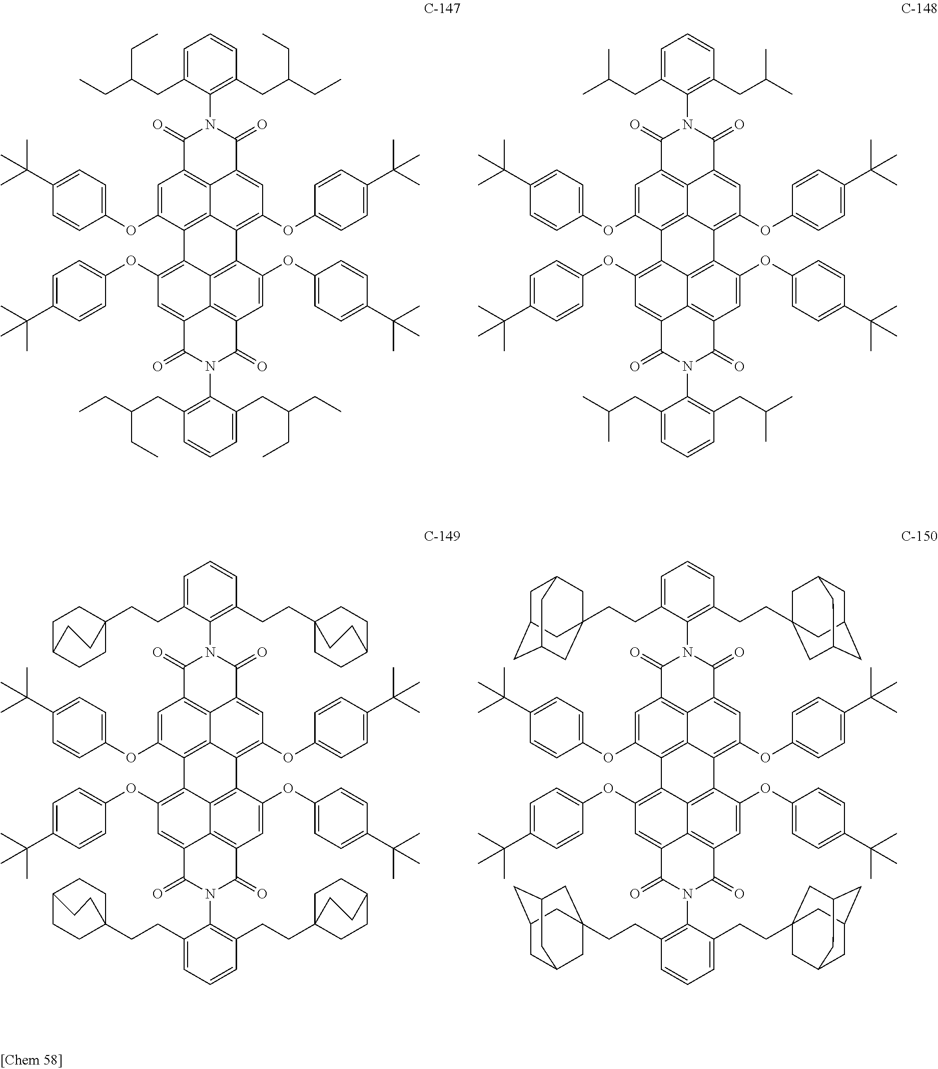

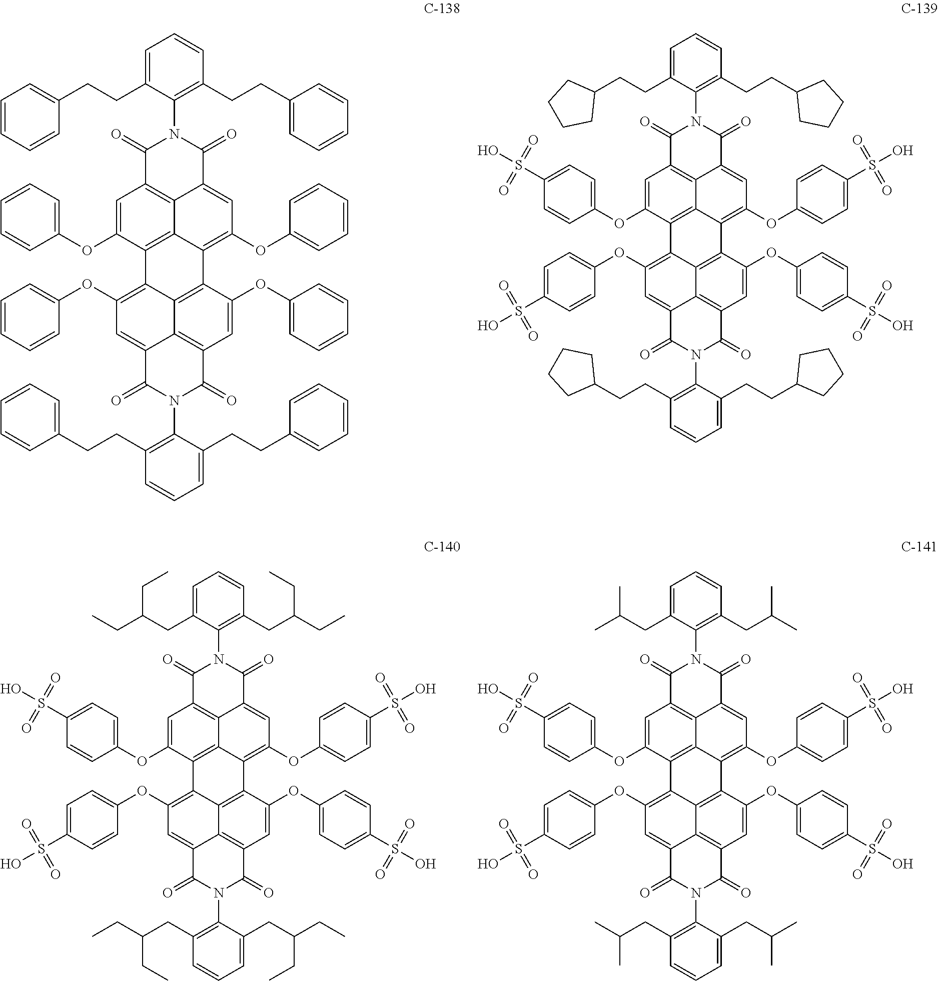

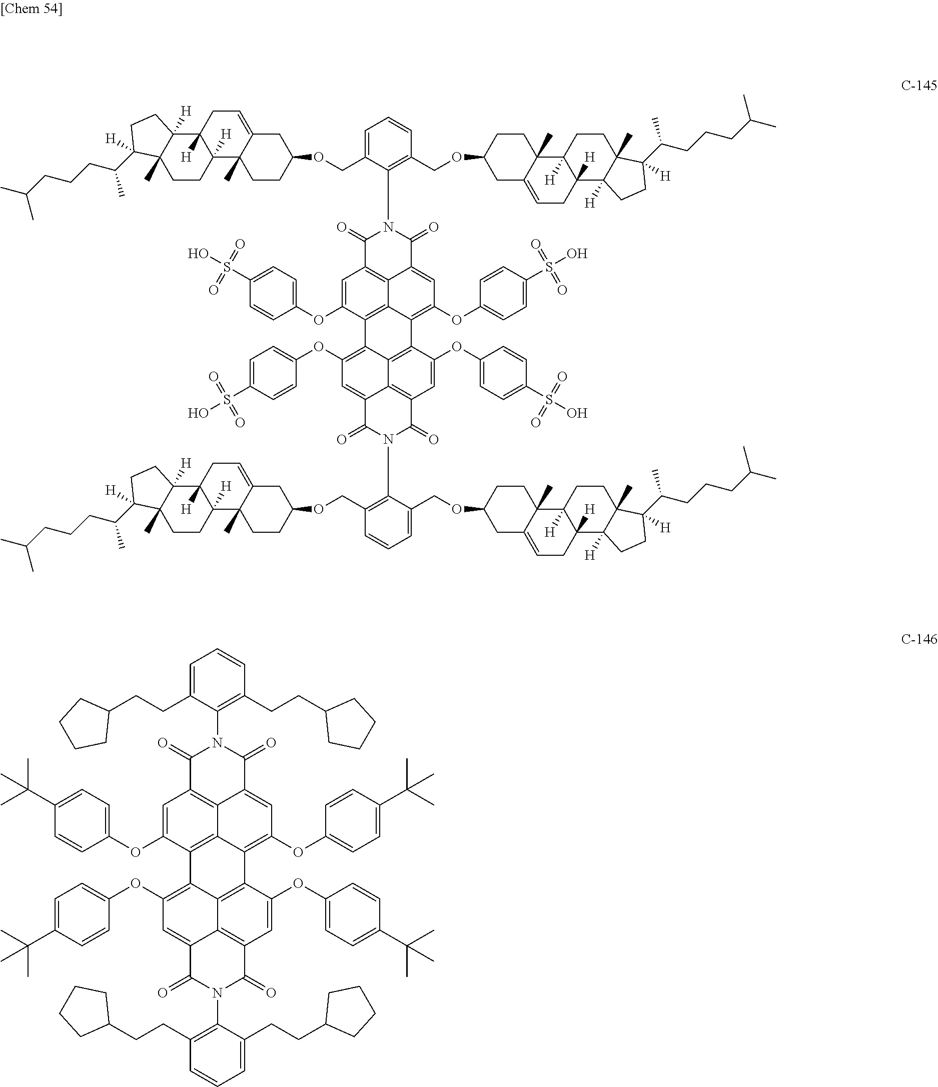

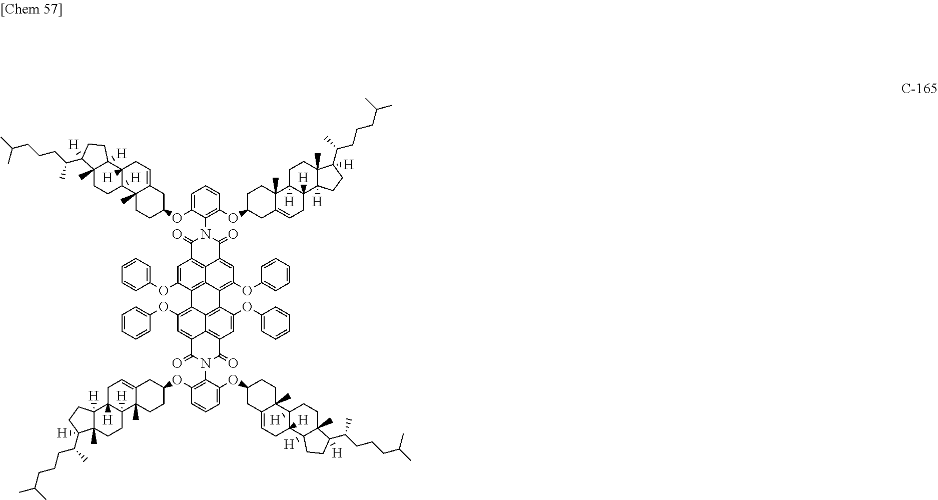

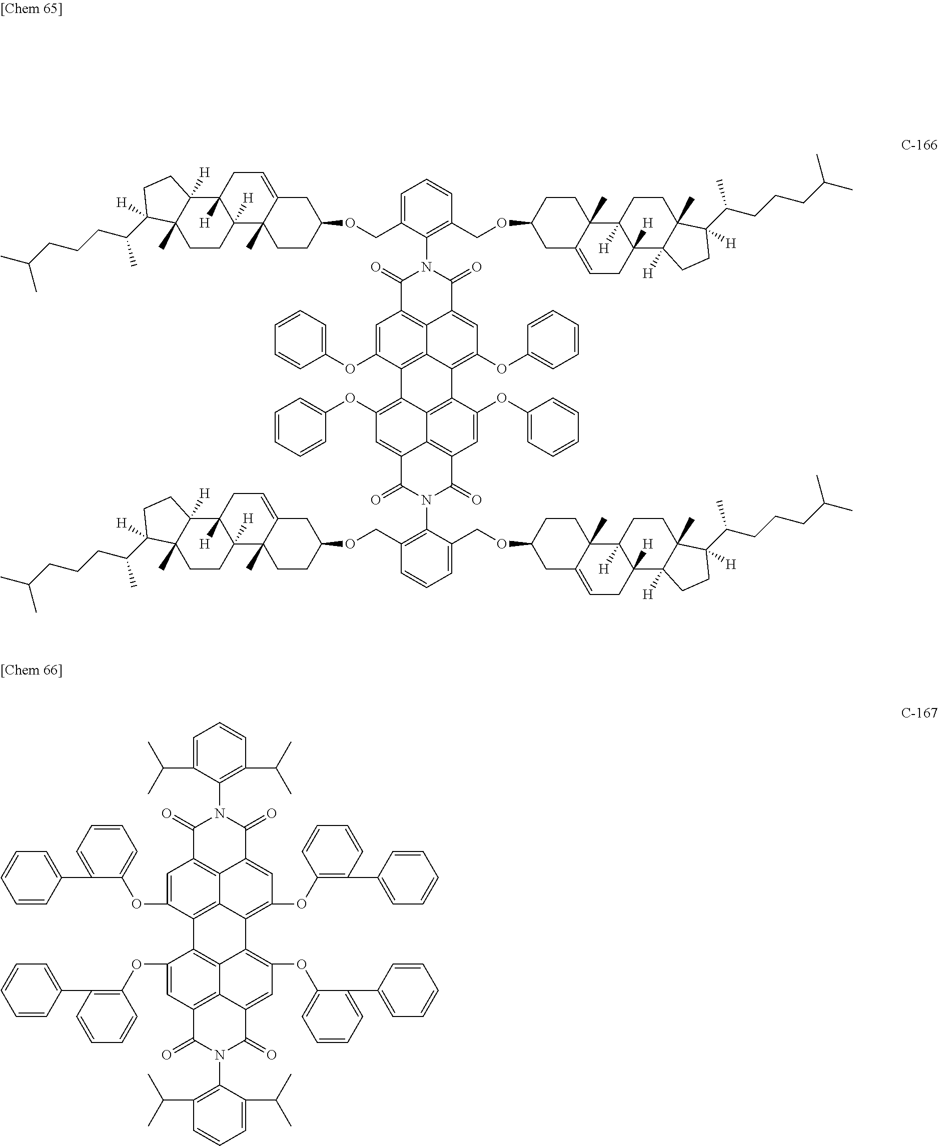

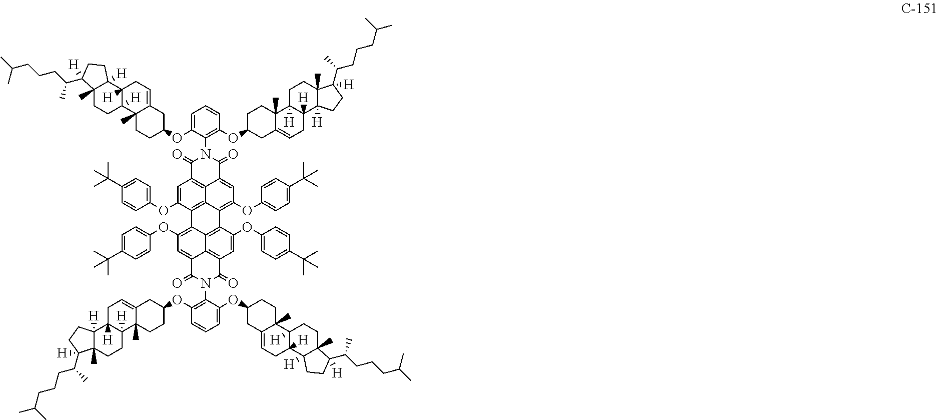

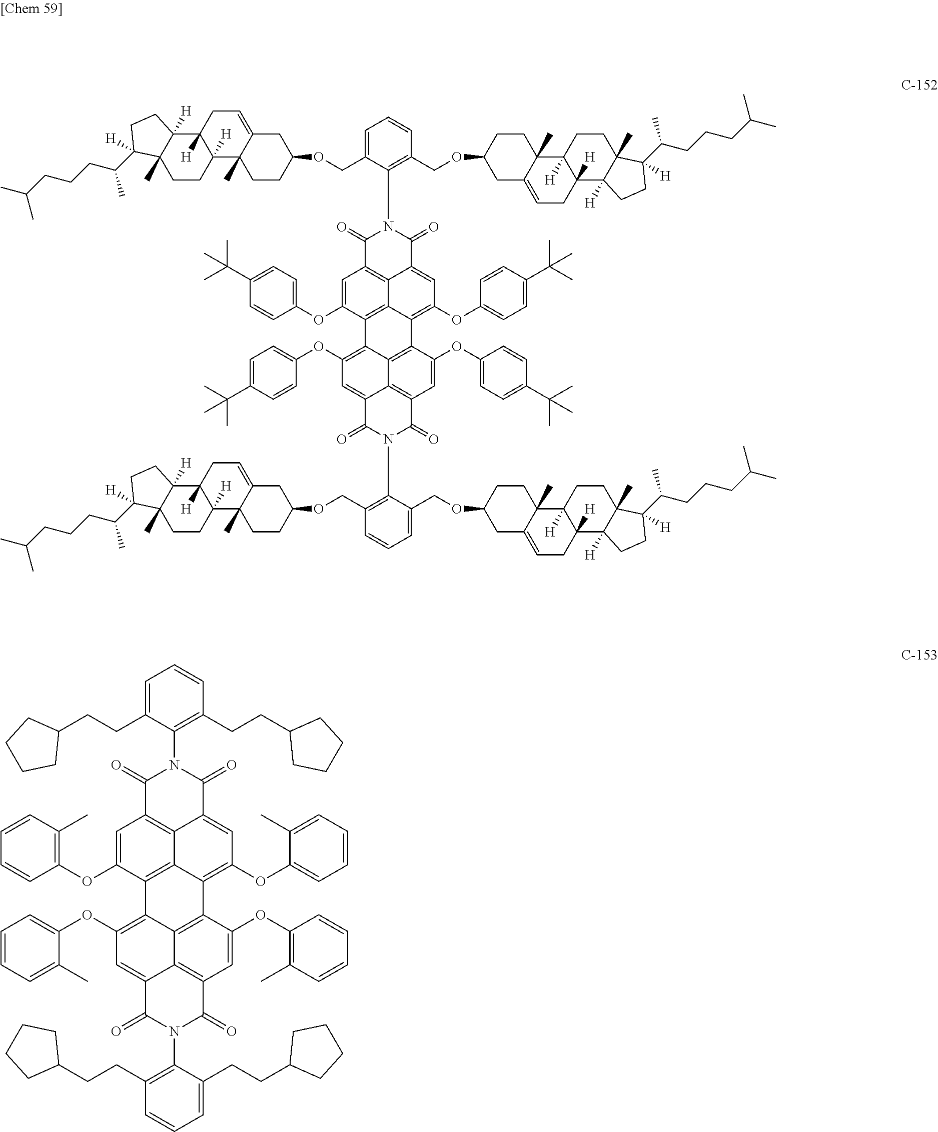

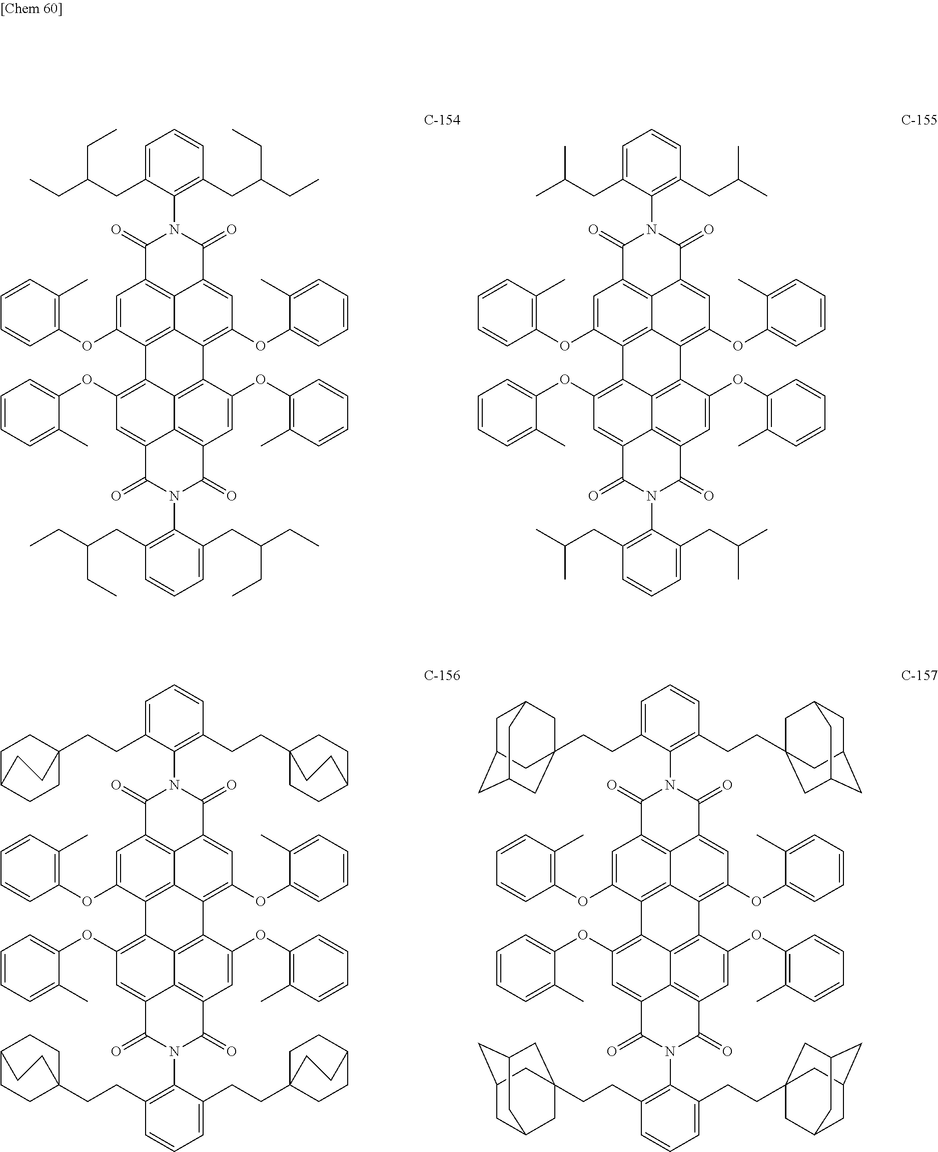

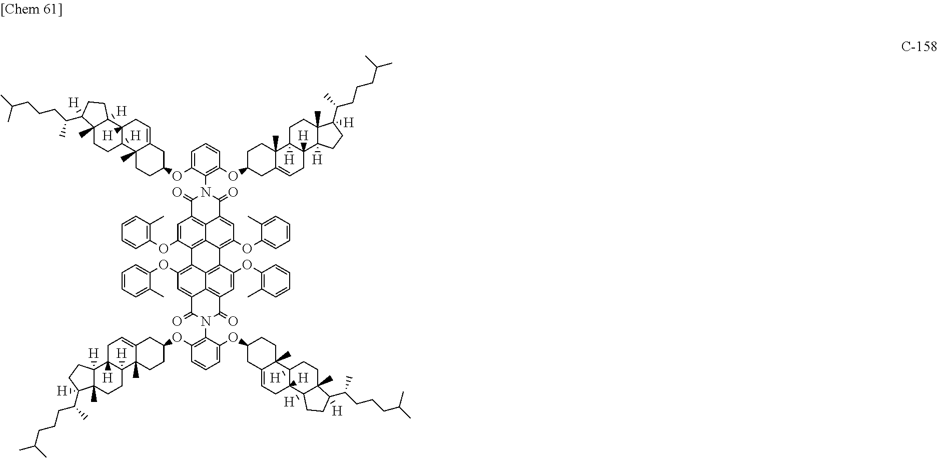

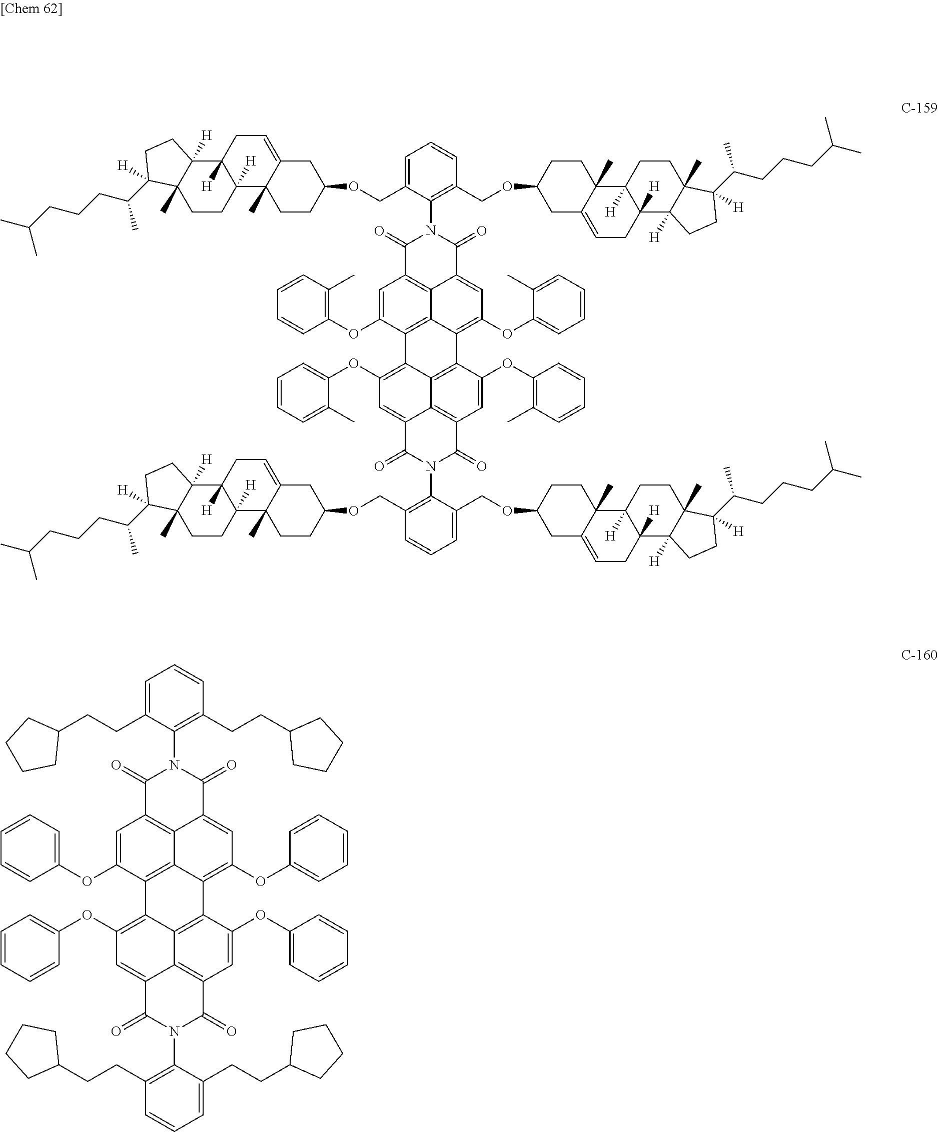

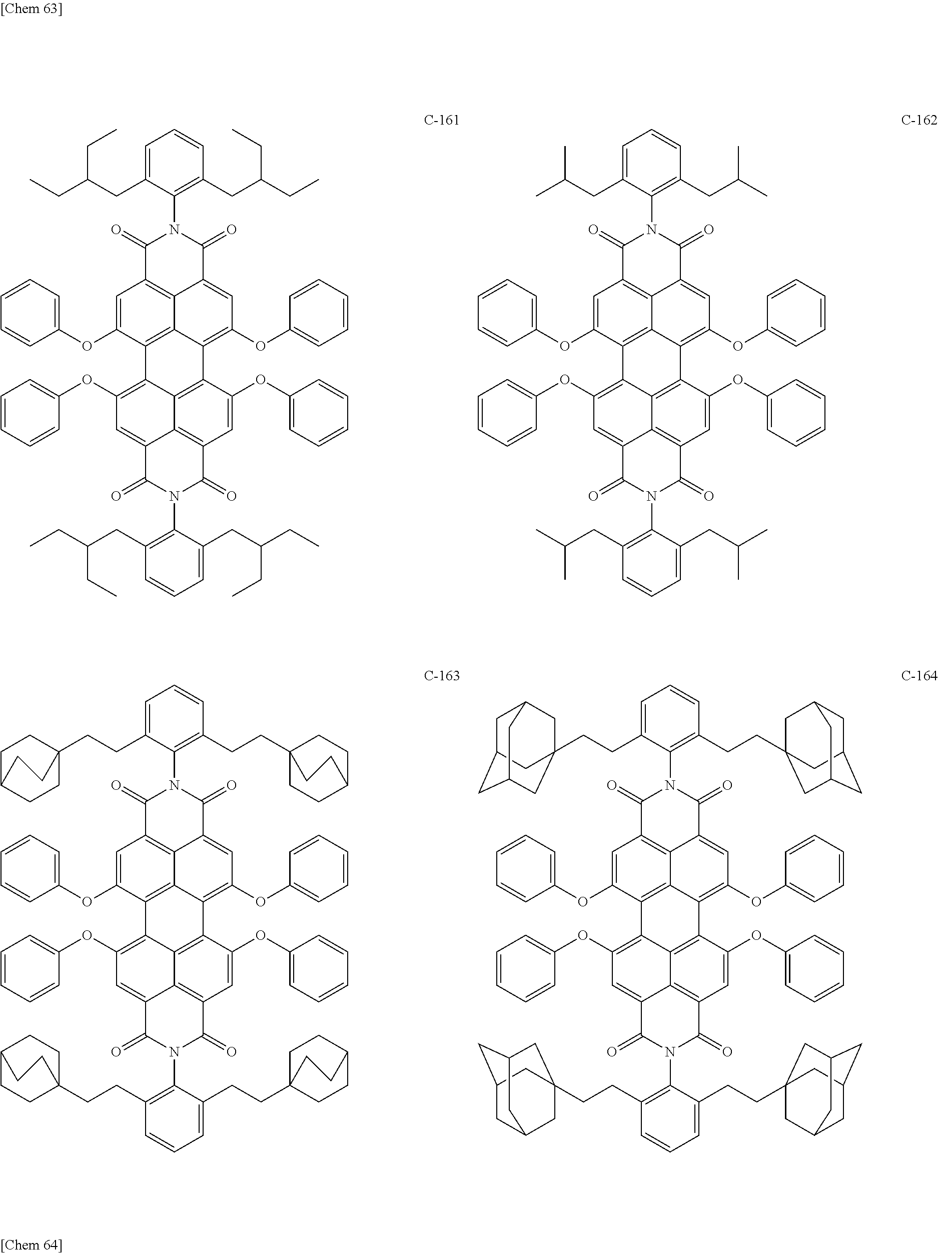

### (Synthesis of Compound (1) and Compound (2))

The compound (1) and compound (2) can be synthesized by known methods, for example, referring to Chem. Eur. J. 2004, 10, 5297-5310. The compound C-53 (compound (1)) and C-45 (compound (2) are synthesized by the following method. Other exemplary compounds can be synthesized in a similar manner. In the synthetic scheme, NMP represents N-methyl-2-pyrrolidone.

### <Synthesis of Exemplified Compound C-53>

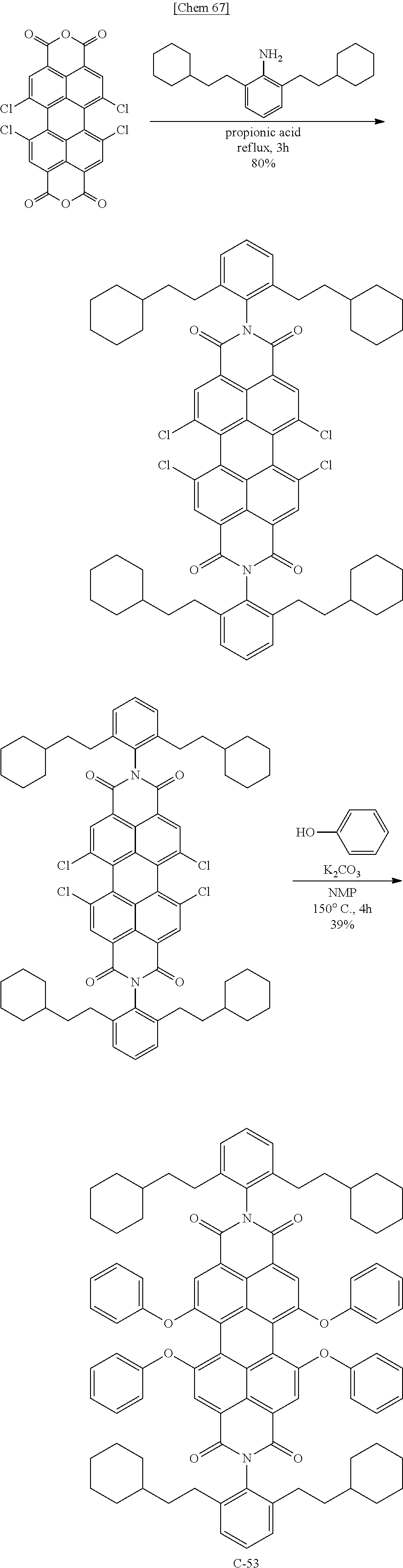

### <Synthesis of Exemplified Compound C-45>

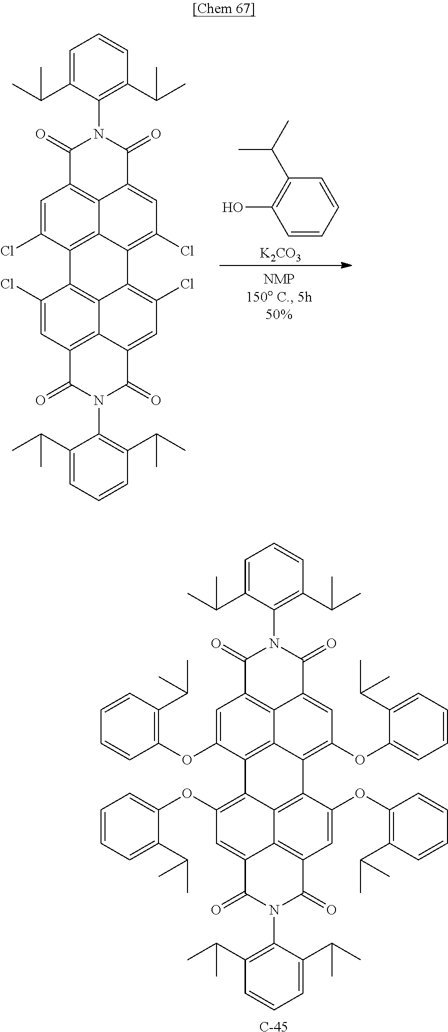

### (Compound (3))

The compound (3) is a compound having a structure represented by the following general formula (3).

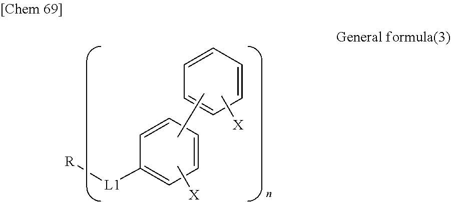

In the general formula (3), R represents a luminescent compound skeleton. Each X independently represents an ionic substituent. L1 represents a single bond, an oxygen-atom, a sulfur-atom, a selenium-atom or a NH group. n represents an integer of 1 or more.

In the compound (3), a luminescent compound skeleton represented by R preferably has any one of the following core compounds.

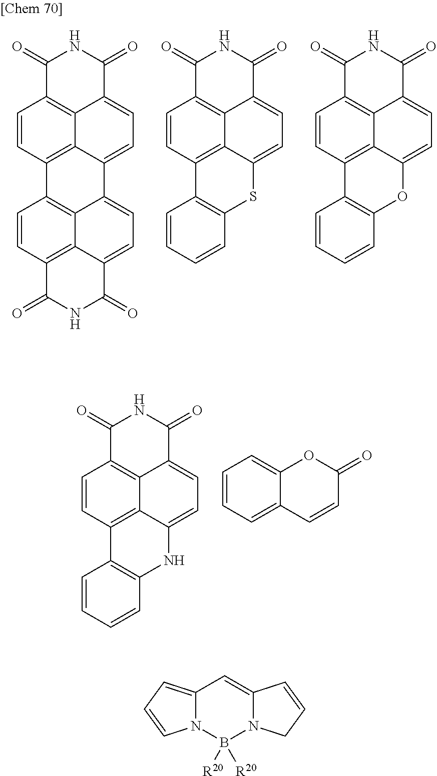

In the above formulae, R²⁰ represents a halogen-atom or a cyano-group.

The compound (3) is a compound in which n of the hydrogen atoms of the core compound are substituted with a substituent (hereinafter, sometimes referred to as a substituent (F3)) surrounded by parentheses in the above-described general formula (3). n is an integer of 1 or more, and is appropriately selected according to the structure of the core compound. n is preferably from 1 to 6, more preferably from 2 to 4. For example, when the core compound is perylene bisimide, n is preferably 2 to 6, and particularly preferably 4. The substituted position of the substituent (F3) in the core compound is not particularly limited, but is preferably a position where the steric hindrance increases. For example, when the core compound is a perylene bisimide, the bay area is preferable.

The substituent (F3) is a substituent in which a biphenyl skeleton and a L1 as a linking group are bonded to each other, and each of the two benzene rings has one ionic substituent X. Of the hydrogen atoms included in the core compound, a hydrogen atom not substituted with a substituent (F3) may be substituted with a substituent other than the substituent (F3).

In compound (3), the ionic substituents represented by X are specifically —OH, —SH, —COOH, —C(═O)H, —S(═O)₂OH, —S(═O)NH₂, —S(═O)₂NH₂, —P(═O)(OH)₃, —P(═O)R(OH)₂, —P(═O)R²(OH), —P(OH)₃, —P(═O)(NH₂)₃, —P(═O)R(NH₂)₂, —P(═O)R²(NH₂), —P(NH₂)₃, —O(C═O)OH, —NH₂, —NHR, —NHCONH₂, —NHCONHR, —NHCOOH, —Si(OH)₃, —Si(R)(OH)₂, —Si(R)₂OH, —Ge(OH)₃, —Ge(R)(OH)₂, —Ge(R)₂OH, —Ti(OH)₃, —Ti(R)(OH)₂, —Ti(R)₂OH, —Si(NH₂)₃, —Si(R)(NH₂)₂, —B(OH)₂, —O—B(OH)₂, —B(NH₂)₂, —NHB(OH)₂, polyethylene glycol group, etc. Each R independently represents hydrogen or an alkyl group having 1 to 20 carbon atoms. Other examples of the ionic substituent include a NHS group and a maleimide group.

The ionic substituent is preferably any of a sulfo group, a phosphoric acid group, a sulfonic acid ester group, a phosphoric acid ester group, or an ammonium group, a carboxy group, a phosphonium group, or a salt thereof. Among these, a sulfo group, a phosphoric acid group, a sulfonic acid ester group, a phosphoric acid ester group, an ammonium group, or a salt thereof is more preferable, and particularly, a sulfo group or a salt thereof is more preferable. Specific examples thereof include —SO₃H, —SO₃Na, —OSO₃H, —OSO₃Na, —SO₃NH₄, —PO₄H₂, —PO₄Na, —OPO₃H₂, —OPO₃Na₂, —NMe₃OH, —NMe₃Cl.

In the compound (3), L represents a single bond, an oxygen atom, a sulfur atom, a selenium atom, or a NH group, and is preferably an oxygen atom.

It is preferable that the compound (3) has a structure represented by the following general formula (4) from the viewpoint of excellent density quenching suppressing effect.

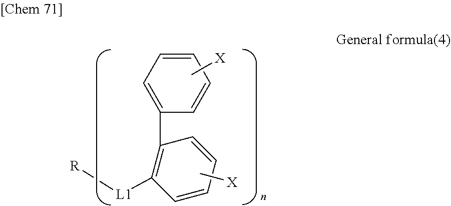

In the general formula (4), R represents a luminescent compound skeleton. X represents an ionic substituent. L1 represents a single bond, an oxygen-atom, a sulfur-atom, a selenium-atom or a NH group.

In the general formula (4), R, X, and L1 have the same meanings as R, X, and L1 in the general formula (3).

Further, it is preferable that the compound (4) has a structure represented by the following general formula (9) from the viewpoint of excellent density quenching suppressing effect.

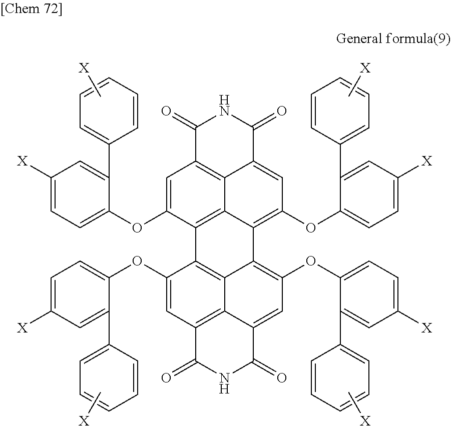

In the general formula (9), X represents a sulfo group or a salt thereof. H in NH may be substituted with a substituent.

In the compound (9), examples of the substituent when H in NH is substituted with a substituent include a substituted or unsubstituted alkyl group, aryl group or heteroaryl group. Specific examples thereof may be the same as those of R² in the general formula (2).

Specific examples of the compound represented by a luminescent compound (3) are set forth below, but the present invention is not limited thereto.

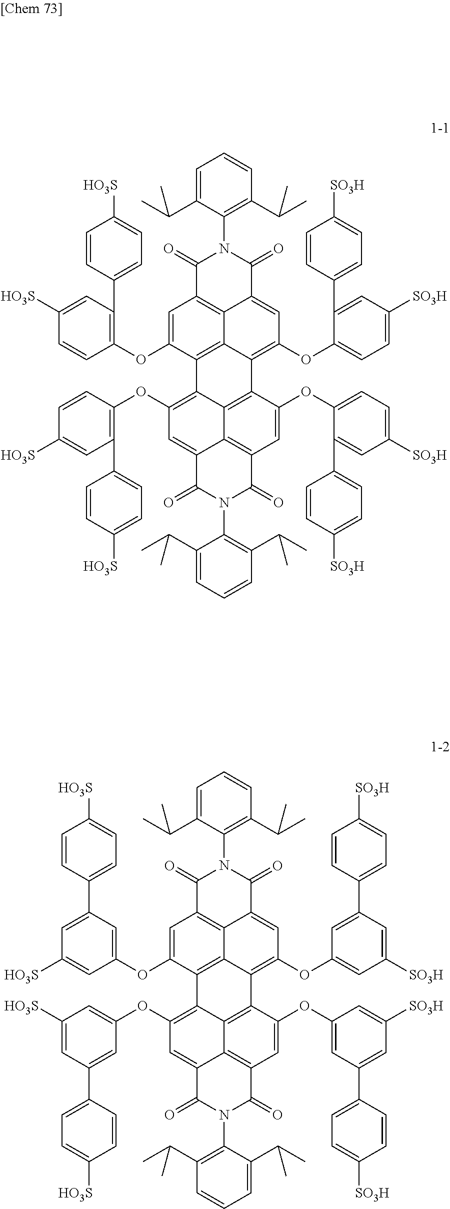

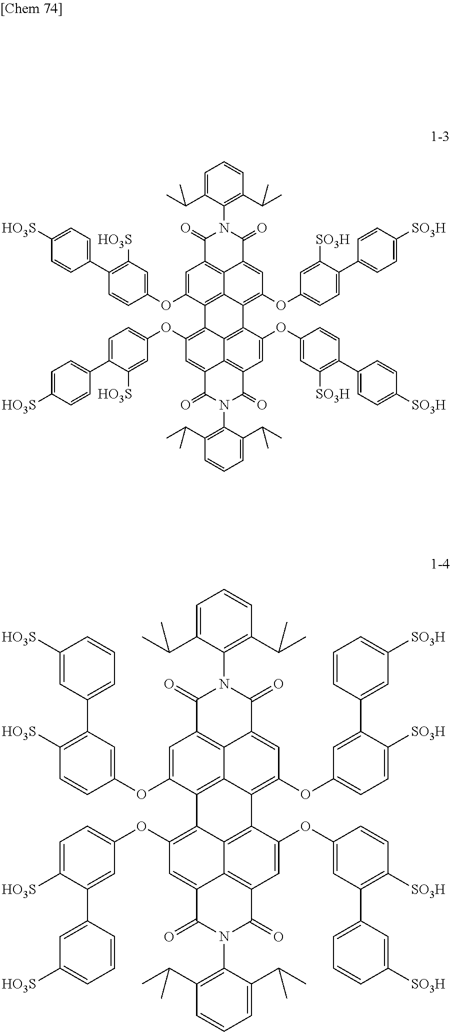

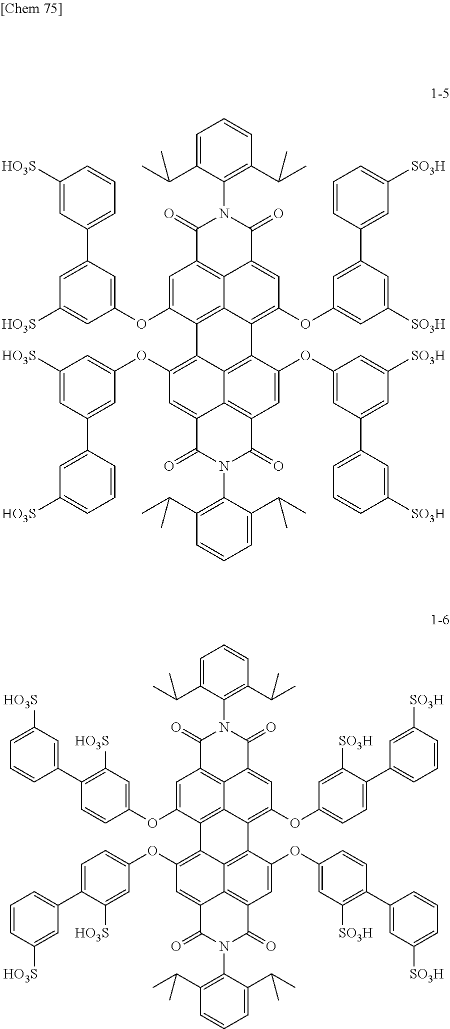

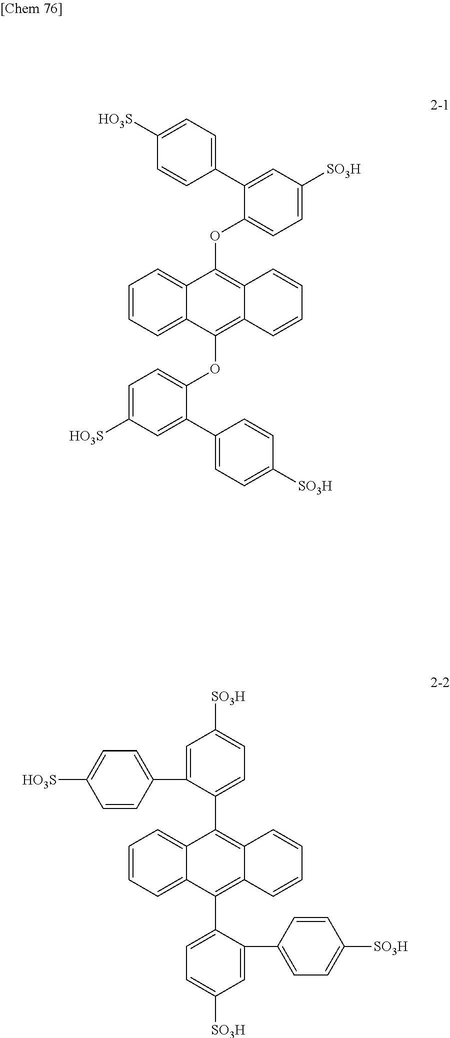 [SMILES: O=S(=O)(O)c1ccc(-c2cc(S(=O)(=O)O)ccc2Oc2c3ccccc3c(Oc3ccc(S(=O)(=O)O)cc3-c3ccc(S(=O)(=O)O)cc3)c3ccccc23)cc1 ; O=S(=O)(O)c1ccc(-c2cc(S(=O)(=O)O)ccc2-c2c3ccccc3c(-c3ccc(S(=O)(=O)O)cc3-c3ccc(S(=O)(=O)O)cc3)c3ccccc23)cc1]

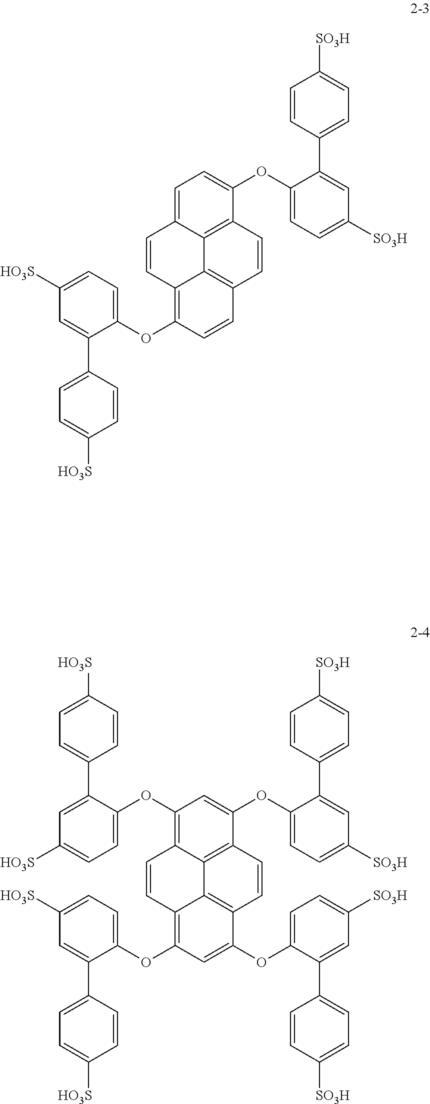 [SMILES: O=S(=O)(O)c1ccc(-c2cc(S(=O)(=O)O)ccc2Oc2ccc3ccc4c(Oc5ccc(S(=O)(=O)O)cc5-c5ccc(S(=O)(=O)O)cc5)ccc5ccc2c3c54)cc1 ; O=S(=O)(O)c1ccc(-c2cc(S(=O)(=O)O)ccc2Oc2cc(Oc3ccc(S(=O)(=O)O)cc3-c3ccc(S(=O)(=O)O)cc3)c3ccc4c(Oc5ccc(S(=O)(=O)O)cc5-c5ccc(S(=O)(=O)O)cc5)cc(Oc5ccc(S(=O)(=O)O)cc5-c5ccc(S(=O)(=O)O)cc5)c5ccc2c3c54)cc1]

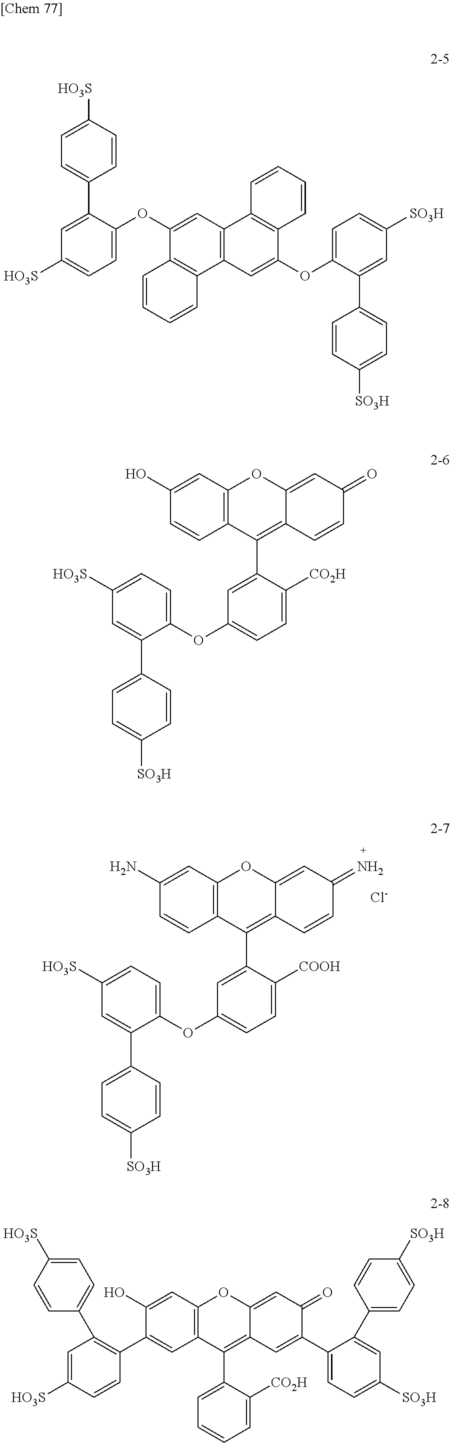 [SMILES: O=S(=O)(O)c1ccc(-c2cc(S(=O)(=O)O)ccc2Oc2cc3c4ccccc4c(Oc4ccc(S(=O)(=O)O)cc4-c4ccc(S(=O)(=O)O)cc4)cc3c3ccccc23)cc1 ; O=C(O)c1ccc(Oc2ccc(S(=O)(=O)O)cc2-c2ccc(S(=O)(=O)O)cc2)cc1-c1c2ccc(=O)cc-2oc2cc(O)ccc12 ; N=c1ccc2c(-c3cc(Oc4ccc(S(=O)(=O)O)cc4-c4ccc(S(=O)(=O)O)cc4)ccc3C(=O)O)c3ccc(N)cc3oc-2c1 ; [Cl-] ; O=C(O)c1ccccc1-c1c2cc(-c3ccc(S(=O)(=O)O)cc3-c3ccc(S(=O)(=O)O)cc3)c(=O)cc-2oc2cc(O)c(-c3ccc(S(=O)(=O)O)cc3-c3ccc(S(=O)(=O)O)cc3)cc12]

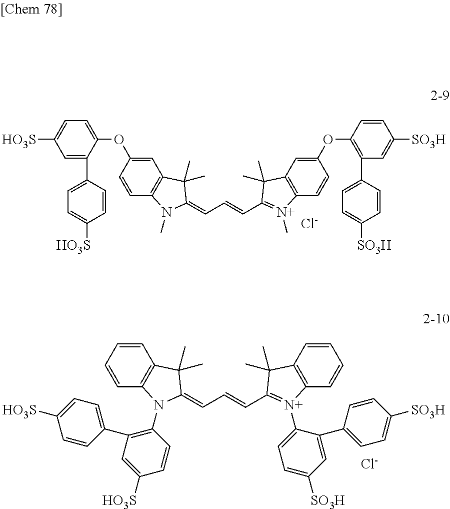 [SMILES: CN1/C(=C/C=C/C2=[N+](C)c3ccc(Oc4ccc(S(=O)(=O)O)cc4-c4ccc(S(=O)(=O)O)cc4)cc3C2(C)C)C(C)(C)c2cc(Oc3ccc(S(=O)(=O)O)cc3-c3ccc(S(=O)(=O)O)cc3)ccc21 ; [Cl-] ; CC1(C)C(/C=C/C=C2/N(c3ccc(S(=O)(=O)O)cc3-c3ccc(S(=O)(=O)O)cc3)c3ccccc3C2(C)C)=[N+](c2ccc(S(=O)(=O)O)cc2-c2ccc(S(=O)(=O)O)cc2)c2ccccc21 ; [Cl-]]

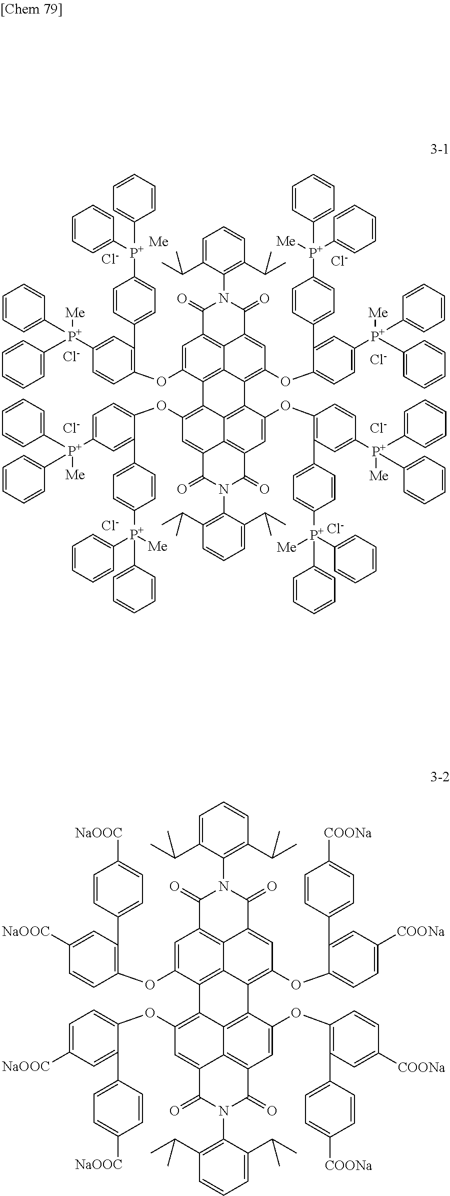

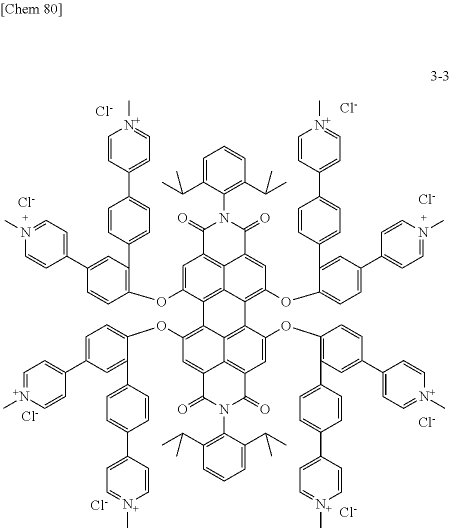

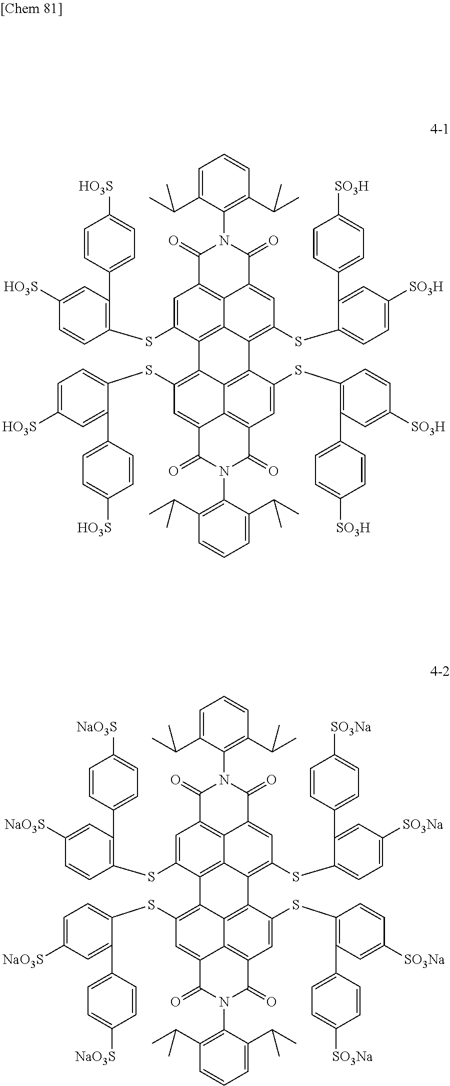

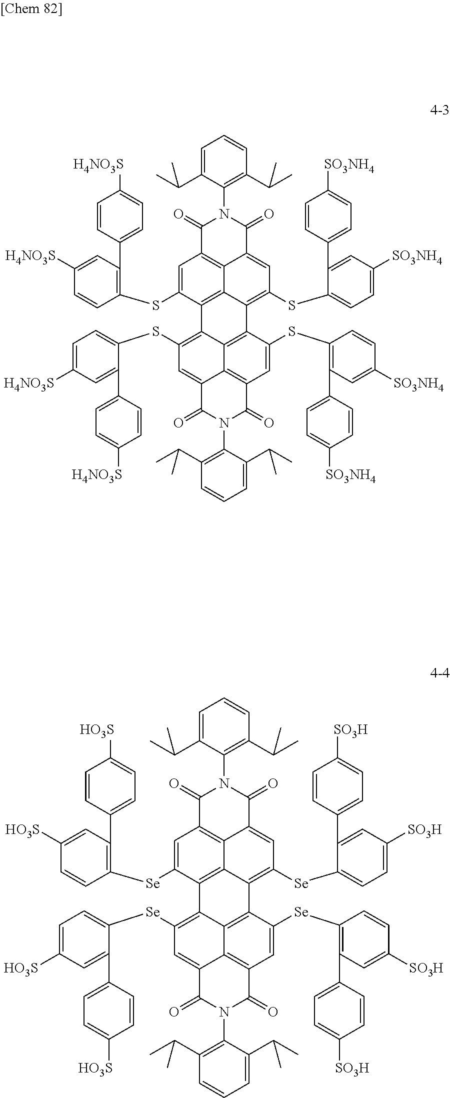

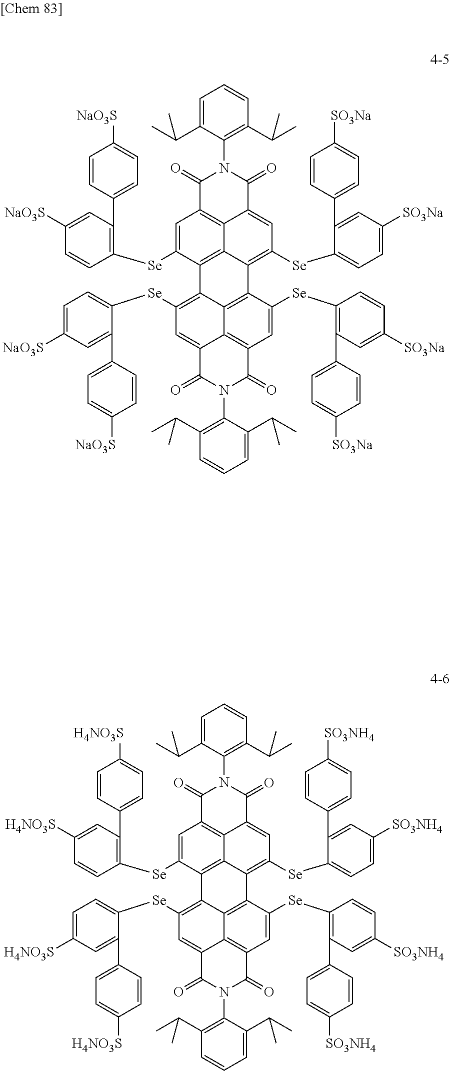

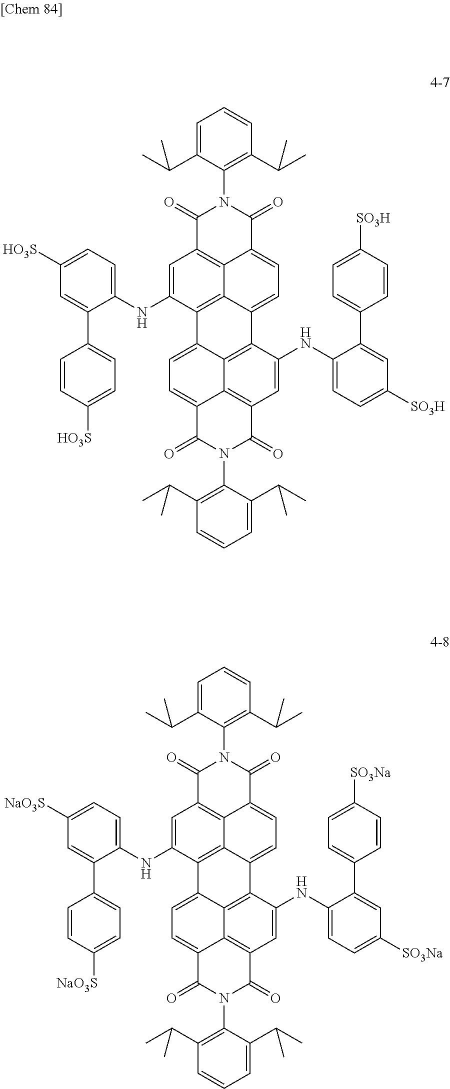

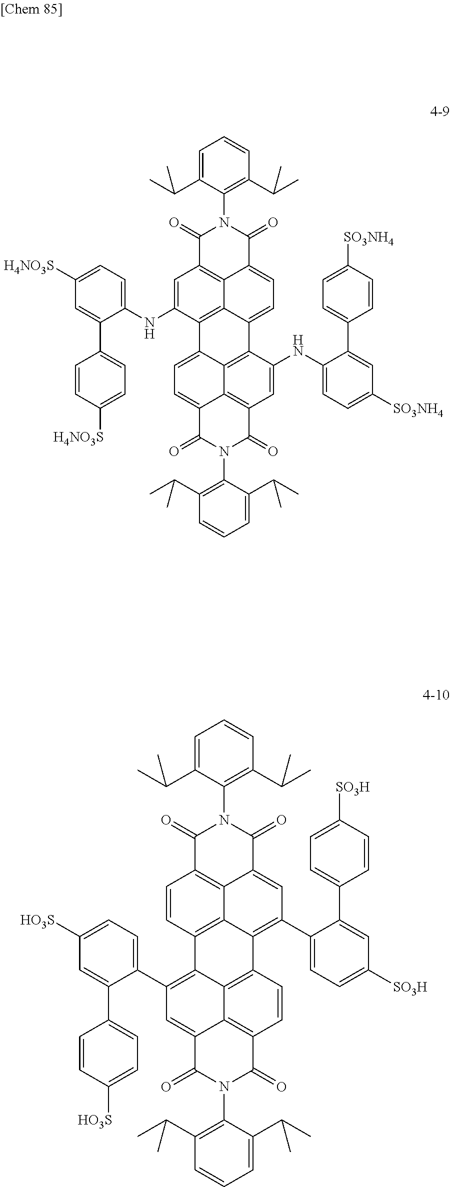

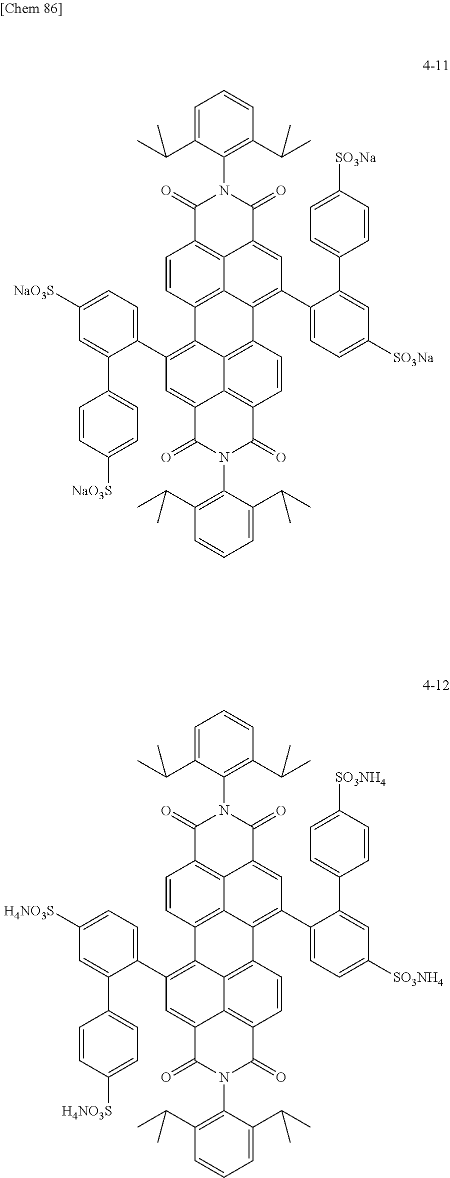

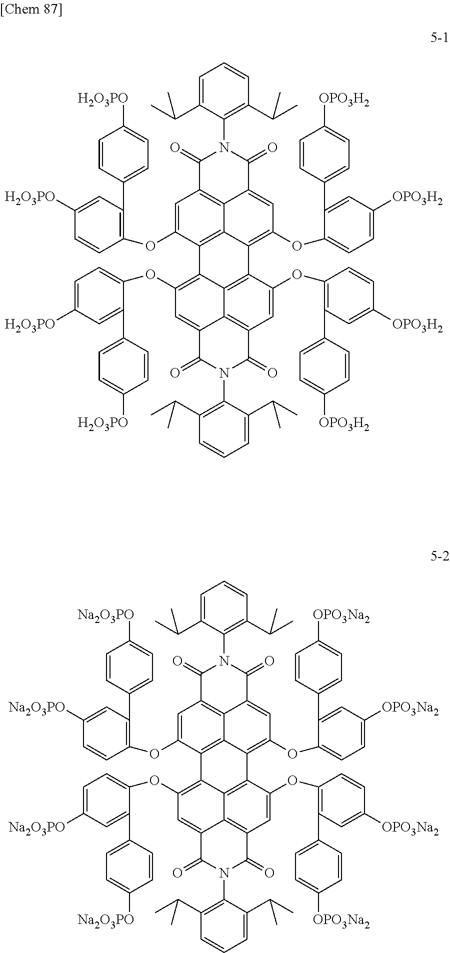

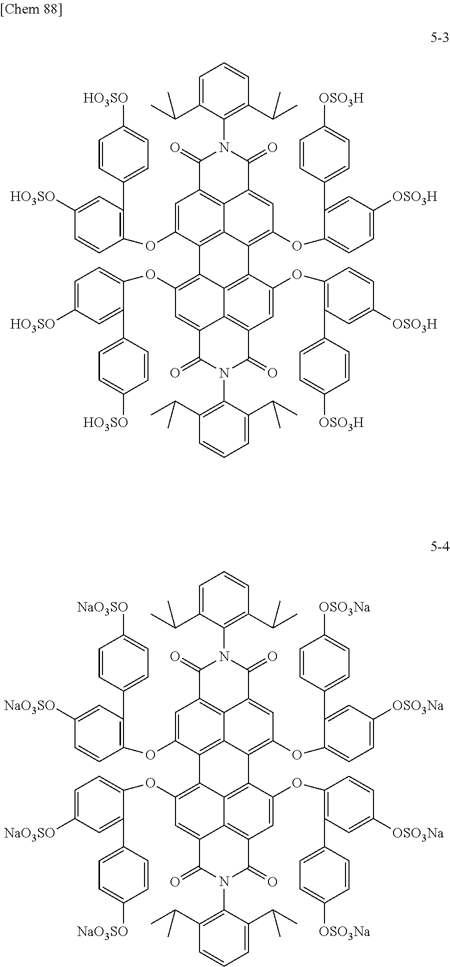

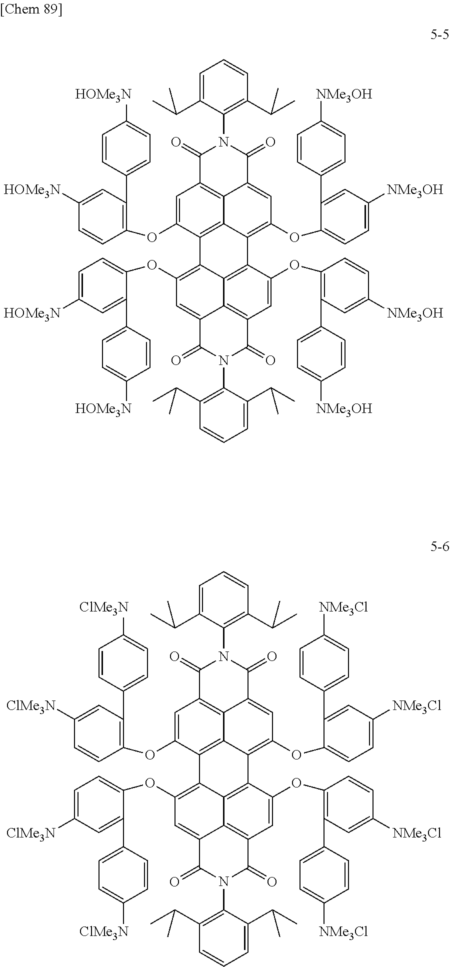

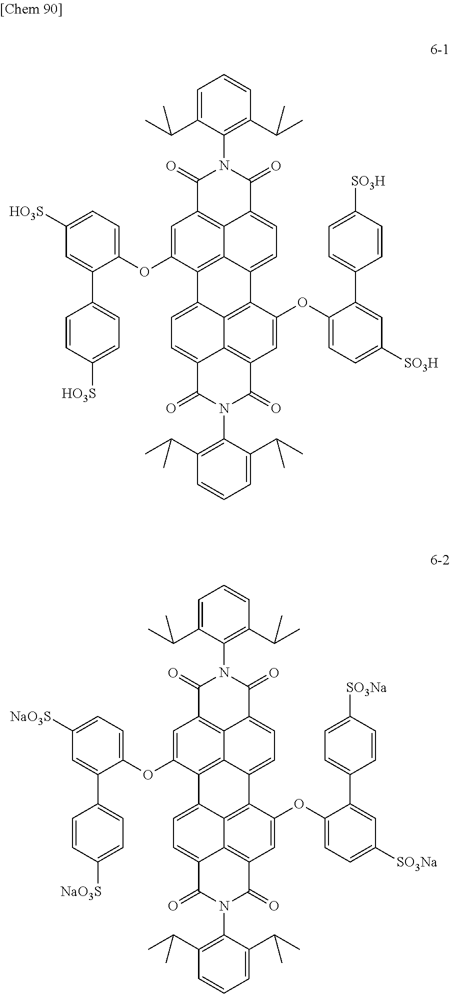

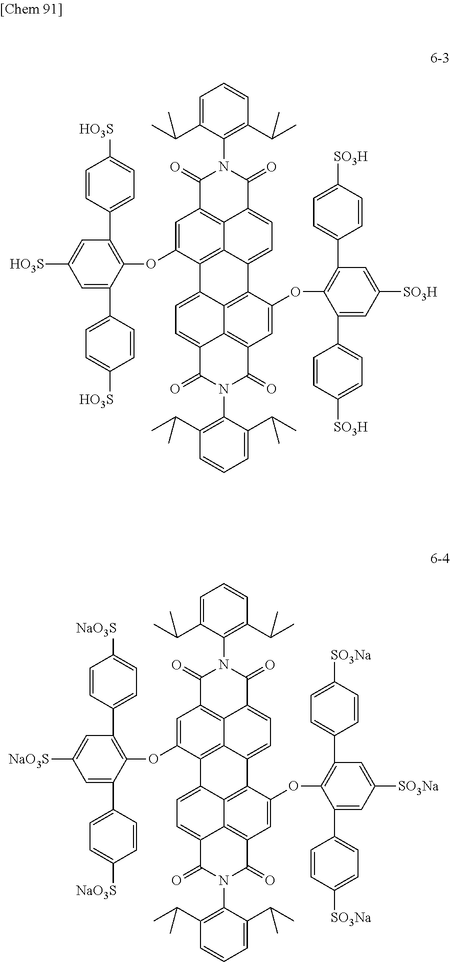

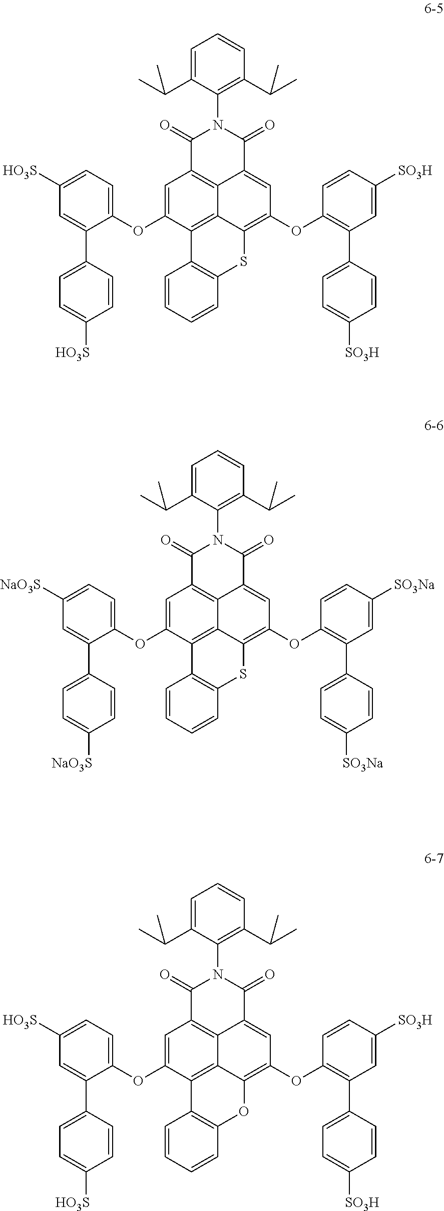

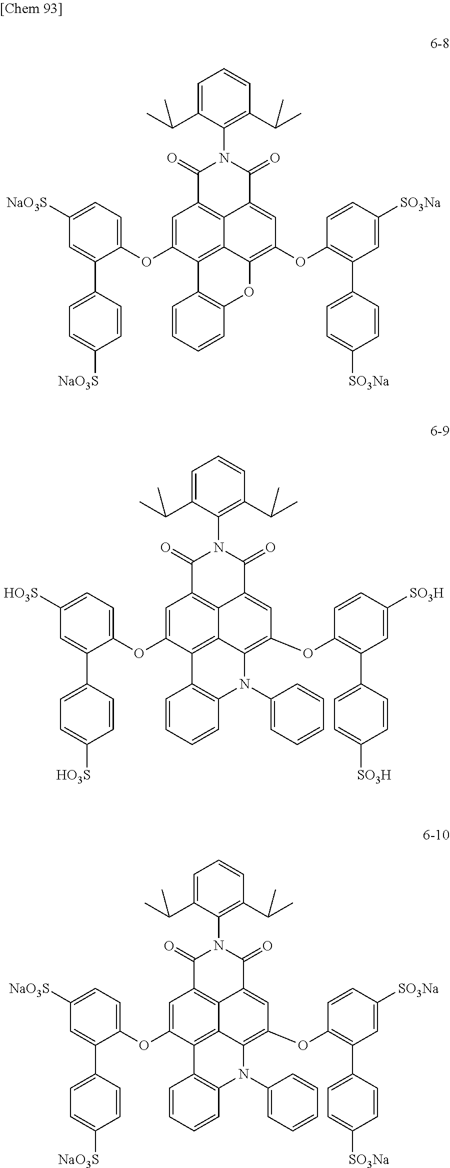

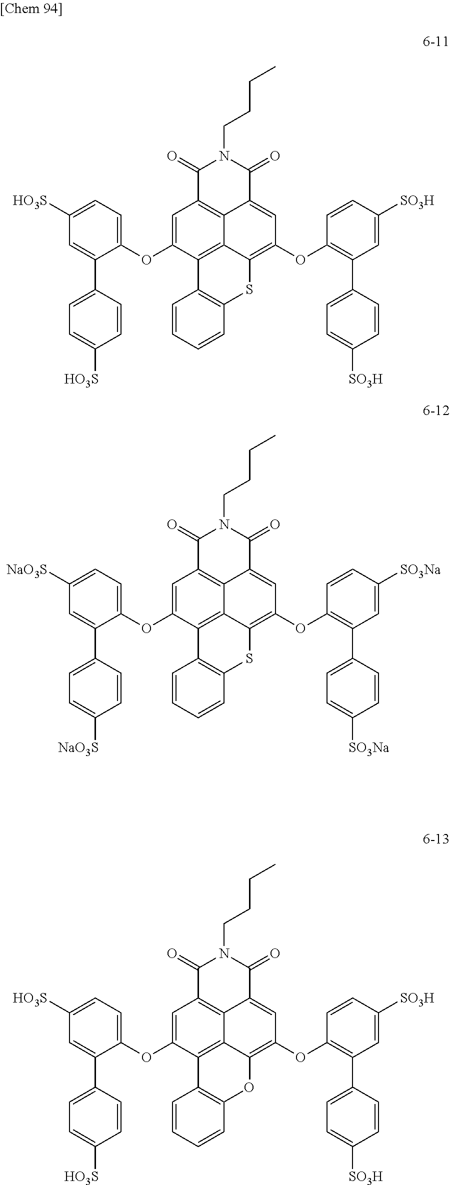

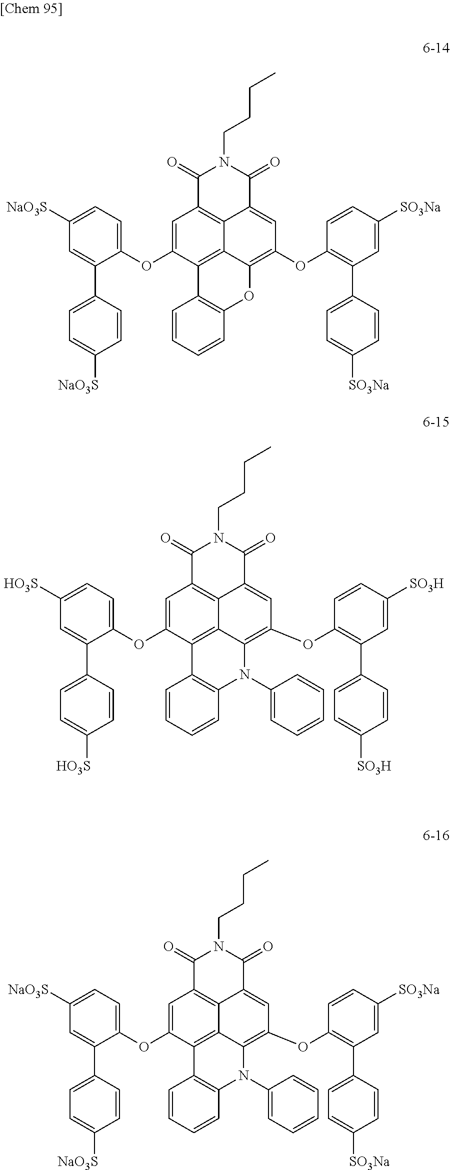

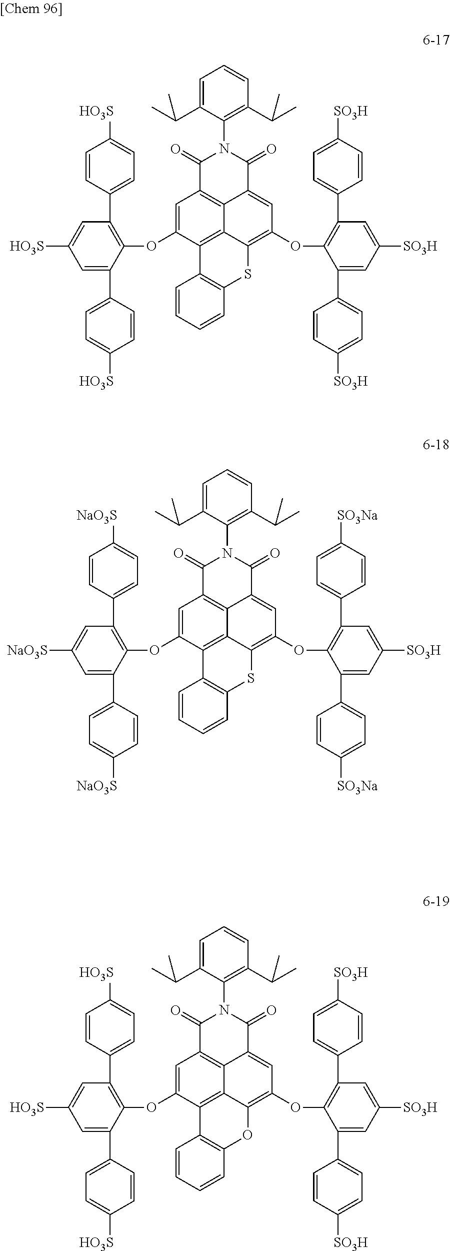

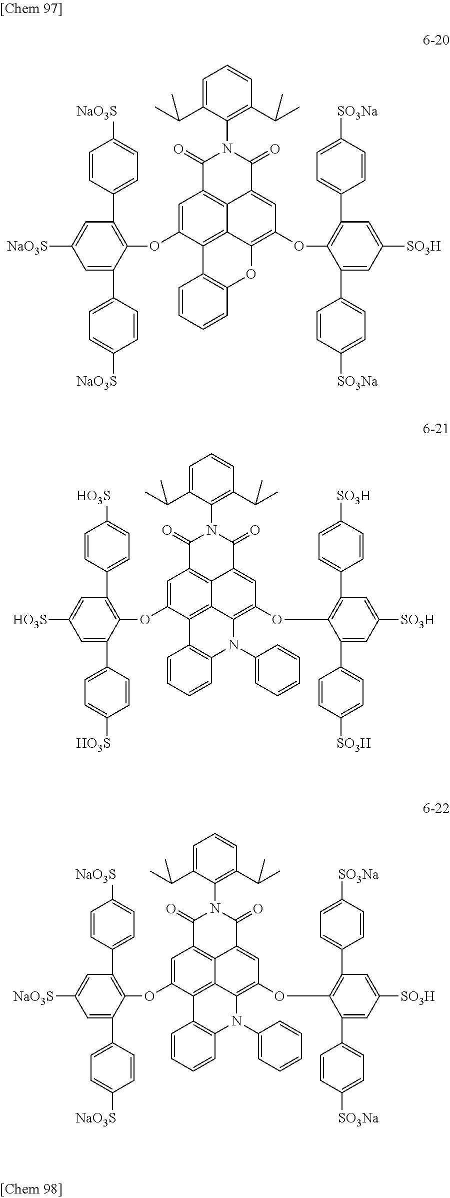

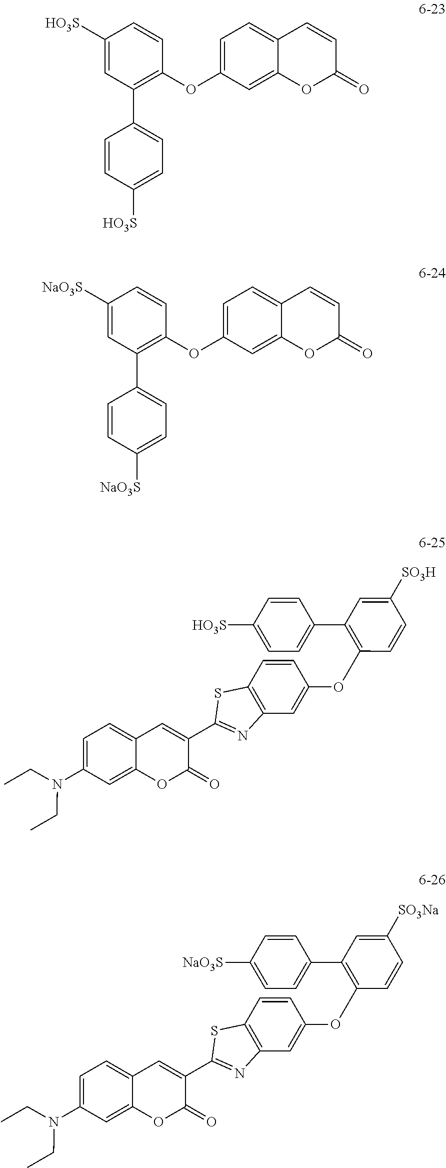

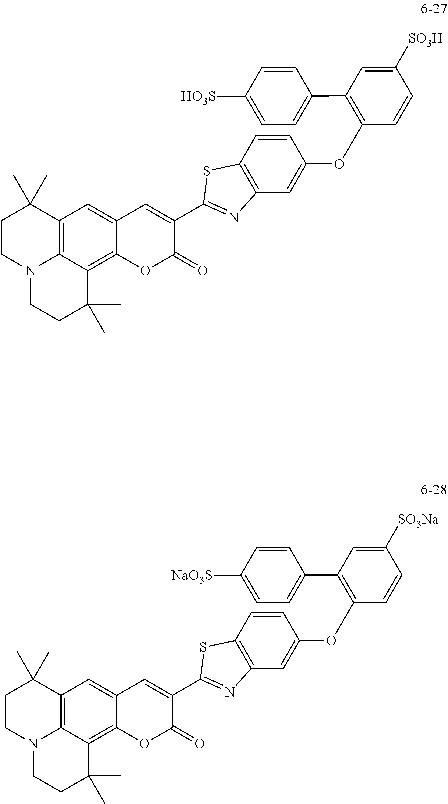

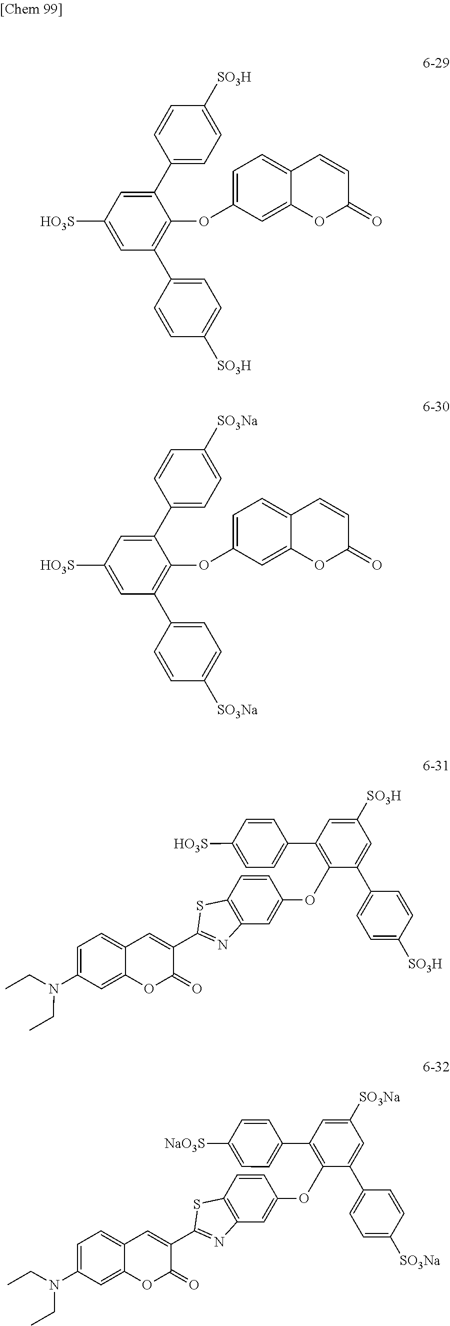

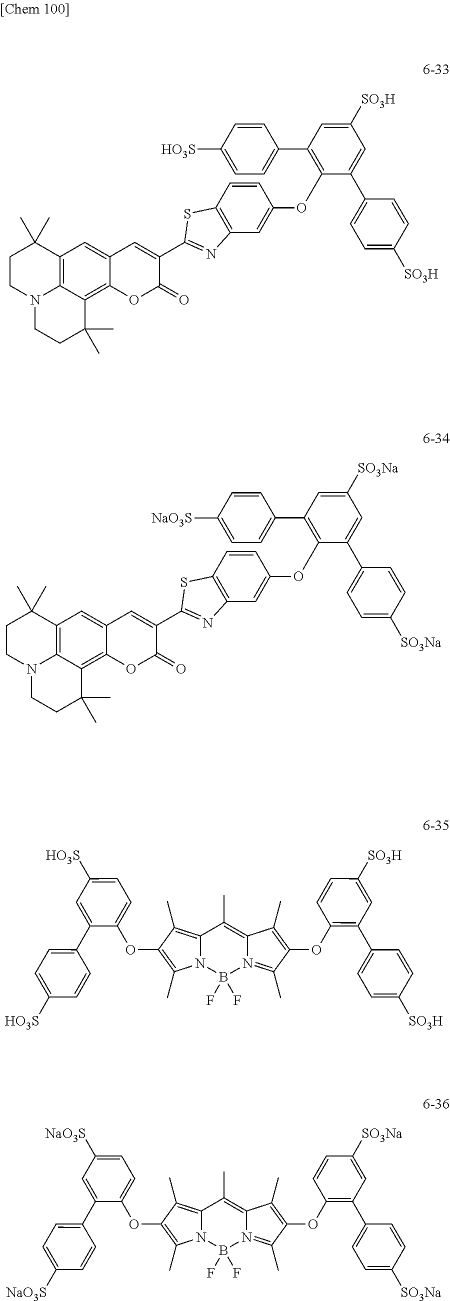

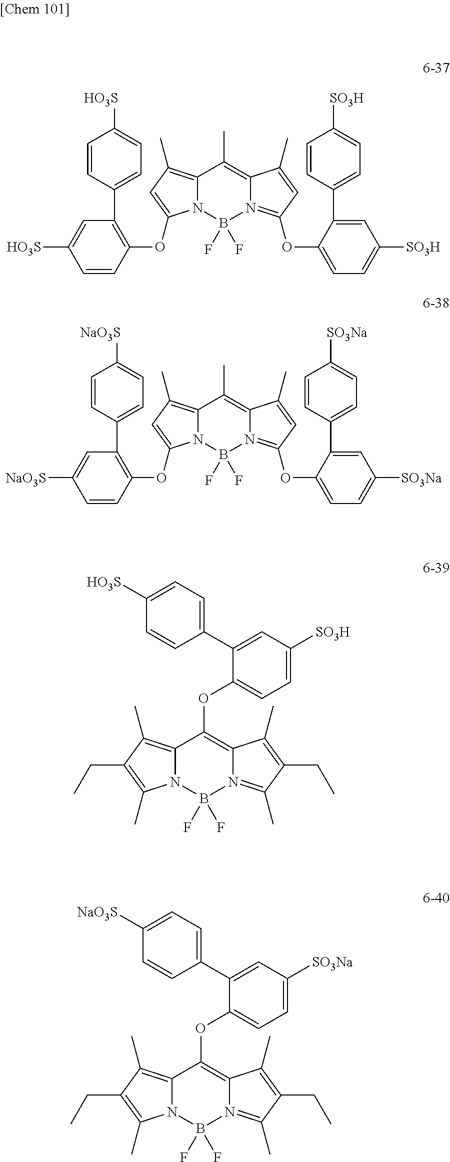

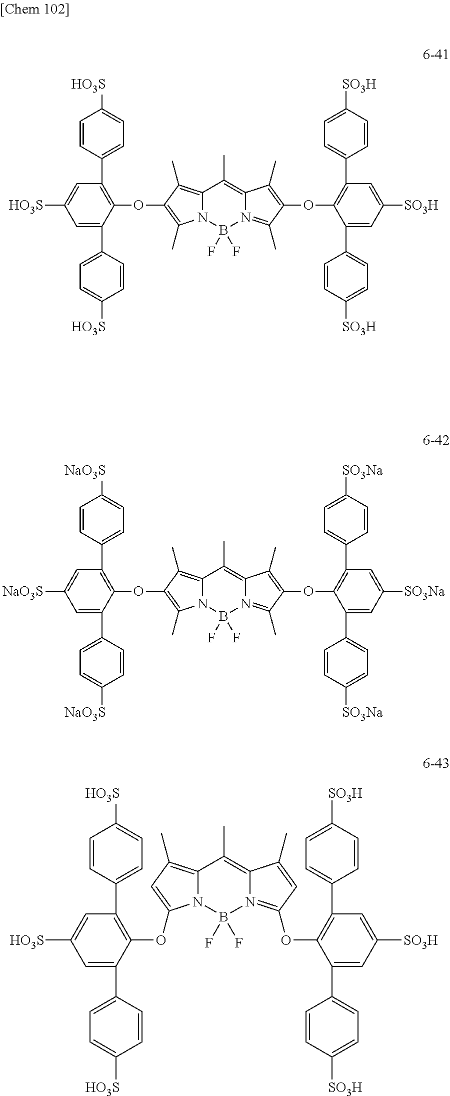

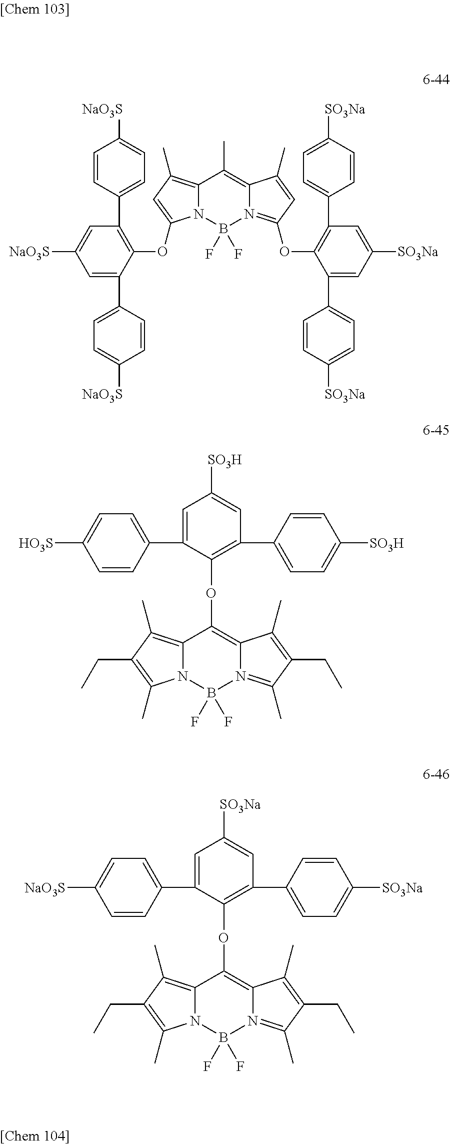

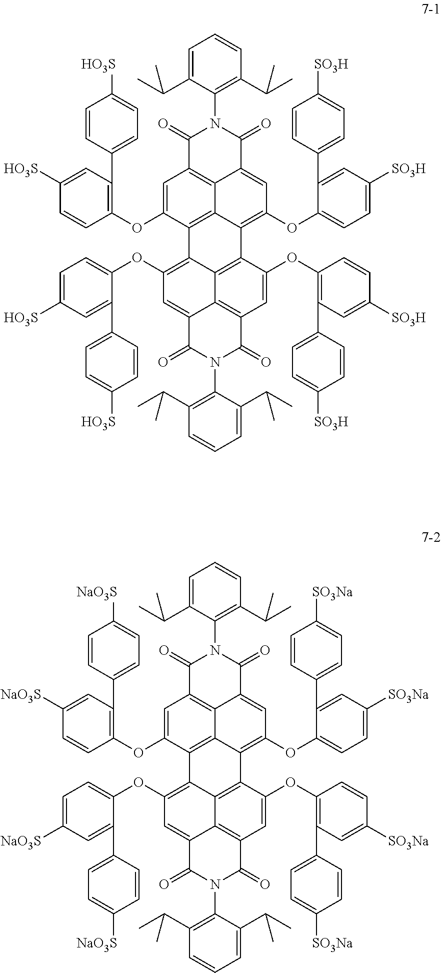

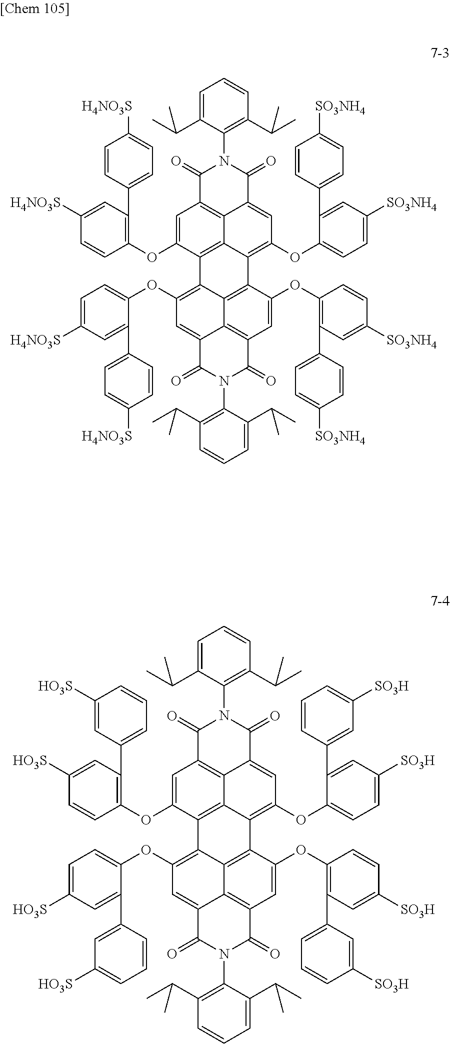

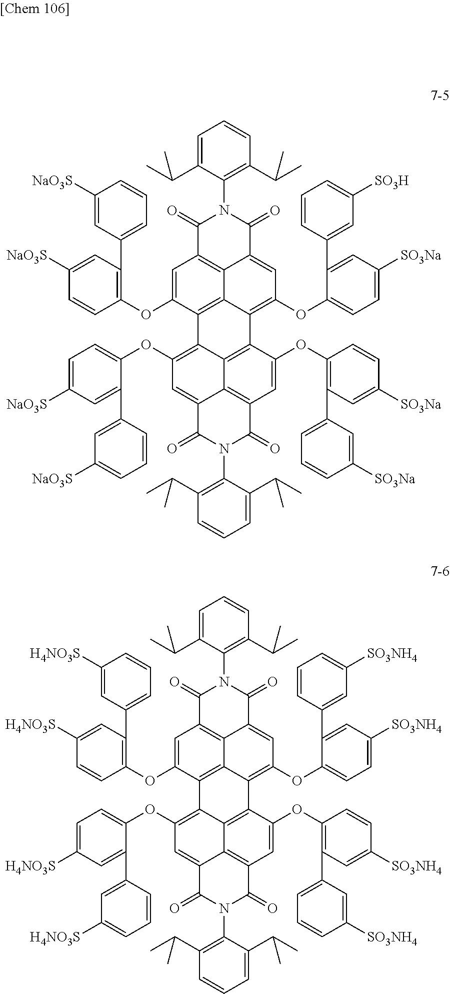

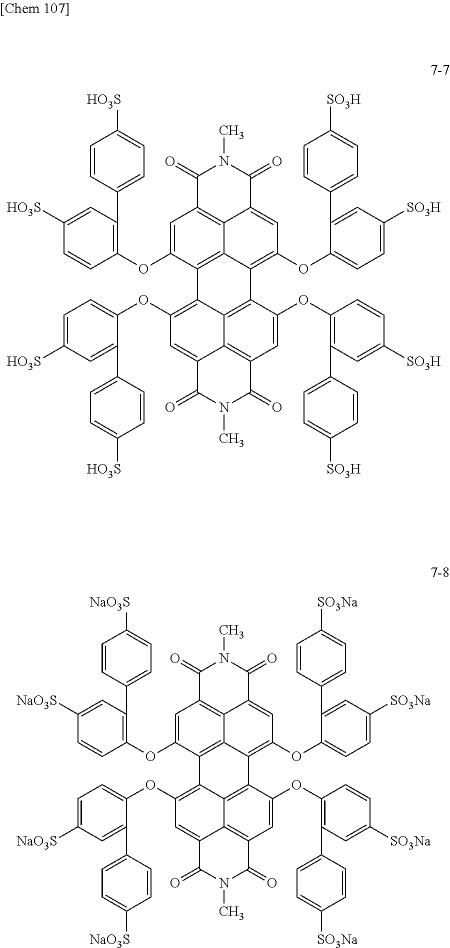

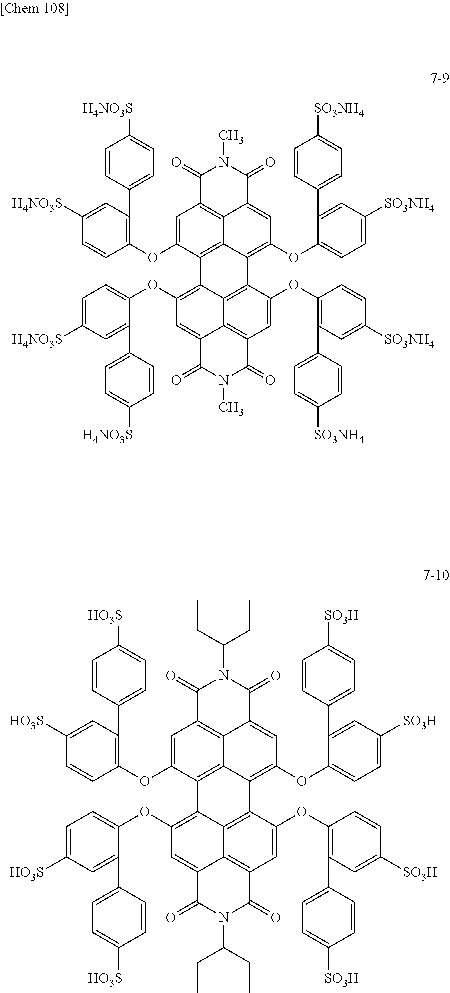

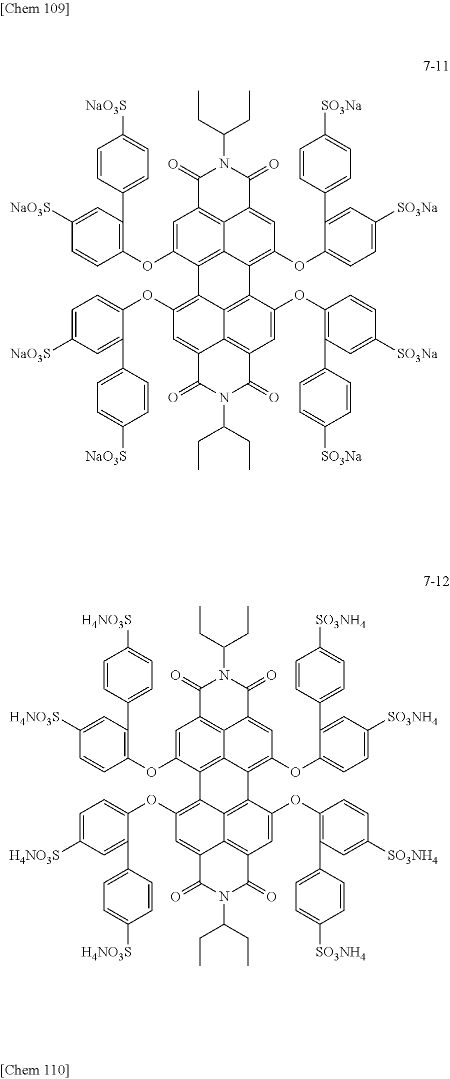

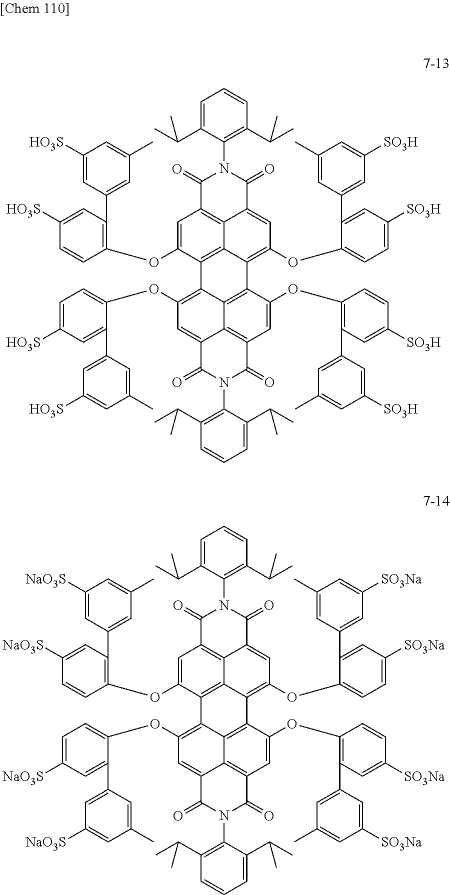

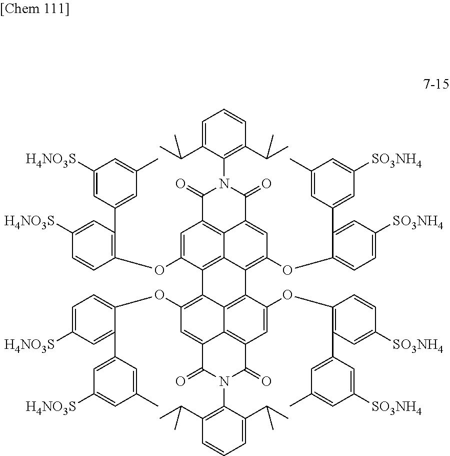

### (Synthesis of Compound (3))

The synthesis of the compound (3) will be described with reference to a case where X of the compound (3) is a sulfo group. The synthesis of the compound (3) in which X is a sulfo group can be carried out, for example, by introducing one sulfo group into each benzene ring by sulfonating a compound (3) precursor in which X is a hydrogen atom in place of an ionic substituent in the general formula (3). As a result, it is possible to substitute a plurality of ionic substituents at a time, and thus the production efficiency is excellent.

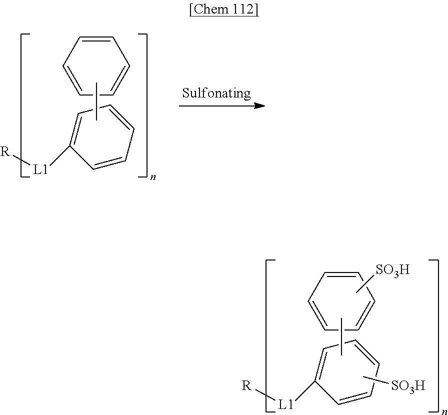

Wherein R represents a luminescent compound skeleton. L1 represents a single bond, an oxygen-atom, a sulfur-atom, a selenium-atom or a NH group. n represents an integer of 1 or more. In the above formula, R has the same meaning as R in the above general formula (3).

### Synthesis Example of Exemplified Compound 1-1

The synthesis scheme of exemplified compound 1-1 is shown below. Other exemplified compounds can be synthesized in the same manner. In the synthetic scheme, NMP represents N-methyl-2-pyrrolidone.

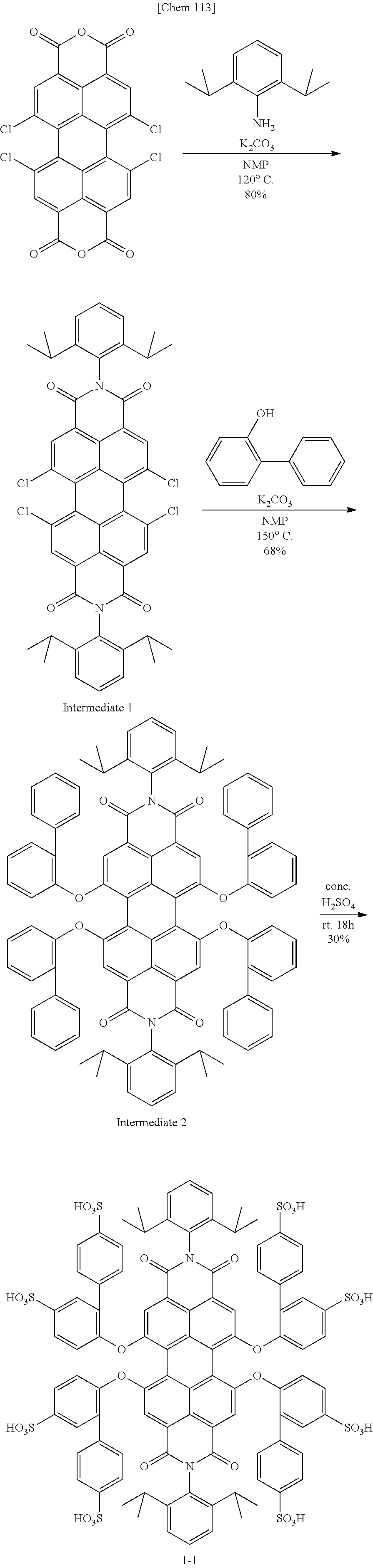

The first luminescent compound has been described above. In the present invention, one kind of the first luminescent compound may be used alone, or two or more kinds thereof may be used in combination. From the viewpoint of improving the transfer efficiency by limiting the energy-transfer path, it is preferable to use one kind of the first luminescent compound alone. When two or more kinds of the first luminescent compound are used in combination, a content of the first luminescent compound in the luminescent nanoparticle is the sum of the amounts.

### <Second Luminescent Compound>

The maximum luminescence wavelength λₑₘ₂ of the second luminescent compound is preferably within the above-described range. On the other hand, for the maximum absorption wavelength λ_{ab2}, considering the maximum luminescence wavelength λₑₘ₁ of the first luminescent compound and the maximum luminescence wavelength λₑₘ₂ of the second luminescent compound, it is generally preferably within the range of 500-900 nm, and more preferably within the range of 600-800 nm.

A molar ratio of the second luminescent compound to the first luminescent compound (second luminescent compound: the first luminescent compound) is preferably in the range of 1:2 to 1:200, more preferably 1:4 to 1:100, still more preferably 1:8 to 1:75, and still much more preferably 1:16 to 1:50.

A content of the second luminescent compound in the luminescent nanoparticle according to the present invention is preferably in the range of approximately 0.05 to 1% by mass, more preferably 0.1 to 0.5% by mass of the total amount of the luminescent nanoparticle according to the present invention, although it depends on the molar ratio of a content of the first luminescent compound and a content of the second luminescent compound.

The second luminescent compound is preferably a xanthene dye from the viewpoint of chemical and optical stability of the compound. The xanthene dye typically includes a compound having a structure represented by the following general formula (10).

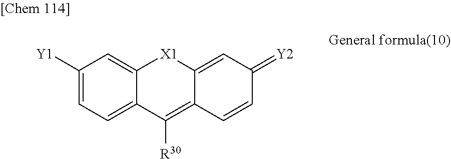

In Equation (10), X1 represents O, CR₂, SiR₂, P(═O)R, or BR₂. Each R independently represents a hydrogen atom or a substituent. Y1 represents an amino group or a hydroxy group, and Y2 represents an ammonium group or an oxygen atom. R³⁰ represents a hydrogen-atom or a substituent.

Specific examples of R when R in CR₂, SiR₂, P(═O)R, BR₂ represented by X1 is a substituent include the groups listed as the substituent that * may have in the general formula (1). The same applies when R³⁰ is a substituent.

When Y1 is an amino group, the amino group typically includes —NR₂, where R is a hydrogen atom or a substituent. Specific examples of R in the case where R is a substituent include the groups mentioned as the substituent which * may have in the general formula (1). The two R's may be bonded to each other to form a ring.

When Y2 is an ammonium group, the ammonium group typically include ═NR₂⁺, where R is a hydrogen atom or a substituent. Specific examples of R in the case where R is a substituent include the groups mentioned as the substituent which * may have in the general formula (1). The two R's may be bonded to each other to form a ring. R may be bonded to a carbon atom constituting a benzene ring to which a nitrogen atom is bonded to form a ring.

When Y2 is an ammonium group, the compound (10) has a counter-anion in or out of the molecule. When the counter-anion is present in the molecule, a configuration in which R³⁰ has the counter-anion is preferable. Examples of the counter anion include COO⁻, SO₃⁻ and the like. When the compound (10) has a counter anion outside the molecule, examples of the counter anion include I⁻, F⁻, Br⁻, Cl⁻, PF₆⁻, BF₄⁻, ClO₄⁻ and the like.

Among the xanthene dyes, the following compounds are particularly preferred.

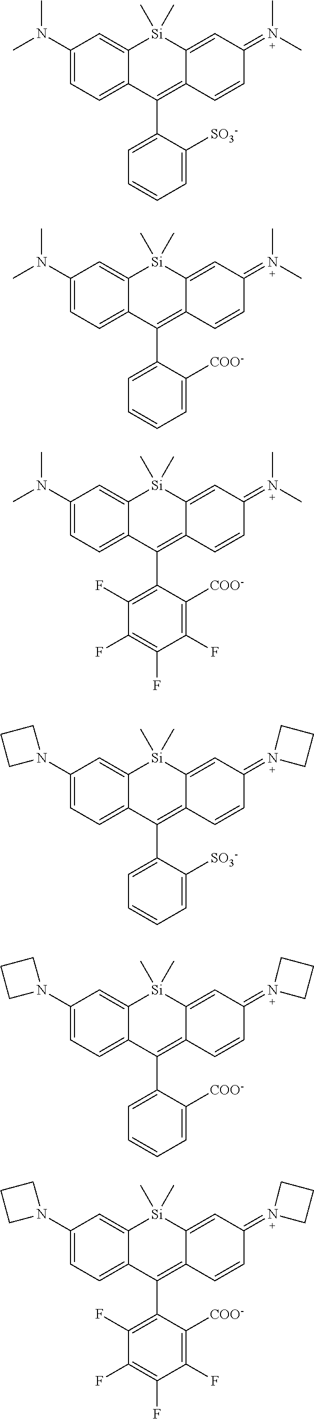

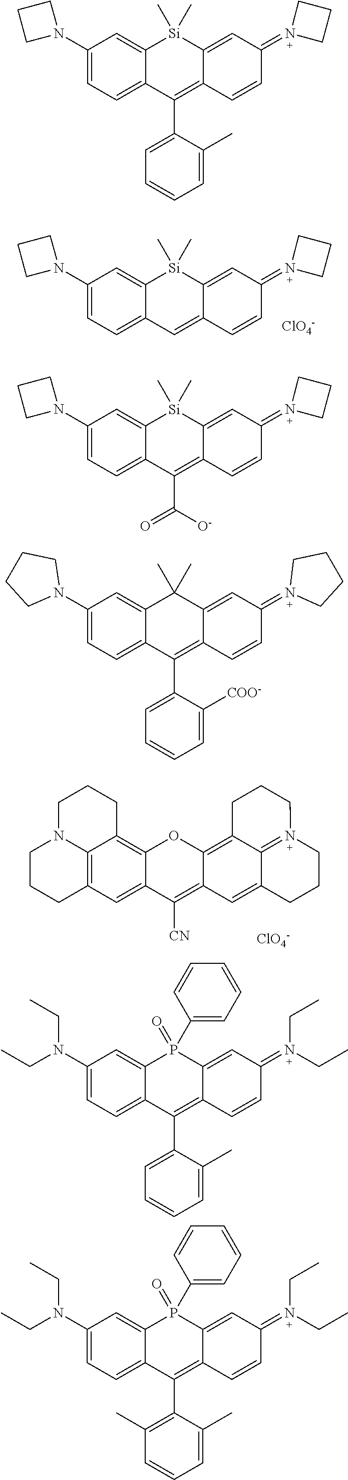

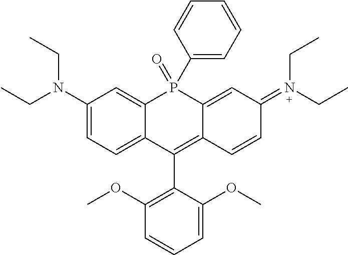

As the second luminescent compound, a cyanine dye, a squarylium dye, a dipyrromethene dye, an azadipiromethene dye, a terylene dye, a perylene dye other than the first luminescent compound, or the like may be used in addition to the xanthene dye. The structures of typical compounds of these dyes are shown below.

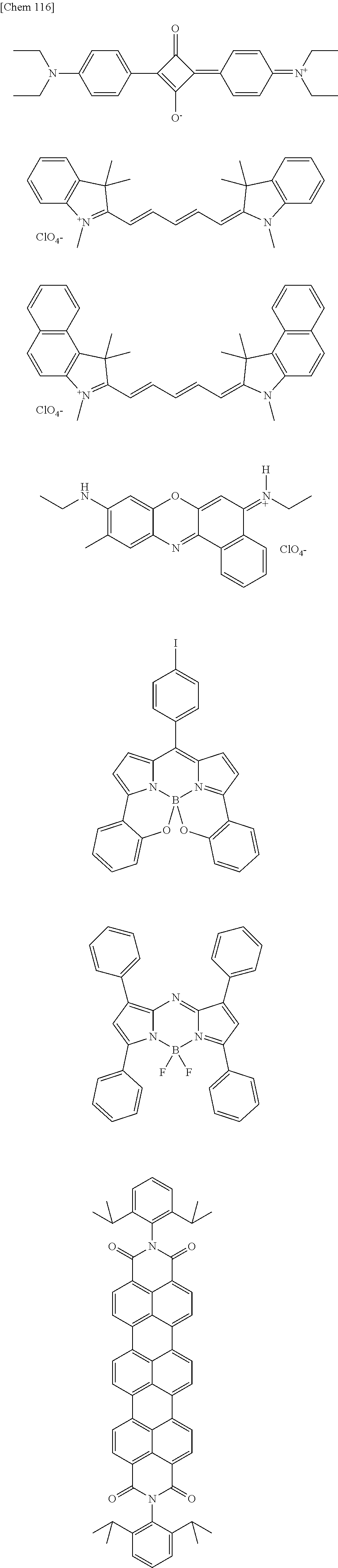

The second luminescent compound has been described above. In the present invention, one kind of the second luminescent compound may be used alone, or two or more kinds thereof may be used in combination. From the viewpoint of improving the transfer efficiency by limiting the energy-transfer path, it is preferable to use one of the second luminescent compound alone. When two or more kinds of the second luminescent compound are used in combination, a content of the first luminescent compound in the luminescent nanoparticle is the sum of the amounts.

### <Other Luminescent Compounds>

The luminescent nanoparticle according to the present invention may optionally include luminescent compounds other than the first luminescent compound and the second luminescent compound as long as the effect according to the present invention is not impaired. However, from the viewpoint of sufficiently achieving the advantages according to the present invention, it is preferable not to include the other luminescent compounds.

### <Binder>

The luminescent nanoparticle according to the present invention preferably includes a binder which works as a fixing material or a binding material, from the viewpoint of being able to impart a specific function, via the binder, to a surface of the luminescent nanoparticle.

When the binder is included, a content of the binder to the total amount of the luminescent nanoparticle is an amount obtained by removing the total amount of the first luminescent compound, the second luminescent compound, and the other luminescent compounds from the total amount of the luminescent nanoparticle, for example, 9 to 95% by mass, preferably 10 to 95% by mass, more preferably 19 to 90% by mass, and still more preferably 20 to 90% by mass.

The binder is preferably a hydrolyzed condensate of an organic resin or a metal alkoxide having a molecular weight of 300 or more and containing a carbon atom in the main chain.

Specific examples of the organic resin include polyolefin resins such as polypropylene, polymethylpentene, and polycyclohexylene dimethylene terephthalate (PCT), polyamides, polyethylene terephthalate, polybutylene terephthalate, polyphenylene sulfide, polycarbonate, ABS resins, AS resins, acrylic resins, amino resins, polyester resins, epoxy resins, mixed resins of acrylic resins and amino resins, polyester resins, and amino resins, cellulose resins, polyethylene resins, polypropylene resins, polystyrene resins, polyvinyl chloride resins, polymethacrylate resins, polyacrylonitrile resins, polyacrylamide resins, polyalcohol resins, polyacetate allyl resins, polyoxymethylene resins, poly-n-butyl isocyanate resin, polyethylene oxide resin, 6-nylon resin, poly-β-oxypropionic acid ester resin, phenolic resins, urea resins, melamine resins, alkyd melamine resins, unsaturated polyester resins, polyvinyl alcohol resins, poly(N-vinyl formamide) resins, poly(N-vinyl isobutylamide) resins, polyacrylic acid resins, poly(N-isopropylacrylamide) resins, poly(N-vinyl pyrrolidinone) resins, polyhydroxyethyl methacrylate resins, polyoxyethylene methacrylate resins, polyethylene glycol dimethyl ether resins, and polystyrene sulfonic acid resins.

Specific examples of the metal in the metal alkoxide include magnesium, calcium, strontium, scandium, yttrium, ruthenium, lawrencium, lanthanum, titanium, zirconium, hafnium, cerium, vanadium, niobium, tantalum, chromium, molybdenum, tungsten, manganese, ruthenium, cobalt, rhodium, iridium, nickel, platinum, palladium, copper, silver, gold, zinc, aluminum, gallium, indium, silicon, germanium, and tin.

The luminescent nanoparticle preferably has a hydrophilic group on a surface thereof. The hydrophilic group of the luminescent nanoparticle may be a hydrophilic group of the first luminescent compound or the second luminescent compound, or may be a hydrophilic group of the binder. In the luminescent nanoparticle according to the present invention having a hydrophilic group on the surface, aggregation of the particles is preferably suppressed so that the particles can be dispersed in water.

### (Binders Having Hydrophilic Group)

The hydrophilic group included in the binder is preferably an atomic group having a strong interaction with water, and specifically, —OH, —SH, —COOH, —C(═O)H, —S(═O)₂(═O) OH, —S(═O) NH₂, —S(═O)₂NH₂, —P(═O)(OH)₃, —P(═O)R(OH)₂, —P(═O)R²(OH), —P(OH)₃, —P(═O)(NH₂)₃, —P(═O)R(NH₂)₂, —P(═O)R²(NH₂), —P(NH₂)₃, —O(C═O) OH, —NH₂, —NHR, —NHCONH₂, —NHCONHR, —NHCOOH, —Si(OH)₃, —Si(R)(OH)₂, —Si(R)₂OH, —Ge(OH)₃, —Ge(R) (OH)₂, —Ge(R)₂OH, —Ti(OH)₃, —Ti(R)(OH)₂, —Ti(R)₂OH, —Si(NH₂)₃, —Si(R)(NH₂)₂, —B(OH)₂, —O—B(OH)₂, —B(NH₂)₂, —NHB(OH)₂, a polyethyleneglycol group, or the like. Each R independently represents hydrogen or an alkyl group having 1 to 20 carbon atoms. In addition, an NHS group, a maleimide group, or the like may be also the hydrophilic group exhibiting hydrophilicity.

Specific examples of the binder having the hydrophilic group include a urea resin, a melamine resin, a polyvinyl alcohol resin, a poly(N-vinyl formamide) resin, a poly(N-vinyl isobutylamide) resin, a polyacrylic acid resin, a polyacrylamide resin, a poly(N-isopropylacrylamide) resin, a poly(N-vinyl pyrrolidinone) resin, a polyhydroxyethyl methacrylate resin, a polyoxyethylene methacrylate resin, a polyethylene glycol dimethyl ether resin, and a polystyrene sulfonic acid resin.

Hydrolysis condensates of metal alkoxides can also be the binder having the hydrophilic group. The metal alkoxide is preferably a titanium alkoxide, a zirconium alkoxide, a silicon alkoxide, or the like. Titania, zirconia, and silica are formed from titanium alkoxides, zirconium alkoxides, and silicon alkoxides as hydrolytic condensates, respectively. In the luminescent nanoparticle according to the present invention, melamine resin or silica is preferably used as the binder.

### (Thermosetting Resin)

The binder according to the present invention may be a thermosetting resin. For example, from the viewpoint that a luminescent compound is hardly eluted in a penetration step using an organic solvent such as xylene, an organic resin including a thermosetting resin such as a melamine resin, which is capable of immobilizing a luminescent compound inside a dense cross-linked structure, is preferable.

Examples of the thermosetting resin include those containing a structural unit formed from at least one monomer selected from the group consisting of melamine, urea, guanamines (including benzoguanamine, acetoguanamine, and the like), and derivatives thereof. Any one of these monomers may be used alone, or two or more of these monomers may be used in combination. If desired, a comonomer other than one or two or more of the above compounds may be used in combination.

Specific examples of the thermosetting resin include melamine-formaldehyde resins and urea-formaldehyde resins.

As a raw material of the thermosetting resin, not only the above-described monomer itself but also a prepolymer obtained by reacting the monomer with a compound such as formaldehyde or another crosslinking agent in advance may be used. For example, in the preparation of melamine-formaldehyde resins, methylolmelamine prepared by condensing melamine and formaldehyde under alkaline conditions is generally used as a prepolymer, and the compound may be further alkyl etherified (methylation to improve stability in water, butylation to improve solubility in organic solvent, and the like).

Further, the thermosetting resin may be one in which at least a part of hydrogen contained in the structural unit is replaced with a substituent having a charge or a substituent capable of forming a covalent bond. Such a thermosetting resin can be synthesized by using a (derivatized) monomer in which at least one hydrogen is replaced with the above-described substituent as a raw material by a known method.

Such a thermosetting resin can be synthesized according to a known method. For example, the melamine-formaldehyde resin can be synthesized by adding a reaction accelerator such as an acid, if necessary, and then heating and polycondensing the methylol melamine prepared in advance as described above.

### (Thermoplastic Resin)

The binder according to the invention may be a thermoplastic resin. Examples of the thermoplastic resin include those containing a structural unit formed from at least one monofunctional monomer selected from the group consisting of acrylic acid, methacrylic acid and alkyl esters thereof, acrylonitrile, and derivatives thereof (a group involved in a polymerization reaction in one molecule, in the above example, a monomer having one vinyl group). Any one of these monomers may be used alone, or two or more of these monomers may be used in combination.

If desired, a comonomer other than one or two or more of the above compounds may be used in combination. The thermoplastic resin may include a structural unit formed from a polyfunctional monomer such as divinylbenzene (a group involved in a polymerization reaction in one molecule, in the above example, a monomer having two or more vinyl groups), that is, a crosslinking site. Examples thereof include crosslinked products of polymethylmethacrylate.

In addition, the thermoplastic resin may contain structural units having functional groups for surface-modifying luminescent nanoparticle according to the present invention. For example, by using a raw material as a monomer such as glycidyl methacrylate having an epoxy group, a luminescent nanoparticle having an epoxy group oriented to the surface can be prepared. The epoxy group can be converted to an amino group by reacting with an excess of aqueous ammonia. Various kinds of biomolecules can be introduced into the amino group formed in this manner according to a known method (via a molecule as a linker, if necessary).

### <Manufacturing Method of Luminescent Nanoparticle>

As a manufacturing method of a luminescent nanoparticle according to the present invention, there can be mentioned a method of forming particles having a diameter of the order of nanometers, in which luminescent compounds (luminescent compounds including a first luminescent compound and a second luminescent compound, and hereinafter, similarly, “luminescent compounds” indicates luminescent compounds including the first luminescent compound and the second luminescent compound) are fixed to an inside or a surface of a base made of a binder.

The method for preparing the luminescent nanoparticle is not particularly limited, but for example, it is possible to use a method in which luminescent compounds are incorporated into or on (co)polymer by adding luminescent compounds while (co)polymerizing (co)monomers for synthesizing a binder (for example, a thermoplastic resin or a thermosetting resin) forming a base for the luminescent nanoparticle. When the binder is a hydrolytic condensate of a metal alkoxide, it is possible to use a method, for example, in which while the metal alkoxide is subjected to hydrolytic condensation, a luminescent compound is added so that a luminescent compound is incorporated in the inside or on the surface of the hydrolytic condensate.

The luminescent nanoparticles according to the present invention can be prepared using the first luminescent compound and the second luminescent compound in accordance with, for example, a known polymerizing step or a hydrolyzing condensation step for various binders. Hereinafter, a manufacturing method will be described by taking a case where the binder is an organic resin as an example.

### (Polymerization Step)

The polymerization step is a step of heating a reaction mixture including the luminescent compounds, a resin raw material (monomer, oligomer or prepolymer), preferably surfactant and a polymerization reaction accelerator, to cause the polymerization reaction of the resin to proceed, thereby producing resin grains containing the luminescent compounds.

The order of addition of the components included in the reaction mixture is not particularly limited. Typically, a sequence is used in which surfactant is added to an aqueous solution of the luminescent compounds, followed by addition of resinous raw material, and finally addition of a polymerization accelerator. Alternatively, the order may be such that the resin raw material is added to the aqueous solution of surfactant, and then the aqueous solution of the luminescent compounds is added while the polymerization reaction accelerator is added to proceed the synthesis reaction of the resin grains. It should be noted that the concentration of an aqueous solution of particular luminescent compounds according to the present invention used in such a polymerization step can be adjusted to a relatively higher concentration (e.g., 2,500-10,000 μM) than that of a conventional aqueous solution of luminescent compounds.

Polymerization conditions (temperature/time, etc.) can be appropriately set, considering the type of resin, composition of raw material mixtures, etc.

The polymerization method is not particularly limited as long as it is a known polymerization method. Examples of known polymerization methods include bulk polymerization, emulsion polymerization, soap-free emulsion polymerization, seed polymerization, suspension polymerization, and the like. In the case of bulk polymerization, the resin particles having a desired particle size can be obtained by classifying the resin particles after pulverization. Emulsion polymerization is a polymerization method in which a medium such as water, a monomer which is difficult to dissolve in the medium, and an emulsifying agent (surfactant) are mixed, and a polymerization initiator which is soluble in the medium is added thereto. Particle diameter obtained is characterized in that the variation is small.

“Soap-free emulsion polymerization” is an emulsion polymerization without an emulsifier. It is characterized in that particles having a uniform diameter can be obtained. The seed polymerization is a polymerization performed by adding seed particles separately produced at the time of initiation of polymerization. It is characterized in that particle diameters and particle diameter distributions and quantities (numbers of particles) are arbitrarily determined as the seed particles, and they are polymerized with the aim of the desired particle diameter and the particle diameter distribution. The suspension polymerization is a polymerization process in which a monomer and solvent are mechanically stirred and suspended. It is characterized in that particle diameter can be obtained with small and well-formed grains.

As a specific example, for the synthesis of thermosetting resins such as melamine resins, the reaction temperature is usually 70 to 200° C. and the reaction time is usually 20 to 120 minutes. It should be noted that the reacting temperature should be a temperature at which the performance of the luminescent compounds does not deteriorate (within the heat-resistant temperature range). The heating may be performed in a plurality of stages, and for example, the reaction may be performed at a relatively low temperature for a fixed time, and then the reaction may be performed at a relatively low temperature for a fixed time by raising the temperature.

After the polymerization reaction is completed, the excess resin raw material, the luminescent compounds, and surfactant may be removed from the reaction solution, and the resulting luminescent nanoparticle may be recovered and purified. For example, the reaction solution is centrifuged to remove the supernatant containing impurities, and then ultrapure water is added thereto, irradiated with ultrasonic waves, dispersed again, and washed. These operations are preferably repeated a plurality of times until no light absorption or luminescence originating from the resin or the luminescent compounds is observed in the supernatant.

The luminescent nanoparticle in which the thermosetting resin is used can be produced basically according to an emulsion polymerization method, but is preferably produced by a polymerization process as described above using surfactant and a polymerization reaction accelerator. In the luminescent nanoparticle obtained by such a production process, the luminescent compounds are immobilized in a state in which most of the luminescent compounds or preferably substantially all of the luminescent compounds are contained in the resin particles, but it is not excluded that the luminescent compounds are immobilized in a state in which some of the luminescent compounds are bound or adhered to the surface of the resin particles.

In addition, in the condition in which the luminescent compounds are contained, it is not limited to what kind of chemical or physical action by which the luminescent compounds are immobilized on the resin-particle. In the present invention, it is not necessary to cause the resin raw material and the luminescent compounds to be covalently bonded in advance or provide a derivatization step for introducing a positively charged substituent into the resin raw material, prior to the polymerizing step (even if such a step is not used, an luminescent nanoparticle having excellent luminescent strength and light resistance can be obtained), but it is not excluded that such a step is used in combination if desired.

### (Surfactant)

As surfactant, a known emulsifying agent for emulsion polymerization can be used. Examples of surfactant include anionic, nonionic, and cationic. When synthesizing a (cationic) thermoset having a positively charged substituent or moiety, an anionic or nonionic surfactant is preferably used. Conversely, when a thermosetting resin having a negatively charged substituent or moiety (anionic) is synthesized, it is preferable to use a cationic or nonionic surfactant.

Examples of the anionic surfactant include sodium dodecylbenzene sulfonate (product name “Neoperex” series, Kao Corporation). Examples of the nonionic surfactant include polyoxyethylene alkyl ether compounds (product names “Emulgen” series, Kao Corporation), polyvinylpyrrolidone (PVP), and polyvinyl alcohol (PVA). Examples of the cationic surfactant include dodecyltrimethylammonium bromide.

By adjusting the quantity of surfactant added, particle diameters of the resinous particles can be adjusted, and the coefficient of variation of particle diameters can be reduced, that is, a luminescent nanoparticle having uniform particle size can be manufactured. A content of surfactant is, for example, from 10 to 60% by mass, based on the resinous raw material, or from 0.1 to 3.0% by mass, based on the total raw material content. When the amount of surfactant added is increased, the particle size tends to decrease, and conversely, when the amount of surfactant added is decreased, the particle size tends to increase.

### (Polymerization Reaction Accelerator)

The polymerization reaction accelerator has a function of accelerating a polycondensation reaction of a thermosetting resin such as a melamine resin, and imparting a proton (H⁺) to a functional group such as an amino group contained in the resin or the luminescent compounds to thereby cause a charge, thereby facilitating an electrostatic interaction. The reaction of the thermosetting resin proceeds only by heating, but when the polymerization reaction accelerator is added, the reaction proceeds at a lower temperature, so that the reaction and the performance can be controlled. Examples of such a polymerization reaction accelerator include acids such as formic acid, acetic acid, sulfuric acid, paratoluenesulfonic acid, and dodecylbenzenesulfonic acid. When the luminescent compounds are a compound having a carboxy-group or a sulfo-group, the luminescent compounds can also donate protons in the same manner as described above.

### [Luminescent Labeling Material for Pathological Diagnosis Use]

A luminescent labeling material for pathological diagnosis use according to the present invention is characterized in that the luminescent nanoparticle according to the present invention described above is used. Specifically, target directing ligands are covalently bound to a surface of the luminescent nanoparticle according to the present invention in the luminescent labeling material for pathological diagnosis use according to the present invention.

The use of the luminescent nanoparticle according to the present invention is not particularly limited, but typically includes a use as a luminescent labeling material for pathological diagnosis use for labeling a substance to be detected contained in a sample (tissue section) and allowing fluorescence observation in immunodyeing. That is, the luminescent nanoparticle according to the present invention as described above is preferably used as a complex (conjugate) by linking target directing ligands according to embodiments of immunodyeing.

The substance to be detected is not particularly limited, but in pathological diagnosis, an antigen according to the purpose is generally selected. For example, HER2 can be used as a substance to be detected in the pathodiagnosis of breast cancer. In addition, the substance to be detected may not be a substance specific to a living body. For example, the substance to be detected may be a drug.

### Target Directing Ligand

In the present invention, the term “target directing ligand” refers to a molecule having specific binding to a specific tissue or cell (substance to be detected). The target directing ligand according to the present invention is preferably a molecule selected from the group consisting of an antibody, an organelle affinity substance, and a protein having binding to a sugar chain, from the viewpoint of suppressing non-specific adsorption.

The type of the target directing ligand is not particularly limited, and an optimum one can be selected according to the purpose. Specific examples of the target directing ligand include the following.

A first examples of the target directing ligand is a primary antibody (an antibody that specifically binds to a substance to be detected). The luminescent labeling material for pathological diagnosis use, in which the target directing ligand is a primary antibody, is directly bound to the substance to be detected, which makes it possible to fluorescently label the substance to be detected (primary antibody method).

A second example of the target directing ligand is a secondary antibody (an antibody that binds to the primary antibody). For example, if the primary antibody is an antibody produced from a rabbit (IgG), the secondary antibody will be an anti-rabbit IgG antibody. The luminescent labeling material for pathological diagnosis use, in which the target directing ligand is a secondary antibody, is bound to the primary antibody bound to the substance to be detected, which makes it possible to fluorescently label the substance to be detected indirectly (secondary antibody method).

A third example of the target directing ligand is avidin, streptavidin or biotin. For example, when the luminescent labeling material for pathological diagnosis use, in which the target directing ligand is avidin or streptavidin, is used, a secondary antibody-biotin complex is used in combination. The secondary antibody-biotin complex is bound to the primary antibody bound to the substance to be detected, and the luminescent labeling material for pathological diagnosis use, in which avidin or streptavidin is the target directing ligand, is bound to the complex, which makes it possible to fluorescently label the substance to be detected indirectly ((biotin-avidin method or sandwich method). Conversely, the luminescent labeling material for pathological diagnosis use, in which the target directing ligand is biotin, can also be used in conjunction with a secondary antibody-avidin complex or a secondary antibody-streptavidin.

The primary antibody may be selected to specifically bind to the selected substance to be detected. For example, when the substance to be detected is HER2, an anti HER2 monoclonal antibody can be used as the primary antibody. Such a primary antibody (monoclonal antibody) can be produced by a general method using a mouse, a rabbit, a cow, a goat, a sheep, a dog, a chicken, or the like as an immunized animal.

The secondary antibody may be selected to bind to the selected primary antibody. For example, when the primary antibody is a rabbit anti HER2 monoclonal antibody, an anti-rabbit IgG antibody can be used as the secondary antibody. Such a secondary antibody can also be produced by a general method.

In addition, it is also possible to use a nucleic acid molecule having a nucleotide sequence complementary to the nucleic acid molecule as a target directing ligand corresponding to the substance to be detected as a nucleic acid molecule.

The luminescent labeling material for pathological diagnosis use may be produced by any known method. For example, amidation by reaction of an amine with a carboxylic acid, sulfidation by reaction of maleimide with a thiol, imination by reaction of an aldehyde with an amine, and amination by reaction of an epoxy with an amine can be used. The functional group involved in such a reaction may be a functional group previously present on the surface of the luminescent nanoparticle (a functional group derived from the raw material monomer of the binder), a functional group obtained by converting the functional group present on the surface of the luminescent nanoparticle according to a known method, or a functional group introduced by surface modification or the like. Appropriate linker molecules may be utilized if desired.

In another aspect according to the present invention there is provided a kit for tissue immune dyeing using the luminescent nanoparticle according to the present invention. The kit includes at least the luminescent labeling material for pathological diagnosis use according to the present invention, the luminescent nanoparticle according to the present invention, the target directing ligand, and reagents. The kit may further optionally include a primary antibody, a secondary antibody, the other target directing ligands (e.g., biotin) used in combination with the target directing ligand (e.g., streptavidin), reagents to form the desired complex, other reagents used for immunohistodyeing, and the like.

### <Manufacturing Process of Luminescent Labeling Material for Pathological Diagnosis Use>

In the technical field to which the present invention belongs, various techniques for producing luminescent labeling material for pathological diagnosis use by covalently attaching a luminescent labeling body (luminescent nanoparticle according to the present invention) to a target directing ligand or the like are known, and such techniques can also be used for the present invention.

Reactions that occur among reactive functional groups such as, for example, carboxy, amino, aldehyde, thiol, maleimide groups can be used to covalently bond the luminescent labeling material for pathological diagnosis use (one reactive functional group present on its surface) to the target directing ligand (the other reactive functional group present in its molecule). In addition, when the functional groups of these compounds cannot be directly bonded to each other, they can also be bonded to each other through a “linker molecule” having a predetermined functional group at both ends of the molecule. Such a reaction can be carried out by adding necessary reagents and allowing a predetermined period of time to elapse.

Specific examples include methods in which a silane coupling agent (for example, aminopropyltrimethoxy silane) is reacted with a luminescent nanoparticle having a hydroxyl group on the surface to introduce an amino group, while a thiol group introduction reagent (for example, N-succimidyl S acetylthioacetic acid) is reacted with streptavidin to introduce a thiol group, and finally, a PEG (polyethylene glycol)-based linker molecule having a maleimide group reactive with both the amino group and the thiol group at both ends is reacted to link luminescent nanoparticle and streptavidin.

In addition, for example, when a resin (acrylic-based resin) is synthesized using glycidyl methacrylate as a raw material monomer, an epoxy-group derived from the monomer appears on a surface of the luminescent nanoparticle. By adding ammonia water to the luminescent nanoparticle, the epoxy-group can be converted to an amino-group, and the desired target directing ligand or the like can be linked to the amino-group.

### EXAMPLE

Hereinafter, the present invention will be described in detail with reference to Example, but the present invention is not limited thereto. In Example, “part” or “%” is used, but unless otherwise specified, “parts by mass” or “% by mass” is used.

### <<Luminescent Compounds>>

### <Luminescent Compounds (Excited Dye) Used in Comparative Example>

In Comparative Example, a luminescent compound represented by the following structural formula was used in place of the first luminescent compound. Hereinafter, the luminescent compound is referred to as Cf. In the luminescent compound (Cf), the maximum absorption wavelength λ_{ab1} is 338 nm and the maximum luminescence wavelength λₑₘ₁ is 345 nm. The luminescent compound (Cf) is a compound that does not transfer exciting energy to the second luminescent compound because the luminescent spectrum does not overlap with the absorbance spectrum of the second A-1 shown below. In the resulting nanoparticles, no luminescence from the second luminescent compound is observed, which confirms that the excited energy does not migrate.

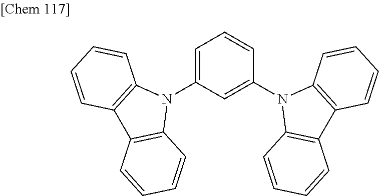 [SMILES: c1cc(-n2c3ccccc3c3ccccc32)cc(-n2c3ccccc3c3ccccc32)c1]

### <First Luminescent Compound Used in Example>

In Example, the following compound (C-167) was used as the first luminescent compound. In the compound (C-167), the maximum absorption wavelength λ_{ab1} is 570 nm and the maximum luminescence wavelength λₑₘ₁ is 608 nm.

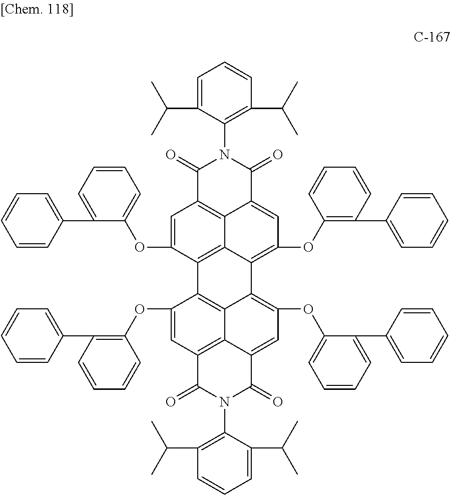 [SMILES: CC(C)c1cccc(C(C)C)c1N1C(=O)c2cc(Oc3ccccc3-c3ccccc3)c3c4c(Oc5ccccc5-c5ccccc5)cc5c6c(cc(Oc7ccccc7-c7ccccc7)c(c7c(Oc8ccccc8-c8ccccc8)cc(c2c37)C1=O)c64)C(=O)N(c1c(C(C)C)cccc1C(C)C)C5=O]

### <Second Luminescent Compound Used in Example>

In Example, the following compounds were used as the second luminescent compound: Hereinafter, the luminescent compound is referred to as A-1. In the luminescent compound (A-1), the maximum absorption wavelength λ_{ab2} is 655 nm and the maximum luminescence wavelength λₑₘ₂ is 681 nm.

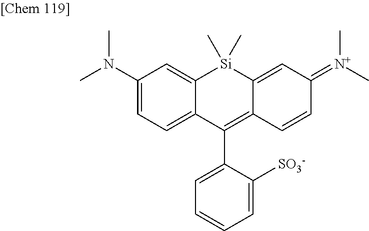

### <<Preparation of Luminescent Nanoparticle>>

### [Example 1 and Comparative Examples] Preparation of Luminescent Nanoparticle Using Oil Soluble Dyes

### <Preparation of Luminescent Nanoparticle Nos. (1-13) to (1-19)>

### [Example 1-13] Preparation of Luminescent Nanoparticle Using Oil Soluble Dyes

The first luminescent compound (C-167) and the second luminescent compound (A-1) were dissolved in 25 mg/ml and 2.5 mg/ml in dichloromethane of 0.168 mL, respectively, and Emulgen 430 (5% by mass aqueous solution) of 0.312 mL and DBS-Na (1.62 weight % aqueous solution) of 0.381 mL were added to it.

Ultrasonic waves were applied using an ultrasonic homogenizer UH150 for 5 minutes while being cooled in ice-water, and then dichloromethane was removed by vacuum-pumping while stirring at 300 rpm. After the solution was heated and stirred on a hot stirrer at 82° C. and for 15 minutes, a melamine resin (Nicarac MX-035 (manufactured by Nippon Carbide Industry Co., Ltd., solid content 50% by mass aqueous solution) of 0.07 mL was added with stirring, and after stirring for 2 minutes, a aqueous solution of 0.1 mL, in which DBS (dodecylbenzenesulfonic acid) of 1% by mass and TsOH (p-toluenesulfonic acid) of 0.33% by mass were mixed, were added, and further heated and stirred for 90 minutes. After completion of the heating and stirring, heating by an autoclave at 121° C. and for 40 minutes was carried out.

The resulting dispersions were centrifuged at 18500 G for 10 minutes to remove the supernatant, and then ultrapure water was added and redispersed with a homogenizer. The treatment by removal of the supernatant after centrifugation and redispersion into ultrapure water was repeated five times. In addition, an organic solvent was used instead of ultrapure water, and a cleaning operation was performed until the color of the supernatant liquid was not visible, and luminescent nanoparticle No. (1-13) was obtained.

### Comparative Examples 1-1 to 1-5 and Examples 1-14 to 1-19

After the first luminescent compound and the second luminescent compound described in Tables I were dissolved in dichloromethane of 0.168 mL to a predetermined level, the luminescent nanoparticles Nos. (1-1) to (1-5), (1-14) to (1-19) were obtained according to the same conditions as in Examples 1-13.

Table I shows a difference λ_{ab2}−λₑₘ₁ between the maximum luminescence wavelength of the first luminescent compound and the maximum absorption wavelength of the second luminescent compound, and the maximum luminescence wavelength λₑₘ₂ of the second luminescent compound.

### <<Calculation of Content of Luminescent Compound in Luminescent Nanoparticle>>

Each of the luminescent nanoparticles Nos. (1-1) to (1-19) prepared above was dispersed in ultrapure water so as to have concentration of 0.0189 mg/mL, and absorption spectrums of the nanoparticles were measured with an absorption photometer spectrophotometer (U-3300, Hitachi High-Tech Science Co., Ltd.) at room temperature. A content of each of the luminescent compounds contained in each of the nanoparticles was calculated from the maximum absorption wavelength corresponding to each of the luminescent compounds and a molar absorption coefficient of each of the luminescent compounds.

### <<Evaluation of Luminescence Intensities of Luminescent Nanoparticles>>

Each of the luminescent nanoparticles was dispersed in ultrapure water so as to have concentration of 0.0189 mg/mL, and a luminescence spectrum of each dispersion was measured with a fluorometer (Hitachi High-Technologies, F-7000) by exciting, at the maximum absorption wavelength of the first luminescent compound in each of the nanoparticles, the first luminescent compound at room temperature. However, a luminescence spectrum of Comparative Example 1-2 was measured by exciting, at the maximum absorption wavelength of its second luminescent compound, its second luminescent compound.

In the luminescence spectrum of Comparative Example 1-1 of the luminescent nanoparticles prepared above, its maximum luminescence wavelength was present in a luminescent peak originating from its first luminescent compound, but a luminescent peak originating from its second luminescent compound was not confirmed. Meanwhile, in the luminescence spectrum of each of Comparative Examples 1-2 to 1-5 and Examples 1-13 to 1-19, each maximum luminescence wavelength was present in a luminescent peak originating from each second luminescent compound.

The maximum luminescence wavelength and a relative value between the luminescence intensities obtained from the luminescence spectrum of each of Comparative Examples 1-2 to 1-5 and Examples 1-13 to 1-19 are shown in Table I. The luminescence intensity (relative value) of the respective particles is obtained by setting the measured value of the luminescent nanoparticle of Comparative Example 1-2 to 1.

It can be seen that when a content of the first luminescent compound is 4 to 90% by mass, the relative luminescence intensity is higher than that of each of Comparative Examples, and when a content is 30% by mass, the luminescence intensity is the maximum.

TABLE 1
Table I

| | | ABBREVIATIONS OF COMPOUND | | CONTENT [MASS %] | |
|---|---|---|---|---|---|
| | | FIRST | SECOND | FIRST | SECOND |
| | | LUMINESCENT | LUMINESCENT | LUMINESCENT | LUMINESCENT |
| | ARTICLES | COMPOUND | COMPOUND | COMPOUND | COMPOUND |
| COMPARATIVE | 1-1 | Cf | A-1 | >2.00 | 0.47 |
| EXAMPLE 1-1 | | | | | |
| COMPARATIVE | 1-2 | — | A-1 | 0 | 0.48 |
| EXAMPLE 1-2 | | | | | |
| COMPARATIVE | 1-3 | C-167 | A-1 | 1 | 0.14 |
| EXAMPLE 1-3 | | | | | |
| COMPARATIVE | 1-4 | C-167 | A-1 | 90.3 | 0.14 |
| EXAMPLE 1-4 | | | | | |
| COMPARATIVE | 1-5 | C-167 | A-1 | 1.9 | 0.24 |
| EXAMPLE 1-5 | | | | | |
| EXAMPLE 1-13 | 1-13 | C-167 | A-1 | 4.86 | 0.2 |
| EXAMPLE 1-14 | 1-14 | C-167 | A-1 | 18.5 | 0.39 |
| EXAMPLE 1-15 | 1-15 | C-167 | | 16.6 | 0.14 |
| EXAMPLE 1-16 | 1-16 | C-167 | A-1 | 30.1 | 0.13 |
| EXAMPLE 1-17 | 1-17 | C-167 | A-1 | 44.7 | 0.15 |
| EXAMPLE 1-18 | 1-18 | C-167 | A-1 | 59.4 | 0.11 |
| EXAMPLE 1-19 | 1-19 | C-167 | A-1 | 76.3 | 0.12 |

| | | | MAXIMUM | LUMINESCENCE |
|---|---|---|---|---|
| | | | LUMINESCENCE | INTENSITY |
| | MOLAR RATIOS | λ_{ab2}-λₑₘ₁ | WAVELENGTH | (RELATIVE |
| | FIRST: SECOND | [nm | [nm] | VALUE) |
| COMPARATIVE | — | 310 | — | — |
| EXAMPLE 1-1 | | | | |
| COMPARATIVE | — | — | 685 | 1 |
| EXAMPLE 1-2 | | | | |
| COMPARATIVE | 1.6:1 | 47 | 685 | 1.9 |
| EXAMPLE 1-3 | | | | |
| COMPARATIVE | 209:1 | 47 | 685 | 2.5 |
| EXAMPLE 1-4 | | | | |
| COMPARATIVE | 8:1 | 47 | 685 | 2.1 |
| EXAMPLE 1-6 | | | | |
| EXAMPLE 1-13 | 8:1 | 47 | 685 | 3.9 |
| EXAMPLE 1-14 | 14:1 | 47 | 685 | 6.8 |
| EXAMPLE 1-15 | 39:1 | 47 | 685 | 8.7 |
| EXAMPLE 1-16 | 71:1 | 47 | 685 | 10.1 |
| EXAMPLE 1-17 | 105:1 | | 685 | 9.7 |
| EXAMPLE 1-18 | 139:1 | 47 | 685 | 8.9 |
| EXAMPLE 1-19 | 174:1 | 47 | 685 | 7.4 |

### <<Preparation of Luminescent Labeling Material for Pathological Diagnosis Use>>

### [Example 2] Luminescent Labeling Material for Pathological Diagnosis Use Formed by Luminescent Nanoparticle No. (1-16)

<Preparation of Luminescent Nanoparticle Having Surface Thereof Modified with PEG Chain Having Maleimide Group at End Thereof>

The luminescent nanoparticle No. (1-16) of 0.1 mg, which is a melamine particle containing a luminescent compound, was dispersed in an ethanolic 1.5 mL, and aminopropyltrimethoxysilane “LS-3150” (manufactured by Shin-Etsu Chemical Co., Ltd.) of 2 μL was added thereto, and the mixture was subjected to a surface-amination treatment by stirring for 8 hours at room temperature to induce reaction.

The concentration of the surface-aminated luminescent nanoparticle was adjusted to 3 nM using PBS (phosphate buffered physiological saline) containing EDTA (ethylenediaminetetraacetic acid) of 2 mM, and the linker reagent “SM(PEG)12” (Thermo Scientific, cat. No. 22112) was added and mixed to this solution so as to give a final concentration of 10 mM, and stirred for 1 hour at room temperature to induce reaction.

The reaction liquid was centrifuged at 10,000 G for 20 minutes, the supernatant was removed, and then PBS containing EDTA of 2 mM was added thereto to disperse the precipitate, and the precipitate was centrifuged again under the same conditions. Three times of wash in the same manner were performed to obtain the luminescent nanoparticle having a surface thereof modified with a PEG chain having a maleimide group at an end thereof.

<Preparation of Streptavidin Introduced with Thiol Group>

First, an aqueous solution of N-succinimidyl-S-acetylthioacetate (manufactured by Wako Pure Chemical Industries, Ltd.) of 70 μL which was adjusted to 64 mg/mL was added to an aqueous solution of streptavidin (manufactured by Wako Pure Chemical Industries, Ltd.) of 40 μL which was adjusted to 1 mg/mL, and caused to react for 1 hour at room temperature to introduce a protected thiol group (—NH—CO—CH₂—S—CO—CH₃) to the amino group of streptavidin.

Subsequently, hydroxylamine treatment is carried out, which causes to generate a free thiol group (—SH) from the protected thiol group, and thereby the process of introducing a thiol group (—SH) into streptavidin is completed. This solution was desalted through a gel-filtration column (Zaba Spin Desalting Columns: Funakoshi) to obtain streptavidin to which a thiol group was introduced.

<Preparation of Luminescent Nanoparticle Modified with Streptavidin>

The prepared luminescent nanoparticle having a surface thereof modified with a PEG chain having a maleimide group at an end thereof and the prepared streptavidin to which a thiol group was introduced were mixed in PBS containing EDTA of 2 mM to cause reaction for 1 hour, which bound streptavidin to the luminescent nanoparticle via the PEG chain. Mercaptoethanol of 10 mM was added to the reaction solution to stop the reaction. The obtained solutions were concentrated by a centrifugal filter, and then the unreacted substances were removed using a gel filtration column for purification to obtain a luminescent labeling material for pathological diagnosis use (luminescent nanoparticle modified with streptavidin).

### [Comparative Example 2] Luminescent Labeling Material for Pathological Diagnosis Use Formed by Luminescent Nanoparticle No. (1-2)

The luminescent labeling material for pathological diagnosis use (luminescent nanoparticle modified with streptavidin) was obtained by the same experimental procedure as in Example 2.

### Example 3: Evaluation for Luminescent Labeling Material for Pathological Diagnosis Use

### <Tissue Dyeing Step>

### [Immunohistochemical Dyeing]

Immunodyeing of human breast tissue was performed using dyes for tissue dyeing including the luminescent labeling materials for pathological diagnosis use formed by the luminescent nanoparticles prepared in Example 2 and Comparative Example 2. Here, the dyes for tissue dyeing were prepared using a PBS buffer containing BSA of 1%. Tissue array slides (Cosmo Bio, part number CB-A712) were used for the dyed sections.

FISH scores per spot in the dyed sections were calculated in advance using a PathVysion HER-2 DNA Probe Kit (manufactured by Abbott Corporation). The calculation of the FISH scores was performed according to the procedures described in the document attached to the HER-2 DNA Probe Kit manufactured by Abbott Japan, PathVysion®; HER-2 DNA Probe Kit.

The tissue array slides were subjected to deparaffinization, then displacement washing, and autoclaved treatment for 15 minutes in citrate buffer (pH 6.0) of 10 mM, so as to perform antigen activating treatment. The tissue array slides after antigen activating treatment were washed with PBS buffer, and then was caused to react with anti HER2 rabbit monoclonal antibody (4B5) diluted to be 0.05 nM with PBS buffer containing BSA of 1%, for 2 hours. After washing with PBS, it was caused to react with biotin-labeled anti-rabbit antibody diluted with PBS buffer containing BSA of 1%, for 30 minutes. Furthermore, it was caused to react with the above-described dyeing agents for tissue dyeing, that is, the prepared luminescent labeling materials for pathological diagnosis use (luminescent nanoparticles including streptavidin), for 2 hours and then washed, so as to obtain immunohistochemically dyed sections. The obtained immunohistochemically dyed sections were immersed in neutral paraformaldehyde aqueous buffer of 4%, for 10 minutes, so as to perform the immobilization treatment.

### [Morphological Dyeing]

HE dyeing was performed on the immunohistochemically dyed sections which had been subjected to the immobilization treatment, dehydration treatment was performed on the dyed sections by immersion in ethanol, and permeating treatment was performed on the dehydrated sections by further immersion in xylene and drying in air, and thereby the double dyed sections were obtained.

### [Encapsulation]

Entellan New (manufactured by Merck Co., Ltd.), which is a xylene-based encapsulant, was added dropwise to the double dyed sections which had been subjected to the morphological dyeing, and covered with a cover glass to be encapsulated.

### <Evaluation of Tissue Samples>

Shapes of cells (position of cell membranes) were identified by image processing using dyed images for morphological observation and superimposed with the immunohistochemically dyed images, and then microscopic observation was carried out via the luminescent labeling materials for pathological diagnosis use (luminescent nanoparticles modified with streptavidin which were formed by the luminescent nanoparticles) with which were labeled HER2 protein appearing on the cell membranes and which were subjected to irradiation of excitation light. It was possible to confirm a bright spot due to the nanoparticle prepared in Example 2 while it was difficult to confirm a bright spot due to the nanoparticle prepared in Comparative Example 2 in which the Stokes shift was less than 50 nm because of the effect of cellular autofluorescence. This result shows that the inventive luminescent nanoparticle can be used as a luminescent labeling material for pathological diagnosis use.

### INDUSTRIAL APPLICABILITY

According to the present invention, it is possible to provide a luminescent nanoparticle and a luminescent labeling material for pathological diagnosis use that realize a high-brightness particulate technique for bioimaging and enable high-sensitivity imaging.

1. A luminescent nanoparticle comprising a first luminescent compound and a second luminescent compound, wherein
the first luminescent compound is excited by light irradiation and has a function of transferring energy due to the excitation to the second luminescent compound,
the second luminescent compound has a function of receiving the energy due to the excitation to emit light, and
a content of the first luminescent compound to a total amount of the luminescent nanoparticle is in the range of 4 to 90% by mass.

2. The luminescent nanoparticle according to claim 1, wherein the first luminescent compound has a structure represented by the following general formula (1), general formula (2), or general formula (3): 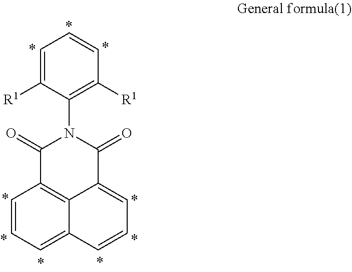
in the general formula (1), each of the plurality of R¹ independently represents a hydrogen atom or a substituent, and at least one of them represents a monovalent organic group having 3 to 30 carbon atoms; the benzene ring or the naphthalene ring may further have a substituent, and * represents a position of the substituent which may be included in the benzene ring or the naphthalene ring, 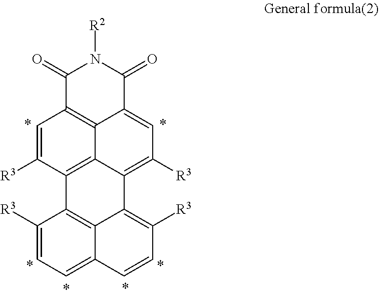
in the general formula (2), R² represents a substituted or unsubstituted alkyl group, an aryl group or a heteroaryl group; each of the plurality of R³ independently represents a hydrogen-atom or a group having a structure represented by the following general formula (F1), and at least one of them represents a group having a structure represented by the following general formula (F1); the naphthalene ring may further have a substituent, and * represents a position of the substituent that may be included in the naphthalene ring, 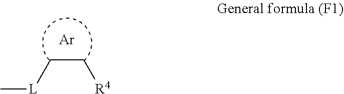
in the general formula (F1), Ar represents an aryl ring or a heteroaryl ring; R⁴ represents a substituent; when two or more groups represented by the general formula (F1) are included, the two R⁴ may be linked to each other; L represents a single bond, an oxygen-atom, a sulfur-atom or —NR′—; R′ represents a hydrogen atom, an alkyl group, an aryl group or a heteroaryl group, 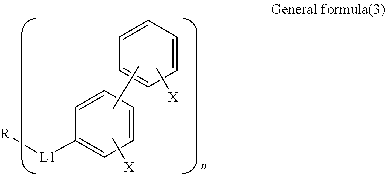
in the general formula (3), R represents a luminescent compound skeleton; each X independently represents an ionic substituent; L1 represents a single bond, an oxygen-atom, a sulfur-atom, a selenium-atom or a NH group; and n represents an integer of 1 or more.

3. The luminescent nanoparticle according to claim 1, wherein the first luminescent compound comprises a compound having a structure represented by the following general formula (1c), general formula (1d), or general formula (1e): 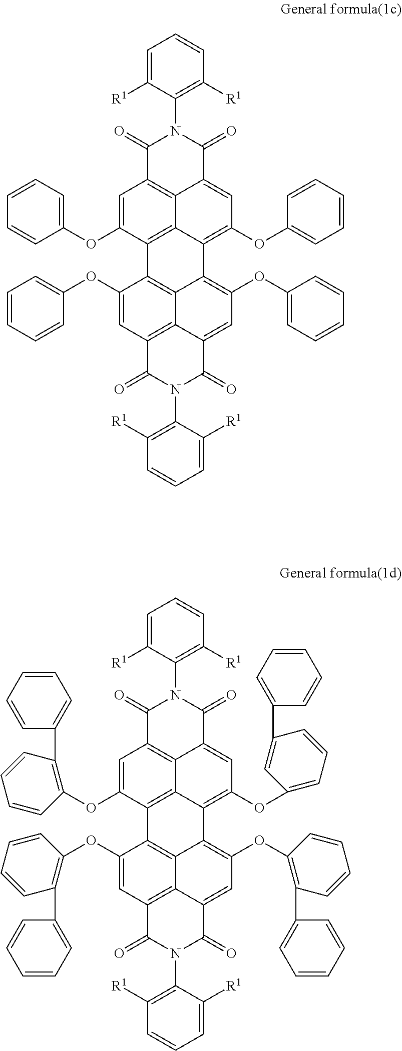 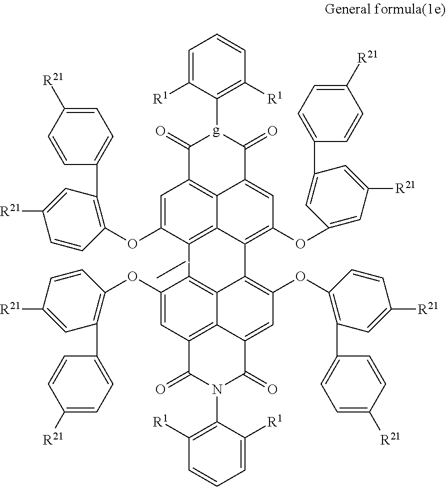
in general formulae (1c) to (1e), the plurality of R each independently represents a hydrogen atom or a substituent, and at least one of them represents a monovalent organic group having 3 to 30 carbon atoms; in the general formula (1e), R²¹ independently represents a hydrogen-atom or an ionic substituent, and at least one of them represents an ionic substituent.

4. The luminescent nanoparticle according to claim 1, wherein a molar ratio of the second luminescent compound to the first luminescent compound is in the range of 1:2 to 1:200.

5. The luminescent nanoparticle according to claim 1, wherein the second luminescent compound is a xanthene dye.

6. The luminescent nanoparticle according to claim 1, further comprising a binder.

7. The luminescent nanoparticle according to claim 1, having a hydrophilic group on a surface thereof.

8. A luminescent labeling material for pathological diagnosis use in which the luminescent nanoparticle according to claim 1 is used.
